(19) United States
(12) Patent Application Publication
(10) Pub. No.: US 2024/0226302 A1
(43) Pub. Date: Jul. 11, 2024

(54) BIODEGRADABLE LIPIDS AND FORMULATIONS FOR INTRAMUSCULAR, IN VITRO, AND EX VIVO TRANSFECTION OF MRNA

(71) Applicants: Massachusetts Institute of Technology, Cambridge, MA (US); Children's Medical Center Corporation, Boston, MA (US)

(72) Inventors: Daniel Griffith Anderson, Framingham, MA (US); Amab Rudra, Brookline, MA (US); Akash Gupta, Arlington, MA (US); Kaelan Reed, Cambridge, MA (US)

(73) Assignees: Massachusetts Institute of Technology, Cambridge, MA (US); Children's Medical Center Corporation, Boston, MA (US)

(21) Appl. No.: 18/396,181

(22) Filed: Dec. 26, 2023

Related U.S. Application Data
(60) Provisional application No. 63/435,530, filed on Dec. 27, 2022.

(51) Int. Cl. A61K 47/22; A61K 31/7105; C07D 257/02

(52) U.S. Cl. CPC A61K 47/22; A61K 31/7105; C07D 257/02

(57) ABSTRACT

Provided herein are compounds, such as compounds of Formula (I), and pharmaceutically acceptable salts thereof, and compositions, methods, uses, and kits thereof. The compounds provided herein are lipids useful for delivery of agents, including polynucleotides such as mRNA, for the treatment and/or prevention of various diseases and conditions (e.g., genetic diseases, proliferative diseases, hematological diseases, neurological diseases, liver diseases, spleen diseases, lung diseases, painful conditions, psychiatric disorders, musculoskeletal diseases, metabolic disorders, inflammatory diseases, and autoimmune diseases). Also provided herein are methods of synthesis of compounds of Formulae (I) and (II).

### CROSS REFERENCE TO RELATED APPLICATIONS

This application claims the benefit of and priority under 35 U.S.C. § 119(e) to U.S. Provisional Application No. 63/435,530, filed Dec. 27, 2022, titled BIODEGRADABLE LIPIDS AND FORMULATIONS FOR INTRAMUSCULAR, IN VITRO, AND EX VIVO TRANSFECTION OF MRNA, the contents of which are incorporated herewith by reference in their entirety.

### BACKGROUND

mRNA therapeutics have significant potential to treat various diseases through protein replacement, immunomodulation, and gene editing. Non-viral nanoparticles are promising mRNA delivery vehicles to target cells in vivo. For example, mRNA-based vaccines against SARS-CoV-2 has highlighted the utility of lipid nanoparticle formulations for intramuscular administration. New compounds capable of forming delivery vehicles may be useful for mRNA-based vaccines and chimeric antigen receptor T (CAR-T) cell therapy.

### SUMMARY OF THE INVENTION

Provided herein are biodegradable ionizable lipids that, when incorporated in optimized formulations containing mRNA, demonstrate efficient in vivo transfection. The lipids provided herein are also suitable for in vitro and ex vivo transfection of mRNA to hard-to-transfect cells.

In one aspect, provided herein is a compound of Formula (I):

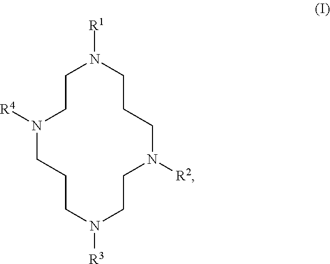

or a pharmaceutically acceptable salt thereof, wherein R¹, R², R³, and R⁴ are as defined herein.

In another aspect, provided herein is a composition comprising a compound provided herein, or a pharmaceutically acceptable salt thereof, and a pharmaceutically acceptable excipient.

In another aspect, provided herein is a method of delivering an agent to a subject or a cell, comprising administering to the subject or contacting the cell with a composition comprising an agent and a compound provided herein, or a pharmaceutically acceptable salt thereof.

In another aspect, provided herein is a method of treating and/or preventing a disease, disorder, or condition in a subject, comprising administering to the subject a composition comprising an agent and a compound provided herein, or a pharmaceutically acceptable salt thereof.

In another aspect, provided herein is a method of preparing a compound of Formula (II):

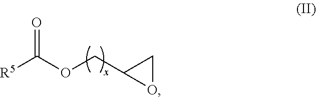

the method comprising reacting a compound of Formula (III):

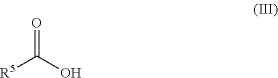

or a salt thereof, with a carbodiimide coupling agent, or a salt thereof, and a compound of Formula (IV):

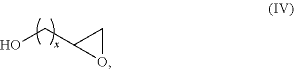

or a salt thereof, wherein x and R⁵ are as defined herein.

In another aspect, provided herein is a method of preparing a compound of Formula (I):

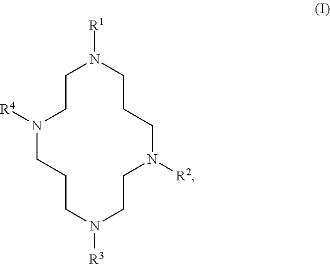

or a salt thereof, wherein R¹, R², R³, and R⁴ are as defined herein.

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which constitute a part of this specification, illustrate several embodiments of the invention and together with the description, provide non-limiting examples of the invention.

FIG. 1A shows structures of the fatty acid epoxy esters and amine used for ionizable lipids. FIG. 1B shows the synthetic route for the preparation of G1-G5. FIG. 1C shows the synthetic route to G6. FIG. 1D shows the synthetic route for G8-G10 (n=1, 3, 5). FIG. 1E shows the synthetic route to SG1-SG3 (n=1, 3, 5).

FIG. 2 shows a heatmap of in vitro performance of the ionizable lipids relative to performance of MD-1.

FIG. 3 shows relative luminescence of ionizable lipids AMG104 and MD1.

FIG. 4 shows additional fatty acid epoxy esters and amine headgroups AM12 and AM13.

FIG. 5A shows luciferase expression after intramuscular injection of Fluc mRNA at 0.25 mg/kg dose at 6 h timepoint. FIG. 5B shows luciferase expression after intramuscular injection of Fluc mRNA at 0.25 mg/kg dose at 6 h, 24 h, and 48 h time points.

FIG. 6 shows screening results for ionizable lipid AMG104 using DOPE (left, 1-14) and DSPC (right, 1-14) as the helper lipid (error bars=S.D., n=3 wells).

FIG. 7 shows in vitro full factorial screening results for AMG104 (error bars=S.D., n=3 wells).

FIG. 8 shows structures of ionizable lipids.

FIGS. 9A-9B shows structures of fatty acid epoxy esters used for ionizable lipids. FIG. 9A shows structures of fatty acid epoxy esters in which the position of the Z-alkene was varied. FIG. 9B shows structures of fatty acid epoxy esters and an alkene-based tail.

FIG. 10 shows an orientation of ionizable lipid AMG1041-OHD.

FIG. 11 shows relative luminescence of ionizable lipids in C2C12 myoblast cells.

FIG. 12 shows luciferase expression after intramuscular administration of lipids with FLuc mRNA (0.1 μg).

FIG. 13 shows luciferase expression after intramuscular administration of chirally pure lipids with FLuc mRNA.

FIG. 14 shows antibody titers after intramuscular injection with Ova mRNA and AMG1041.

FIGS. 15A-15B show lipid clearance over time after intramuscular injection to BALB/C mice. FIG. 15A shows clearance of AMG1041. FIG. 15B shows clearance of SM-102.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In a formula, the bond [symbol] is a single bond, the dashed line --- is a single bond or absent and the bond [symbol] or [symbol] is a single or double bond.

[symbol]

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr. e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

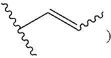

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 xT electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{aa})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{cc})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNRbbS(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R)₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{f} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₀₋₆ alkyl, —OR^{aa}, —SR^{a}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R—, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X— are as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{b}b)₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂—CO₂R^{aa}, —SO₂R^{aa}, —C(═NR^{bb})R—, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₀₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₀₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₀₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₀₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R′, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The disclosure is not intended to be limited in any manner by the above exemplary listing of substituents. Additional terms may be defined in other sections of this disclosure.

### Other Definitions

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N+(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “isotopes” refers to variants of a particular chemical element such that, while all isotopes of a given element share the same number of protons in each atom of the element, those isotopes differ in the number of neutrons.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

As used herein, “lipophilic” refers to the ability of a group to dissolve in fats, oils, lipids, and lipophilic non-polar solvents such as hexane or toluene. In general, a lipophilic group refers to an unsubstituted n-alkyl or unsubstituted n-alkenyl group having 6 to 50 carbon atoms, e.g., 6 to 40, 6 to 30, 6 to 20, 8 to 20, 8 to 19, 8 to 18, 8 to 17, 8 to 16, or 8 to 15 carbon atoms. The terms “phosphorylethanolamine” and “phosphoethanolamine” are used interchangeably.

The term “sterol” refers to a subgroup of steroids also known as steroid alcohols, i.e., a steroid containing at least one hydroxyl group. Sterols are usually divided into two classes: (1) plant sterols also known as “phytosterols,” and (2) animal sterols also known as “zoosterols.” The term “sterol” includes, but is not limited to, cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers.

As used here, the term “PEG-lipid” refers to a PEGylated lipid.

An “amino acid” refers to natural and unnatural D/L alpha-amino acids, as well as natural and unnatural beta- and gamma-amino acids. A “peptide” refers to two amino acids joined by a peptide bond. A “polypeptide” refers to three or more amino acids joined by peptide bonds. An “amino acid side chain” refers to the group(s) pended to the alpha carbon (if an alpha amino acid), alpha and beta carbon (if a beta amino acid), or the alpha, beta, and gamma carbon (if a gamma amino acid). Exemplary amino acid side chains are depicted herein.

A “protein,” “peptide,” or “polypeptide” comprises a polymer of amino acid residues linked together by peptide bonds. The term, as used herein, refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “apolipoprotein” refers to a protein that binds a lipid (e.g., triacylglycerol or cholesterol) to form a lipoprotein. Apolipoproteins also serve as enzyme cofactors, receptor ligands, and lipid transfer carriers that regulate the metabolism of lipoproteins and their uptake in tissues. Major types of apolipoproteins include integral and non-integral apolipoproteins. Exemplary apolipoproteins include apoA (e.g., apoA-I, apoA-II, apoA-IV, and apoA-V); apoB (e.g., apoB48 and apoB 100); apoC (e.g., apoC-I, apoC-II, apoC-III, and apoC-IV); apoD; apoE; apoH; and apoJ.

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. The oligonucleotide may comprise one or more locked nucleic acid (LNA) moieties. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), a provirus, a lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), a polyinosinic acid, a ribozyme, a flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Such promoters include, but are not limited to: the SV40 early promoter region (Bernoist et al., Nature, 290, 304-310, (1981); Yamamoto et al., Cell, 22, 787-797, (1980); Wagner et al., Proc. Natl. Acad. Sci. U.S.A. 78, 1441-1445, (1981); Brinster et al., Nature 296, 39-42, (1982)). Any type of plasmid, cosmid, yeast artificial chromosome or viral vector can be used to prepare the recombinant DNA construct that can be introduced directly into the tissue site. Alternatively, viral vectors can be used which selectively infect the desired tissue, in which case administration may be accomplished by another route (e.g., systemically).

The polynucleotides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “recombinant nucleic acid molecule” is a nucleic acid molecule that has undergone a molecular biological manipulation, i.e., non-naturally occurring nucleic acid molecule or genetically engineered nucleic acid molecule. Furthermore, the term “recombinant DNA molecule” refers to a nucleic acid sequence which is not naturally occurring, or can be made by the artificial combination of two otherwise separated segments of nucleic acid sequence, i.e., by ligating together pieces of DNA that are not normally continuous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature. Restriction enzyme recognition sites are often the target of such artificial manipulations, but other site specific targets, e.g., promoters, DNA replication sites, regulation sequences, control sequences, open reading frames, or other useful features may be incorporated by design. Examples of recombinant nucleic acid molecule include recombinant vectors, such as cloning or expression vectors which contain DNA sequences encoding Ror family proteins or immunoglobulin proteins which are in a 5′ to 3′ (sense) orientation or in a 3′ to 5′ (antisense) orientation.

The term “pDNA,” “plasmid DNA,” or “plasmid” refers to a small DNA molecule that is physically separate from, and can replicate independently of, chromosomal DNA within a cell. Plasmids can be found in all three major domains: Archaea, Bacteria, and Eukarya. In nature, plasmids carry genes that may benefit survival of the subject (e.g., antibiotic resistance) and can frequently be transmitted from one bacterium to another (even of another species) via horizontal gene transfer. Artificial plasmids are widely used as vectors in molecular cloning, serving to drive the replication of recombinant DNA sequences within host subjects. Plasmid sizes may vary from 1 to over 1,000 kbp. Plasmids are considered replicons, capable of replicating autonomously within a suitable host.

“RNA transcript” refers to the product resulting from RNA polymerase-catalyzed transcription of a DNA sequence. When the RNA transcript is a complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “gene silencing” refers to an epigenetic process of gene regulation where a gene is “switched off” by a mechanism other than genetic modification. That is, a gene which would be expressed (i.e., “turned on”) under normal circumstances is switched off by machinery in the cell. Gene silencing occurs when RNA is unable to make a protein during translation. Genes are regulated at either the transcriptional or post-transcriptional level. Transcriptional gene silencing is the result of histone modifications, creating an environment of heterochromatin around a gene that makes it inaccessible to transcriptional machinery (e.g., RNA polymerase and transcription factors). Post-transcriptional gene silencing is the result of mRNA of a particular gene being destroyed or blocked. The destruction of the mRNA prevents translation and thus the formation of a gene product (e.g., a protein). A common mechanism of post-transcriptional gene silencing is RNAi.

As used herein, a “chimeric receptor” or “chimeric antigen receptor” refers to a non-naturally occurring molecule that can be expressed on the surface of a host cell and comprises binding domain that provides specificity of the chimeric receptor (e.g., an antigen-binding fragment that binds to an epitope of a cell surface protein). In general, chimeric receptors comprise at least two domains that are derived from different molecules. In addition to the epitope-binding fragment described herein, the chimeric receptor may further comprise one or more of the following: a hinge domain, a transmembrane domain, a co-stimulatory domain, a cytoplasmic signaling domain, and combinations thereof. In some embodiments, the chimeric receptor comprises from N terminus to C terminus, an antigen-binding fragment that binds to a cell surface protein, a hinge domain, a transmembrane domain, and a cytoplasmic signaling domain. In some embodiments, the chimeric receptor further comprises at least one co-stimulatory domain. See, e.g., Marin-Acevedo et al. J. Hematol. Oncol. (2018) 11: 8.

In some embodiments, the chimeric receptors described herein comprise one or more hinge domain(s). In some embodiments, the hinge domain may be located between the antigen-binding fragment and a transmembrane domain. A hinge domain is an amino acid segment that is generally found between two domains of a protein and may allow for flexibility of the protein and movement of one or both of the domains relative to one another. Any amino acid sequence that provides such flexibility and movement of the antigen-binding fragment relative to another domain of the chimeric receptor can be used.

In some embodiments, the chimeric receptors described herein may comprise one or more transmembrane domain(s). The transmembrane domain for use in the chimeric receptors can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, the transmembrane domain may be a synthetic, non-naturally occurring protein segment, e.g., a hydrophobic protein segment that is thermodynamically stable in a cell membrane.

In some embodiments, the chimeric receptors described herein comprise one or more costimulatory signaling domains. The term “co-stimulatory signaling domain,” as used herein, refers to at least a portion of a protein that mediates signal transduction within a cell to induce an immune response, such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles (e.g., lipid nanoparticles), macroscopic particles, pellets, crystals, aggregates, composites, pulverized, milled or otherwise disrupted matrices, and cross-linked protein or polysaccharide particles, each of which have an average characteristic dimension of about less than about 1 mm and at least 1 nm, where the characteristic dimension, or “critical dimension,” of the particle is the smallest cross-sectional dimension of the particle. A particle may be composed of a single substance or multiple substances. In certain embodiments, the particle is not a viral particle. In other embodiments, the particle is not a liposome. In certain embodiments, the particle is not a micelle. In certain embodiments, the particle is substantially solid throughout. In certain embodiments, the particle is a nanoparticle. In certain embodiments, the particle is a microparticle.

The terms “chimeric antigen receptor” or “CAR” refer to an engineered receptor or recombinant polypeptide construct comprising an extracellular antigen binding domain (e.g., derived from a monoclonal antibody), a transmembrane domain, and an intracellular effector domain (e.g., a functional signaling domain derived from a stimulatory molecule).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher-than-normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound or agent described herein refers to an amount sufficient to elicit the desired biological response. An effective amount of a compound or agent described herein may vary depending on such factors as the desired biological endpoint, severity of side effects, disease, or disorder, the identity, pharmacokinetics, and pharmacodynamics of the particular compound or agent, the condition being treated, the mode, route, and desired or required frequency of administration, the species, age and health or general condition of the subject. In certain embodiments, an effective amount is a therapeutically effective amount. In certain embodiments, an effective amount is a prophylactically effective amount. In certain embodiments, an effective amount is the amount of a compound or agent described herein in a single dose. In certain embodiments, an effective amount is the combined amounts of a compound or agent described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound or agent described herein is an amount sufficient to provide a therapeutic benefit in the treatment of a condition or to delay or minimize one or more symptoms associated with the condition. A therapeutically effective amount of a compound or agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for delivering an agent to a subject or a cell. In certain embodiments, a therapeutically effective amount is an amount sufficient for delivering a polynucleotide to a subject or a cell. In certain embodiments, a therapeutically effective amount is an amount sufficient for delivering mRNA to a subject or a cell. In certain embodiments, a therapeutically effective amount is an amount sufficient for treating a disease, disorder, or condition. In certain embodiments, a therapeutically effective amount is an amount sufficient for delivering an agent to a subject or a cell and treating a disease, disorder, or condition. In certain embodiments, a therapeutically effective amount is an amount sufficient for delivering a polynucleotide to a subject or a cell and treating a disease, disorder, or condition. In certain embodiments, a therapeutically effective amount is an amount sufficient for delivering mRNA to a subject or a cell and treating a disease, disorder, or condition. In certain embodiments, a therapeutically effective amount is an amount effective for chimeric antigen receptor T cell therapy.

A “prophylactically effective amount” of a compound or agent described herein is an amount sufficient to prevent a condition, or one or more symptoms associated with the condition or prevent its recurrence. A prophylactically effective amount of a compound or agent means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for delivering an agent to a subject or a cell. In certain embodiments, a prophylactically effective amount is an amount sufficient for delivering a polynucleotide to a subject or a cell. In certain embodiments, a prophylactically effective amount is an amount sufficient for delivering mRNA to a subject or a cell. In certain embodiments, a prophylactically effective amount is an amount sufficient for preventing a disease, disorder, or condition. In certain embodiments, a prophylactically effective amount is an amount sufficient for delivering an agent to a subject or a cell and preventing a disease, disorder, or condition. In certain embodiments, a prophylactically effective amount is an amount sufficient for delivering a polynucleotide to a subject or a cell and preventing a disease, disorder, or condition. In certain embodiments, a prophylactically effective amount is an amount sufficient for delivering mRNA to a subject or a cell and preventing a disease, disorder, or condition. In certain embodiments, a prophylactically effective amount is an amount effective for chimeric antigen receptor T cell therapy.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstram's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculoskeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g., nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

In certain embodiments, the painful condition is neuropathic pain. The term “neuropathic pain” refers to pain resulting from injury to a nerve. Neuropathic pain is distinguished from nociceptive pain, which is the pain caused by acute tissue injury involving small cutaneous nerves or small nerves in muscle or connective tissue. Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Neuropathic pain conditions can develop following neuronal injury and the resulting pain may persist for months or years, even after the original injury has healed. Neuronal injury may occur in the peripheral nerves, dorsal roots, spinal cord or certain regions in the brain. Neuropathic pain conditions include, but are not limited to, diabetic neuropathy (e.g., peripheral diabetic neuropathy); sciatica; non-specific lower back pain; multiple sclerosis pain; carpal tunnel syndrome, fibromyalgia; HIV-related neuropathy; neuralgia (e.g., post-herpetic neuralgia, trigeminal neuralgia); pain resulting from physical trauma (e.g., amputation; surgery, invasive medical procedures, toxins, burns, infection), pain resulting from cancer or chemotherapy (e.g., chemotherapy-induced pain such as chemotherapy-induced peripheral neuropathy), and pain resulting from an inflammatory condition (e.g., a chronic inflammatory condition). Neuropathic pain can result from a peripheral nerve disorder such as neuroma; nerve compression; nerve crush, nerve stretch or incomplete nerve transsection; mononeuropathy or polyneuropathy. Neuropathic pain can also result from a disorder such as dorsal root ganglion compression; inflammation of the spinal cord; contusion, tumor or hemisection of the spinal cord; tumors of the brainstem, thalamus or cortex; or trauma to the brainstem, thalamus or cortex.

The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia). [00449] In certain embodiments, the painful condition is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulus of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “musculoskeletal disease” or “MSD” refers to an injury and/or pain in a subject's joints, ligaments, muscles, nerves, tendons, and structures that support limbs, neck, and back. In certain embodiments, an MSD is a degenerative disease. In certain embodiments, an MSD includes an inflammatory condition. Body parts of a subject that may be associated with MSDs include upper and lower back, neck, shoulders, and extremities (arms, legs, feet, and hands). In certain embodiments, an MSD is a bone disease, such as achondroplasia, acromegaly, bone callus, bone demineralization, bone fracture, bone marrow disease, bone marrow neoplasm, dyskeratosis congenita, leukemia (e.g., hairy cell leukemia, lymphocytic leukemia, myeloid leukemia, Philadelphia chromosome-positive leukemia, plasma cell leukemia, stem cell leukemia), systemic mastocytosis, myelodysplastic syndromes, paroxysmal nocturnal hemoglobinuria, myeloid sarcoma, myeloproliferative disorders, multiple myeloma, polycythemia vera, pearson marrow-pancreas syndrome, bone neoplasm, bone marrow neoplasm, Ewing sarcoma, osteochondroma, osteoclastoma, osteosarcoma, brachydactyly, Camurati-Engelmann syndrome, Craniosynostosis, Crouzon craniofacial dysostosis, dwarfism, achondroplasia, bloom syndrome, Cockayne syndrome, Ellis-van Creveld syndrome, Seckel syndrome, spondyloepiphyseal dysplasia, spondyloepiphyseal dysplasia congenita, Werner syndrome, hyperostosis, osteophyte, Klippel-Trenaunay-Weber syndrome, Marfan syndrome, McCune-Albright syndrome, osteitis, osteoarthritis, osteochondritis, osteochondrodysplasia, Kashin-Beck disease, Leri-Weill dyschondrosteosis, osteochondrosis, osteodystrophy, osteogenesis imperfecta, osteolysis, Gorham-Stout syndrome, osteomalacia, osteomyelitis, osteonecrosis, osteopenia, osteopetrosis, osteoporosis, osteosclerosis, otospondylomegaepiphyseal dysplasia, pachydermoperiostosis, Paget disease of bone, Polydactyly, Meckel syndrome, rickets, Rothmund-Thomson syndrome, Sotos syndrome, spondyloepiphyseal dysplasia, spondyloepiphyseal dysplasia congenita, syndactyly, Apert syndrome, syndactyly type II, or Werner syndrome. In certain embodiments, an MSD is a cartilage disease, such as cartilage neoplasm, osteochondritis, osteochondrodysplasia, Kashin-Beck disease, or Leri-Weill dyschondrosteosis. In certain embodiments, an MSD is hernia, such as intervertebral disk hernia. In certain embodiments, an MSD is a joint disease, such as arthralgia, arthritis (e.g., gout (e.g., Kelley-Seegmiller syndrome, Lesch-Nyhan syndrome), Lyme disease, osteoarthritis, psoriatic arthritis, reactive arthritis, rheumatic fever, rheumatoid arthritis, Felty syndrome, synovitis, Blau syndrome, nail-patella syndrome, spondyloarthropathy, reactive arthritis, Stickler syndrome, synovial membrane disease, synovitis, or Blau syndrome. In certain embodiments, an MSD is Langer-Giedion syndrome. In certain embodiments, an MSD is a muscle disease, such as Barth syndrome, mitochondrial encephalomyopathy, MELAS syndrome, MERRF syndrome, MNGIE syndrome, mitochondrial myopathy, Kearns-Sayre syndrome, myalgia, fibromyalgia, polymyalgia rheumatica, myoma, myositis, dermatomyositis, neuromuscular disease, Kearns-Sayre syndrome, muscular dystrophy, myasthenia, congenital myasthenic syndrome, Lambert-Eaton myasthenic syndrome, myasthenia gravis, myotonia, myotonia congenita, spinal muscular atrophy, tetany, ophthalmoplegia, or rhabdomyolysis. In certain embodiments, an MSD is Proteus syndrome. In certain embodiments, an MSD is a rheumatic diseases, such as arthritis (e.g., gout (e.g., Kelley-Seegmiller syndrome, Lesch-Nyhan lyme disease)), osteoarthritis, psoriatic arthritis, reactive arthritis, rheumatic fever, rheumatoid arthritis, Felty syndrome, synovitis, Blau syndrome, gout (e.g., Kelley-Seegmiller syndrome, Lesch-Nyhan syndrome), polymyalgia rheumatica, rheumatic fever, rheumatic heart disease, or Sjogren syndrome. In certain embodiments, an MSD is Schwartz-Jampel syndrome. In certain embodiments, an MSD is a skeleton disease, such as Leri-Weill dyschondrosteosis, skeleton malformations, Melnick-Needles syndrome, pachydermoperiostosis, Rieger syndrome, spinal column disease, intervertebral disk hernia, scoliosis, spina bifida, spondylitis, ankylosing spondylitis, spondyloarthropathy, reactive arthritis, spondyloepiphyseal dysplasia, spondyloepiphyseal dysplasia congenita, or spondylosis.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

In certain embodiments, the metabolic disorder is a wasting condition. A “wasting condition” includes but is not limited to, anorexia and cachexias of various natures (e.g., weight loss associated with cancer, weight loss associated with other general medical conditions, weight loss associated with failure to thrive, and the like). In certain embodiments, the metabolic disorder is an obesity-related condition or a complication thereof. An “obesity-related condition” includes, but is not limited to, obesity, undesired weight gain (e.g., from medication-induced weight gain, from cessation of smoking) and an over-eating disorder (e.g., binge eating, bulimia, compulsive eating, or a lack of appetite control each of which can optionally lead to undesired weight gain or obesity). “Obesity” and “obese” refers to class I obesity, class II obesity, class III obesity and pre-obesity (e.g., being “over-weight”) as defined by the World Health Organization.

Reduction of storage fat is expected to provide various primary and/or secondary benefits in a subject (e.g., in a subject diagnosed with a complication associated with obesity) such as, for example, an increased insulin responsiveness (e.g., in a subject diagnosed with Type II diabetes mellitus); a reduction in elevated blood pressure; a reduction in elevated cholesterol levels; and/or a reduction (or a reduced risk or progression) of ischemic heart disease, arterial vascular disease, angina, myocardial infarction, stroke, migraines, congestive heart failure, deep vein thrombosis, pulmonary embolism, gall stones, gastroesophagael reflux disease, obstructive sleep apnea, obesity hypoventilation syndrome, asthma, gout, poor mobility, back pain, erectile dysfunction, urinary incontinence, liver injury (e.g., fatty liver disease, liver cirrhosis, alcoholic cirrhosis, endotoxin mediated liver injury) or chronic renal failure.

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease).

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

In certain embodiments, the inflammatory disorder and/or the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD).

In certain embodiments, the inflammatory condition and/or immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein are compounds (e.g., compounds of Formula (I)), and pharmaceutically acceptable salts thereof, and compositions and kits thereof. The compounds provided herein can form particles and may therefore be used to deliver agents (e.g., a polynucleotide) to a subject, target tissue, or cell. Also provided herein are methods of delivery and methods of treating a disease, disorder, or condition, comprising administering to the subject a composition comprising a compound provided herein (e.g., a compound of Formula (I)), or a pharmaceutically acceptable salt thereof.

### Compounds

Provided herein are compounds of Formula (IA):

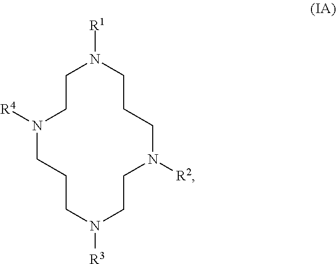

or a pharmaceutically acceptable salt thereof,
wherein:
each of R¹, R², R³, and R⁴ is independently hydrogen, a nitrogen protecting group, or a group of the formula (i), (ii), (iii), (iv) or (v):

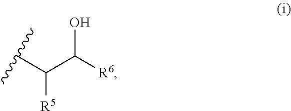

wherein one of R⁶ and R⁷ is hydrogen, and the other is

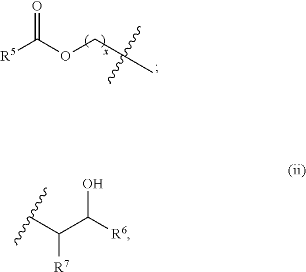

wherein one of R⁶ and R⁷ is hydrogen, and the other is

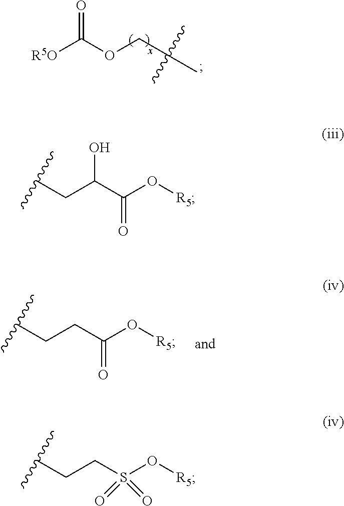

wherein
each instance of R⁵ is independently optionally substituted C₄-C₂₅ saturated or unsaturated aliphatic or optionally substituted C₄-C₂₅ saturated or unsaturated heteroaliphatic; and
each x independently is an integer from 1 to 10, inclusive;
provided that at least one of R¹, R², R³, and R⁴ is formula (i), (ii), (iii), (iv), or (v).

In some embodiments of Formula (IA), each of R¹, R², R³, and R⁴ is independently hydrogen, a nitrogen protecting group, or a group of the formula (i), provided that at least one of R¹, R², R³, and R⁴ is formula (i).

In some embodiments of Formula (IA), each of R¹, R², R³, and R⁴ is independently hydrogen, a nitrogen protecting group, or a group of the formula (ii), provided that at least one of R¹, R², R³, and R⁴ is formula (ii).

In some embodiments of Formula (IA), each of R¹, R², R³, and R⁴ is independently hydrogen, a nitrogen protecting group, or a group of the formula (iii), provided that at least one of R¹, R², R³, and R⁴ is formula (iii).

In some embodiments of Formula (IA), each of R¹, R², R³, and R⁴ is independently hydrogen, a nitrogen protecting group, or a group of the formula (iv), provided that at least one of R¹, R², R³, and R⁴ is formula (iv).

In some embodiments of Formula (IA), each of R¹, R², R³, and R⁴ is independently hydrogen, a nitrogen protecting group, or a group of the formula (v), provided that at least one of R¹, R², R³, and R⁴ is formula (v).

Provided herein are compounds of Formula (I):

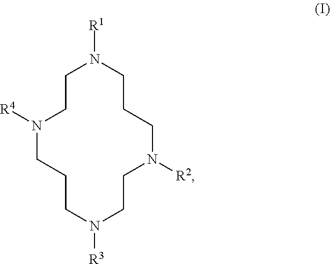

or a pharmaceutically acceptable salt thereof,
wherein:
each of R¹, R², R³, and R⁴ is independently hydrogen, a nitrogen protecting group, or

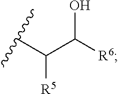

one of R⁶ and R⁷ is hydrogen, and the other is

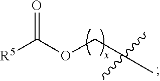

each instance of R⁵ is independently optionally substituted C₄-C₂₅ saturated or unsaturated aliphatic or optionally substituted C₄-C₂₅ saturated or unsaturated heteroaliphatic; and
each x independently is an integer from 1 to 10, inclusive;
provided that at least one of R¹, R², R³, and R⁴ is

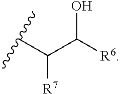

As defined herein, each of R¹, R², R³, and R⁴ is independently hydrogen, a nitrogen protecting group, or

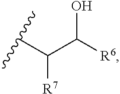

provided that least one of R¹, R², R³, and R⁴ is

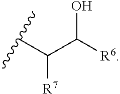

In some embodiments, at least two of R¹, R², R³, and R⁴ is independently

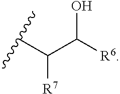

In some embodiments, at least three of R¹, R², R³, and R⁴ is independently

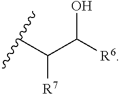

In some embodiments, each of R¹, R², R³, and R⁴ is independently

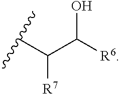

In some embodiments, R¹ is

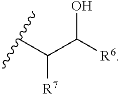

In some embodiments, R² is

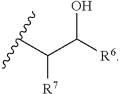

In some embodiments, R³ is

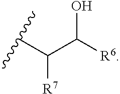

In some embodiments, R⁴ is

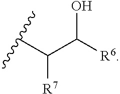

In some embodiments, R¹ and R² are independently

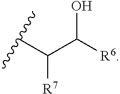

In some embodiments, R¹ and R³ are independently

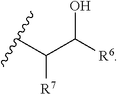

In some embodiments, R¹ and R⁴ are independently

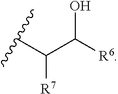

In some embodiments, R² and R³ are independently

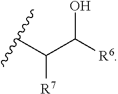

In some embodiments, R² and R⁴ are independently

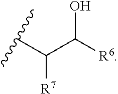

In some embodiments, R³ and R⁴ are independently

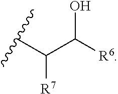

In some embodiments, R¹, R², and R³ are independently

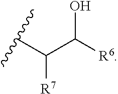

In some embodiments, R¹, R², and R⁴ are independently

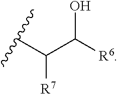

In some embodiments, R¹, R³, and R⁴ are independently

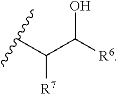

In some embodiments, R², R³, and R⁴ are independently

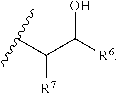

In some embodiments, R¹ is hydrogen, a nitrogen protecting group, or

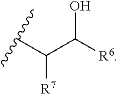

In some embodiments, R¹ is hydrogen or a nitrogen protecting group. In some embodiments, R¹ is hydrogen or

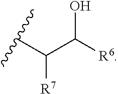

In some embodiments, R¹ is a nitrogen protecting group or

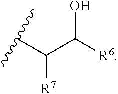

In some embodiments, R¹ is hydrogen. In some embodiments, R¹ is a nitrogen protecting group. In some embodiments, R¹ is Bn, Boc, Cbz, Fmoc, trifluoroacetyl, triphenylmethyl, acetyl, or Ts. In some embodiments, R¹ is

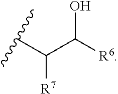

In some embodiments, R² is hydrogen, a nitrogen protecting group, or

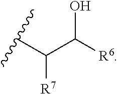

In some embodiments, R² is hydrogen or a nitrogen protecting group. In some embodiments, R² is hydrogen or

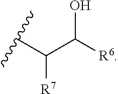

In some embodiments, R² is a nitrogen protecting group or

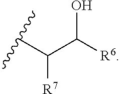

In some embodiments, R² is hydrogen. In some embodiments, R² is a nitrogen protecting group. In some embodiments, R² is Bn, Boc, Cbz, Fmoc, trifluoroacetyl, triphenylmethyl, acetyl, or Ts. In some embodiments, R² is

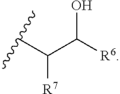

In some embodiments, R³ is hydrogen, a nitrogen protecting group, or

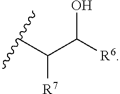

In some embodiments, R³ is hydrogen or a nitrogen protecting group. In some embodiments, R³ is hydrogen or

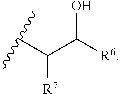

In some embodiments, R³ is a nitrogen protecting group or

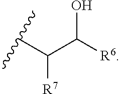

In some embodiments, R³ is hydrogen. In some embodiments, R³ is a nitrogen protecting group. In some embodiments, R³ is Bn, Boc, Cbz, Fmoc, trifluoroacetyl, triphenylmethyl, acetyl, or Ts. In some embodiments, R³ is

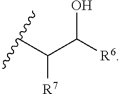

In some embodiments, R⁴ is hydrogen, a nitrogen protecting group, or

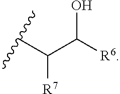

In some embodiments, R⁴ is hydrogen or a nitrogen protecting group. In some embodiments, R⁴ is hydrogen or

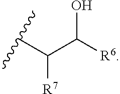

In some embodiments, R⁴ is a nitrogen protecting group or

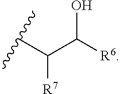

In some embodiments, R⁴ is hydrogen. In some embodiments, R⁴ is a nitrogen protecting group. In some embodiments, R⁴ is Bn, Boc, Cbz, Fmoc, trifluoroacetyl, triphenylmethyl, acetyl, or Ts. In some embodiments, R⁴ is

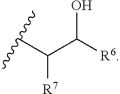

As defined herein, for each moiety

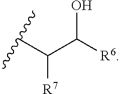

individually, one of R⁶ and R⁷ is hydrogen, and the other is

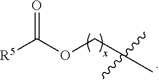

In some embodiments, R⁶ is hydrogen. In some embodiments, R⁷ is hydrogen. In some embodiments, R⁶ is

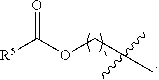

In some embodiments, R⁷ is

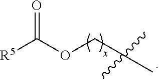

In some embodiments, each instance of R⁵ is different. In some embodiments, two instances of R⁵ are the same. In some embodiments, at least two instances of R⁵ are the same. In some embodiments, three instances of R⁵ are the same. In some embodiments, at least three instances of R⁵ are the same. In some embodiments, each instance of R⁵ is the same.

In some embodiments, each instance of R⁵ is independently optionally substituted C₄-C₂₅ saturated aliphatic or optionally substituted C₄-C₂₅ saturated heteroaliphatic. In some embodiments, each instance of R⁵ is independently optionally substituted C₄-C₂₅ unsaturated aliphatic or optionally substituted C₄-C₂₅ unsaturated heteroaliphatic.

In some embodiments, each instance of R⁵ is independently optionally substituted C₄-C₂₅ saturated or unsaturated aliphatic. In some embodiments, each instance of R⁵ is independently optionally substituted C₄-C₁₂ saturated or unsaturated aliphatic. In some embodiments, each instance of R⁵ is independently optionally substituted C₁₀-C₂₀ saturated or unsaturated aliphatic. In some embodiments, each instance of R⁵ is independently optionally substituted C₁₂-C₂₅ saturated or unsaturated aliphatic.

In some embodiments, each R⁵ is independently optionally substituted C₄-C₂₅ alkyl or optionally substituted C₄-C₂₅ alkenyl. In some embodiments, each R⁵ is independently optionally substituted C₄-C₁₀ alkyl or optionally substituted C₄-C₁₀ alkenyl. In some embodiments, each R⁵ is independently optionally substituted C₁₀-C₂₀ alkyl or optionally substituted C₁₀-C₂₀ alkenyl. In some embodiments, each R⁵ is independently optionally substituted C₁₂-C₂₅ alkyl or optionally substituted C₁₂-C₂₅ alkenyl.

In some embodiments, each R⁵ is independently optionally substituted C₄-C₂₅ alkyl or optionally substituted C₄-C₂₅ alkynyl. In some embodiments, each R⁵ is independently optionally substituted C₄-C₁₀ alkyl or optionally substituted C₄-C₁₀ alkynyl. In some embodiments, each R⁵ is independently optionally substituted C₁₀-C₂₀ alkyl or optionally substituted C₁₀-C₂₀ alkynyl. In some embodiments, each R⁵ is independently optionally substituted C₁₂-C₂₅ alkyl or optionally substituted C₁₂-C₂₅ alkynyl.

In some embodiments, each R⁵ is independently optionally substituted C₄-C₂₅ alkynyl or optionally substituted C₄-C₂₅ alkenyl. In some embodiments, each R⁵ is independently optionally substituted C₄-C₁₀ alkynyl or optionally substituted C₄-C₁₀ alkenyl. In some embodiments, each R⁵ is independently optionally substituted C₁₀-C₂₀ alkynyl or optionally substituted C₁₀-C₂₀ alkenyl. In some embodiments, each R⁵ is independently optionally substituted C₁₂-C₂₅ alkynyl or optionally substituted C₁₂-C₂₅ alkenyl.

In some embodiments, each R⁵ is independently branched C₄-C₂₅ alkyl. In some embodiments, each R⁵ is independently branched C₄-C₁₂ alkyl. In some embodiments, each R⁵ is independently branched C₁₀-C₂₀ alkyl. In some embodiments, each R⁵ is independently branched C₁₂-C₂₅ alkyl. In some embodiments, each R⁵ is independently unbranched C₄-C₂₅ alkyl. In some embodiments, each R⁵ is independently unbranched C₄-C₁₂ alkyl. In some embodiments, each R⁵ is independently unbranched C₁₀-C₂₀ alkyl. In some embodiments, each R⁵ is independently unbranched C₁₂-C₂₅ alkyl.

In some embodiments, each R⁵ is independently branched C₄-C₂₅ alkenyl. In some embodiments, each R⁵ is independently branched C₄-C₁₂ alkenyl. In some embodiments, each R⁵ is independently branched C₁₀-C₂₀ alkenyl. In some embodiments, each R⁵ is independently branched C₁₂-C₂₅ alkenyl. In some embodiments, each R⁵ is independently unbranched C₄-C₂₅ alkenyl. In some embodiments, each R⁵ is independently unbranched C₄-C₁₂ alkenyl. In some embodiments, each R⁵ is independently unbranched C₁₀-C₂₀ alkenyl. In some embodiments, each R⁵ is independently unbranched C₁₂-C₂₅ alkenyl.

In some embodiments, each R⁵ is unbranched C₁₁ alkenyl. In some embodiments, the C₁₁ alkenyl comprises a single double bond. In some embodiments, the C₁₁ alkenyl comprises double bonds. In some embodiments, the C₁₁ alkenyl comprises three double bonds. In some embodiments, the C₁₁ alkenyl comprises only (Z) double bonds.

In some embodiments, each R⁵ is independently branched C₄-C₂₅ alkynyl. In some embodiments, each R⁵ is independently branched C₄-C₁₂ alkynyl. In some embodiments, R⁵ is independently branched C₁₀-C₂₀ alkynyl. In some embodiments, each R⁵ is independently branched C₁₂-C₂₅ alkynyl. In some embodiments, each R⁵ is independently unbranched C₄-C₂₅ alkynyl. In some embodiments, each R⁵ is independently unbranched C₄-C₁₂ alkynyl. In some embodiments, each R⁵ is independently unbranched C₁₀-C₂₀ alkynyl. In some embodiments, each R⁵ is independently unbranched C₁₂-C₂₅ alkynyl.

In some embodiments, each instance of R⁵ is independently optionally substituted C₄-C₂₅ saturated or unsaturated heteroaliphatic. In some embodiments, each instance of R⁵ is independently optionally substituted C₄-C₂₅ saturated or unsaturated heteroaliphatic comprising at least one O, N, or S atom. In some embodiments, each instance of R⁵ is independently optionally substituted C₄-C₂₅ saturated or unsaturated heteroaliphatic comprising at least one O or S atom. In some embodiments, each instance of R⁵ is independently optionally substituted C₄-C₁₂ saturated or unsaturated heteroaliphatic. In some embodiments, each instance of R⁵ is independently optionally substituted C₄-C₁₂ saturated or unsaturated heteroaliphatic comprising at least one O, N, or S atom. In some embodiments, each instance of R⁵ is independently optionally substituted C₄-C₁₂ saturated or unsaturated heteroaliphatic comprising at least one O or S atom. In some embodiments, each instance of R⁵ is independently optionally substituted C₁₀-C₂₀ saturated or unsaturated heteroaliphatic. In some embodiments, each instance of R⁵ is independently optionally substituted C₁₀-C₂₀ saturated or unsaturated heteroaliphatic comprising at least one O, N, or S atom. In some embodiments, each instance of R⁵ is independently optionally substituted C₁₀-C₂₀ saturated or unsaturated heteroaliphatic comprising at least one O or S atom. In some embodiments, each instance of R⁵ is independently optionally substituted C₁₂-C₂₅ saturated or unsaturated heteroaliphatic. In some embodiments, each instance of R⁵ is independently optionally substituted C₁₂-C₂₅ saturated or unsaturated heteroaliphatic comprising at least one O, N, or S atom. In some embodiments, each instance of R⁵ is independently optionally substituted C₁₂-C₂₅ saturated or unsaturated heteroaliphatic comprising at least one O or S atom.

In some embodiments, each R⁵ is independently optionally substituted C₄-C₂₅ heteroalkyl or optionally substituted C₄-C₂₅ heteroalkenyl. In some embodiments, each R⁵ is independently optionally substituted C₄-C₁₀ heteroalkyl or optionally substituted C₄-C₁₀ heteroalkenyl. In some embodiments, each R⁵ is independently optionally substituted C₁₀-C₂₀ heteroalkyl or optionally substituted C₁₀-C₂₀ heteroalkenyl. In some embodiments, each R⁵ is independently optionally substituted C₁₂-C₂₅ heteroalkyl or optionally substituted C₁₂-C₂₅ heteroalkenyl.

In some embodiments, each R⁵ is independently optionally substituted C₄-C₂₅ heteroalkyl or optionally substituted C₄-C₂₅ heteroalkynyl. In some embodiments, each R⁵ is independently optionally substituted C₄-C₁₀ heteroalkyl or optionally substituted C₄-C₁₀ heteroalkynyl. In some embodiments, each R⁵ is independently optionally substituted C₁₀-C₂₀ heteroalkyl or optionally substituted C₁₀-C₂₀ heteroalkynyl. In some embodiments, each R⁵ is independently optionally substituted C₁₂-C₂₅ heteroalkyl or optionally substituted C₁₂-C₂₅ heteroalkynyl.

In some embodiments, each R⁵ is independently optionally substituted C₄-C₂₅ heteroalkynyl or optionally substituted C₄-C₂₅ heteroalkenyl. In some embodiments, each R⁵ is independently optionally substituted C₄-C₁₀ heteroalkynyl or optionally substituted C₄-C₁₀ heteroalkenyl. In some embodiments, each R⁵ is independently optionally substituted C₁₀-C₂₀ heteroalkynyl or optionally substituted C₁₀-C₂₀ heteroalkenyl. In some embodiments, each R⁵ is independently optionally substituted C₁₂-C₂₅ heteroalkynyl or optionally substituted C₁₂-C₂₅ heteroalkenyl.

In some embodiments, each R⁵ is independently branched C₄-C₂₅ heteroalkyl. In some embodiments, R⁵ is independently branched C₄-C₁₂ heteroalkyl. In some embodiments, each R⁵ is independently branched C₁₀-C₂₀ heteroalkyl. In some embodiments, each R⁵ is independently branched C₁₂-C₂₅ heteroalkyl. In some embodiments, each R⁵ is independently unbranched C₄-C₂₅ heteroalkyl. In some embodiments, each R⁵ is independently unbranched C₄-C₁₂ heteroalkyl. In some embodiments, each R⁵ is independently unbranched C₁₀-C₂₀ heteroalkyl. In some embodiments, each R⁵ is independently unbranched C₁₂-C₂₅ heteroalkyl.

In some embodiments, each R⁵ is independently branched C₄-C₂₅ heteroalkenyl. In some embodiments, each R⁵ is independently branched C₄-C₁₂ heteroalkenyl. In some embodiments, each R⁵ is independently branched C₁₀-C₂₀ heteroalkenyl. In some embodiments, each R⁵ is independently branched C₁₂-C₂₅ heteroalkenyl. In some embodiments, each R⁵ is independently unbranched C₄-C₂₅ heteroalkenyl. In some embodiments, each R⁵ is independently unbranched C₄-C₁₂ heteroalkenyl. In some embodiments, each R⁵ is independently unbranched C₁₀-C₂₀ heteroalkenyl. In some embodiments, each R⁵ is independently unbranched C₁₂-C₂₅ heteroalkenyl.

In some embodiments, each R⁵ is independently branched C₄-C₂₅ heteroalkynyl. In some embodiments, each R⁵ is independently branched C₄-C₁₂ heteroalkynyl. In some embodiments, each R⁵ is independently branched C₁₀-C₂₀ heteroalkynyl. In some embodiments, each R⁵ is independently branched C₁₂-C₂₅ heteroalkynyl. In some embodiments, each R⁵ is independently unbranched C₄-C₂₅ heteroalkynyl. In some embodiments, each R⁵ is independently unbranched C₄-C₁₂ heteroalkynyl. In some embodiments, each R⁵ is independently unbranched C₁₀-C₂₀ heteroalkynyl. In some embodiments, each R⁵ is independently unbranched C₁₂-C₂₅ heteroalkynyl.

In some embodiments, each R⁵ is independently selected from the group consisting of:

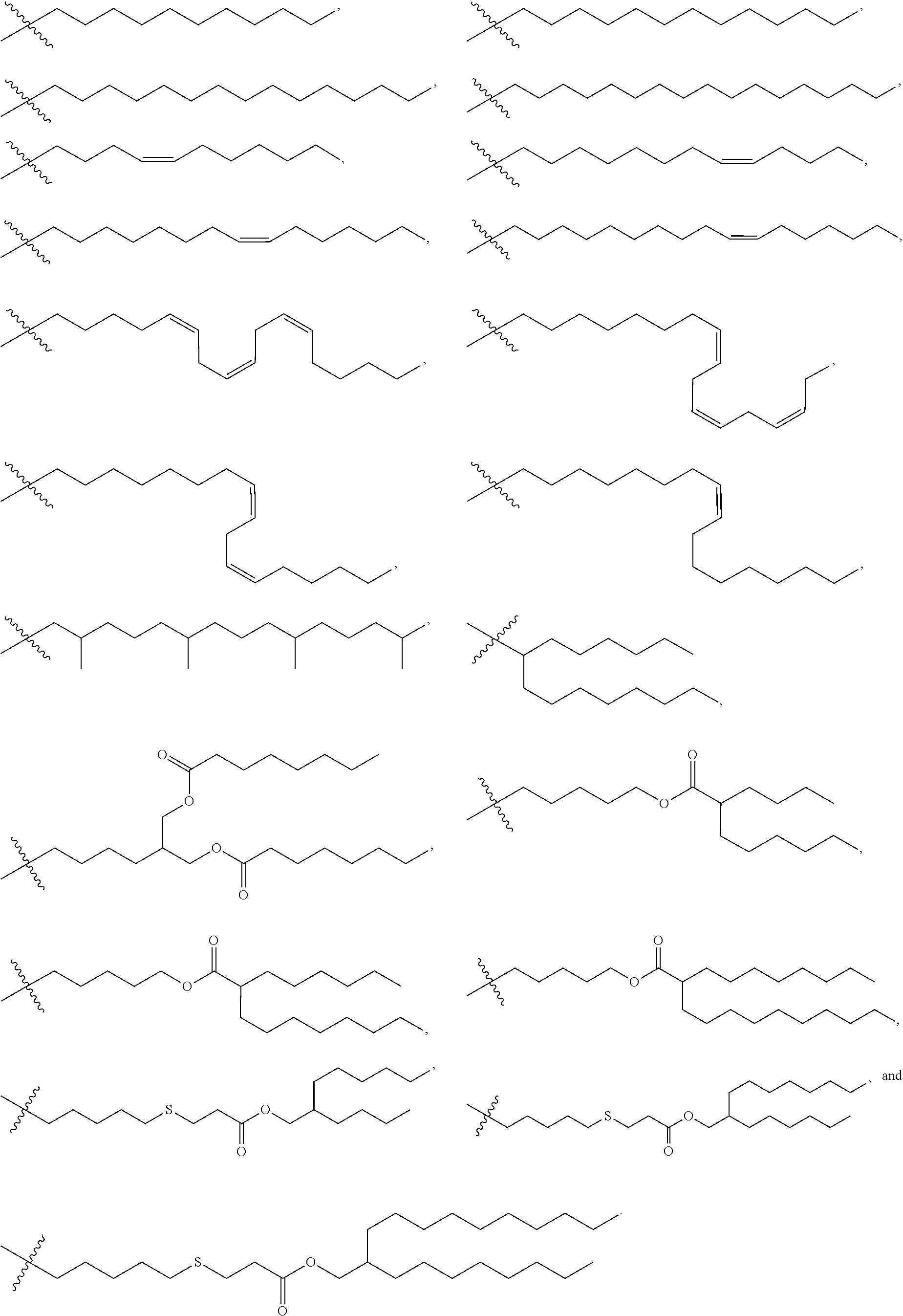

In some embodiments, each R⁵ is independently selected from the group consisting of:

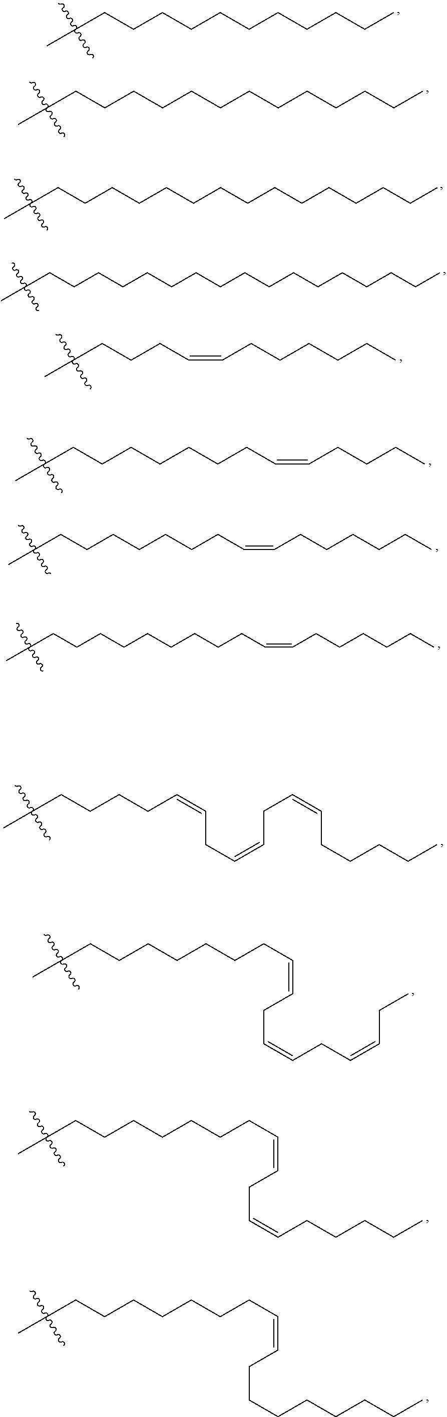

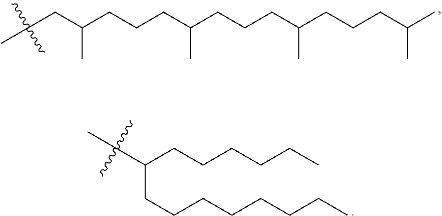 [SMILES: *CC(C)CCCC(C)CCCC(C)CCCC(C)C ; "," ; *C(CCCCCC)CCCCCCCC ; "."]

In some embodiments, each R⁵ is independently selected from the group consisting of:

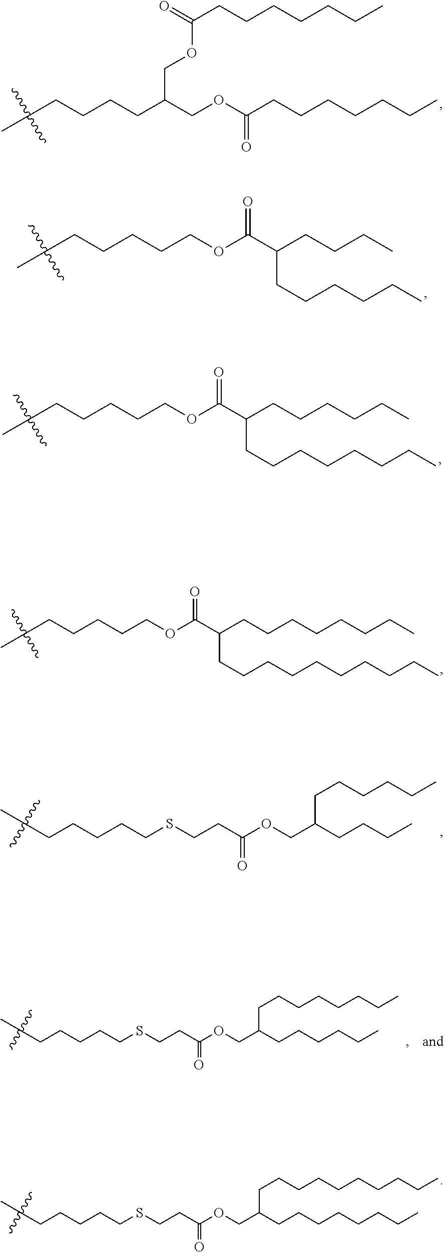

In some embodiments, each R⁵ is

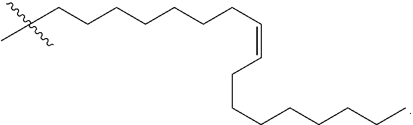 [SMILES: *CCCCCCC/C=C\CCCCCCCC]

In some embodiments each R⁵ is independently selected from the group consisting of:

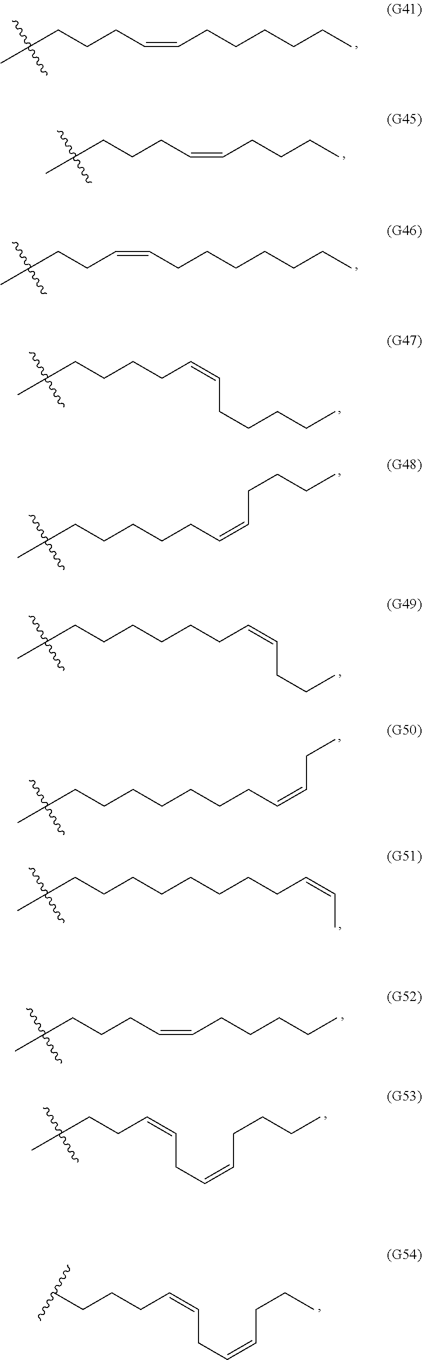

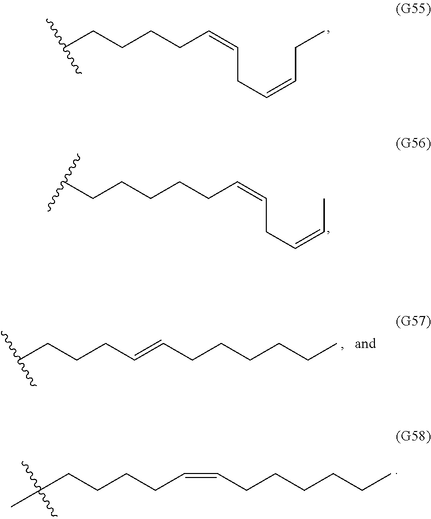

In some embodiments, each R⁵ is independently selected from the group consisting of:

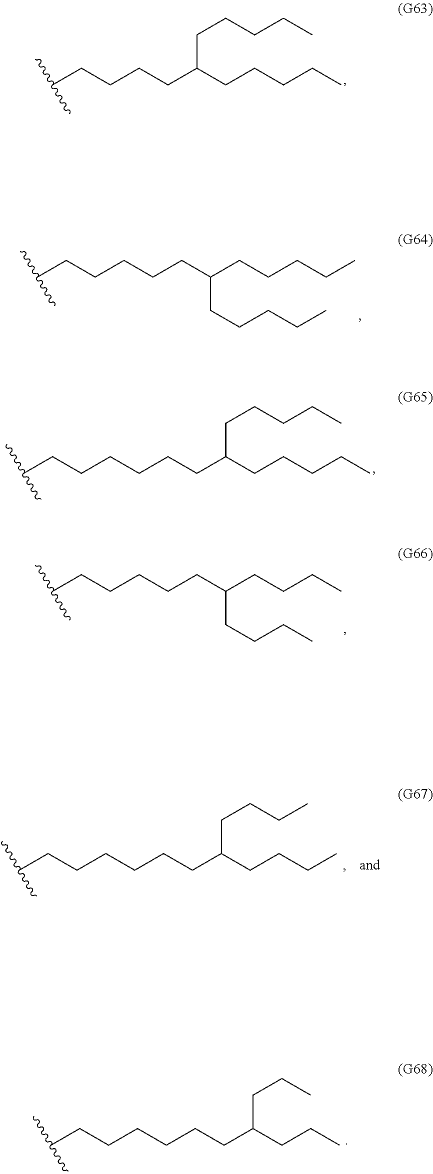 [SMILES: *CCCCC(CCCCC)CCCCC ; *CCCCCC(CCCCC)CCCCC ; *CCCCCCC(CCCCC)CCCCC ; *CCCCCC(CCCC)CCCC ; *CCCCCCC(CCCC)CCCC ; *CCCCCCC(CCC)CCC]

In a particular embodiment, each R⁵ is G41. In a particular embodiment, each R⁵ is G45. In a particular embodiment, each R⁵ is G46. In a particular embodiment, each R⁵ is G47. In a particular embodiment, each R⁵ is G48. In a particular embodiment, each R⁵ is G49. In a particular embodiment, each R⁵ is G50. In a particular embodiment, each R⁵ is G51. In a particular embodiment, each R⁵ is G52. In a particular embodiment, each R⁵ is G53. In a particular embodiment, each R⁵ is G54. In a particular embodiment, each R⁵ is G55. In a particular embodiment, each R⁵ is G56. In a particular embodiment, each R⁵ is G57. In a particular embodiment, each R⁵ is G58. In a particular embodiment, each R⁵ is G63. In a particular embodiment, each R⁵ is G64. In a particular embodiment, each R⁵ is G65. In a particular embodiment, each R⁵ is G66. In a particular embodiment, each R⁵ is G67. In a particular embodiment, each R⁵ is G68.

In some embodiments, at least one instance of R⁵ is independently optionally substituted C₄-C₂₅ saturated or unsaturated aliphatic. In some embodiments, at least one instance of R⁵ is independently optionally substituted C₄-C₁₂ saturated or unsaturated aliphatic. In some embodiments, at least one instance of R⁵ is independently optionally substituted C₁₀-C₂₀ saturated or unsaturated aliphatic. In some embodiments, at least one instance of R⁵ is independently optionally substituted C₁₂-C₂₅ saturated or unsaturated aliphatic.

In some embodiments, at least one R⁵ is independently optionally substituted C₄-C₂₅ alkyl or optionally substituted C₄-C₂₅ alkenyl. In some embodiments, at least one R⁵ is independently optionally substituted C₄-C₁₀ alkyl or optionally substituted C₄-C₁₀ alkenyl. In some embodiments, at least one R⁵ is independently optionally substituted C₁₀-C₂₀ alkyl or optionally substituted C₁₀-C₂₀ alkenyl. In some embodiments, at least one R⁵ is independently optionally substituted C₁₂-C₂₅ alkyl or optionally substituted C₁₂-C₂₅ alkenyl.

In some embodiments, at least one R⁵ is independently optionally substituted C₄-C₂₅ alkyl or optionally substituted C₄-C₂₅ alkynyl. In some embodiments, at least one R⁵ is independently optionally substituted C₄-C₁₀ alkyl or optionally substituted C₄-C₁₀ alkynyl. In some embodiments, at least one R⁵ is independently optionally substituted C₁₀-C₂₀ alkyl or optionally substituted C₁₀-C₂₀ alkynyl. In some embodiments, at least one R⁵ is independently optionally substituted C₁₂-C₂₅ alkyl or optionally substituted C₁₂-C₂₅ alkynyl.

In some embodiments, at least one R⁵ is independently optionally substituted C₄-C₂₅ alkynyl or optionally substituted C₄-C₂₅ alkenyl. In some embodiments, at least one R⁵ is independently optionally substituted C₄-C₁₀ alkynyl or optionally substituted C₄-C₁₀ alkenyl. In some embodiments, at least one R⁵ is independently optionally substituted C₁₀-C₂₀ alkynyl or optionally substituted C₁₀-C₂₀ alkenyl. In some embodiments, at least one R⁵ is independently optionally substituted C₁₂-C₂₅ alkynyl or optionally substituted C₁₂-C₂₅ alkenyl.

In some embodiments, at least one R⁵ is unbranched C₁₁ alkenyl. In some embodiments, the C₁₁ alkenyl comprises a single double bond. In some embodiments, the C₁₁ alkenyl comprises double bonds. In some embodiments, the C₁₁ alkenyl comprises three double bonds. In some embodiments, the C₁₁ alkenyl comprises only (Z) double bonds.

In some embodiments, at least one R⁵ is independently branched C₄-C₂₅ alkyl. In some embodiments, at least one R⁵ is independently branched C₄-C₁₂ alkyl. In some embodiments, at least one R⁵ is independently branched C₁₀-C₂₀ alkyl. In some embodiments, at least one R⁵ is independently branched C₁₂-C₂₅ alkyl. In some embodiments, at least one R⁵ is independently unbranched C₄-C₂₅ alkyl. In some embodiments, at least one R⁵ is independently unbranched C₄-C₁₂ alkyl. In some embodiments, at least one R⁵ is independently unbranched C₁₀-C₂₀ alkyl. In some embodiments, at least one R⁵ is independently unbranched C₁₂-C₂₅ alkyl.

In some embodiments, at least one R⁵ is independently branched C₄-C₂₅ alkenyl. In some embodiments, at least one R⁵ is independently branched C₄-C₁₂ alkenyl. In some embodiments, at least one R⁵ is independently branched C₁₀-C₂₀ alkenyl. In some embodiments, at least one R⁵ is independently branched C₁₂-C₂₅ alkenyl. In some embodiments, at least one R⁵ is independently unbranched C₄-C₂₅ alkenyl. In some embodiments, at least one R⁵ is independently unbranched C₄-C₁₂ alkenyl. In some embodiments, at least one R⁵ is independently unbranched C₁₀-C₂₀ alkenyl. In some embodiments, at least one R⁵ is independently unbranched C₁₂-C₂₅ alkenyl.

In some embodiments, at least one R⁵ is independently branched C₄-C₂₅ alkynyl. In some embodiments, at least one R⁵ is independently branched C₄-C₁₂ alkynyl. In some embodiments, R⁵ is independently branched C₁₀-C₂₀ alkynyl. In some embodiments, at least one R⁵ is independently branched C₁₂-C₂₅ alkynyl. In some embodiments, at least one R⁵ is independently unbranched C₄-C₂₅ alkynyl. In some embodiments, at least one R⁵ is independently unbranched C₄-C₁₂ alkynyl. In some embodiments, at least one R⁵ is independently unbranched C₁₀-C₂₀ alkynyl. In some embodiments, at least one R⁵ is independently unbranched C₁₂-C₂₅ alkynyl.

In some embodiments, at least one instance of R⁵ is independently optionally substituted C₄-C₂₅ saturated or unsaturated heteroaliphatic. In some embodiments, at least one instance of R⁵ is independently optionally substituted C₄-C₂₅ saturated or unsaturated heteroaliphatic comprising at least one O, N, or S atom. In some embodiments, at least one instance of R⁵ is independently optionally substituted C₄-C₂₅ saturated or unsaturated heteroaliphatic comprising at least one O or S atom. In some embodiments, at least one instance of R⁵ is independently optionally substituted C₄-C₁₂ saturated or unsaturated heteroaliphatic. In some embodiments, at least one instance of R⁵ is independently optionally substituted C₄-C₁₂ saturated or unsaturated heteroaliphatic comprising at least one O, N, or S atom. In some embodiments, at least one instance of R⁵ is independently optionally substituted C₄-C₁₂ saturated or unsaturated heteroaliphatic comprising at least one O or S atom. In some embodiments, at least one instance of R⁵ is independently optionally substituted C₁₀-C₂₀ saturated or unsaturated heteroaliphatic. In some embodiments, at least one instance of R⁵ is independently optionally substituted C₁₀-C₂₀ saturated or unsaturated heteroaliphatic comprising at least one O, N, or S atom. In some embodiments, at least one instance of R⁵ is independently optionally substituted C₁₀-C₂₀ saturated or unsaturated heteroaliphatic comprising at least one O or S atom. In some embodiments, at least one instance of R⁵ is independently optionally substituted C₁₂-C₂₅ saturated or unsaturated heteroaliphatic. In some embodiments, at least one instance of R⁵ is independently optionally substituted C₁₂-C₂₅ saturated or unsaturated heteroaliphatic comprising at least one O, N, or S atom. In some embodiments, at least one instance of R⁵ is independently optionally substituted C₁₂-C₂₅ saturated or unsaturated heteroaliphatic comprising at least one O or S atom.

In some embodiments, at least one R⁵ is independently optionally substituted C₄-C₂₅ heteroalkyl or optionally substituted C₄-C₂₅ heteroalkenyl. In some embodiments, at least one R⁵ is independently optionally substituted C₄-C₁₀ heteroalkyl or optionally substituted C₄-C₁₀ heteroalkenyl. In some embodiments, at least one R⁵ is independently optionally substituted C₁₀-C₂₀ heteroalkyl or optionally substituted C₁₀-C₂₀ heteroalkenyl. In some embodiments, at least one R⁵ is independently optionally substituted C₁₂-C₂₅ heteroalkyl or optionally substituted C₁₂-C₂₅ heteroalkenyl.

In some embodiments, at least one R⁵ is independently optionally substituted C₄-C₂₅ heteroalkyl or optionally substituted C₄-C₂₅ heteroalkynyl. In some embodiments, at least one R⁵ is independently optionally substituted C₄-C₁₀ heteroalkyl or optionally substituted C₄-C₁₀ heteroalkynyl. In some embodiments, at least one R⁵ is independently optionally substituted C₁₀-C₂₀ heteroalkyl or optionally substituted C₁₀-C₂₀ heteroalkynyl. In some embodiments, at least one R⁵ is independently optionally substituted C₁₂-C₂₅ heteroalkyl or optionally substituted C₁₂-C₂₅ heteroalkynyl.

In some embodiments, at least one R⁵ is independently optionally substituted C₄-C₂₅ heteroalkynyl or optionally substituted C₄-C₂₅ heteroalkenyl. In some embodiments, at least one R⁵ is independently optionally substituted C₄-C₁₀ heteroalkynyl or optionally substituted C₄-C₁₀ heteroalkenyl. In some embodiments, at least one R⁵ is independently optionally substituted C₁₀-C₂₀ heteroalkynyl or optionally substituted C₁₀-C₂₀ heteroalkenyl. In some embodiments, at least one R⁵ is independently optionally substituted C₁₂-C₂₅ heteroalkynyl or optionally substituted C₁₂-C₂₅ heteroalkenyl.

In some embodiments, at least one R⁵ is independently branched C₄-C₂₅ heteroalkyl. In some embodiments, R⁵ is independently branched C₄-C₁₂ heteroalkyl. In some embodiments, at least one R⁵ is independently branched C₁₀-C₂₀ heteroalkyl. In some embodiments, at least one R⁵ is independently branched C₁₂-C₂₅ heteroalkyl. In some embodiments, at least one R⁵ is independently unbranched C₄-C₂₅ heteroalkyl. In some embodiments, at least one R⁵ is independently unbranched C₄-C₁₂ heteroalkyl. In some embodiments, at least one R⁵ is independently unbranched C₁₀-C₂₀ heteroalkyl. In some embodiments, at least one R⁵ is independently unbranched C₁₂-C₂₅ heteroalkyl.

In some embodiments, at least one R⁵ is independently branched C₄-C₂₅ heteroalkenyl. In some embodiments, at least one R⁵ is independently branched C₄-C₁₂ heteroalkenyl. In some embodiments, at least one R⁵ is independently branched C₁₀-C₂₀ heteroalkenyl. In some embodiments, at least one R⁵ is independently branched C₁₂-C₂₅ heteroalkenyl. In some embodiments, at least one R⁵ is independently unbranched C₄-C₂₅ heteroalkenyl. In some embodiments, at least one R⁵ is independently unbranched C₄-C₁₂ heteroalkenyl. In some embodiments, at least one R⁵ is independently unbranched C₁₀-C₂₀ heteroalkenyl. In some embodiments, at least one R⁵ is independently unbranched C₁₂-C₂₅ heteroalkenyl.

In some embodiments, at least one R⁵ is independently branched C₄-C₂₅ heteroalkynyl. In some embodiments, at least one R⁵ is independently branched C₄-C₁₂ heteroalkynyl. In some embodiments, at least one R⁵ is independently branched C₁₀-C₂₀ heteroalkynyl. In some embodiments, at least one R⁵ is independently branched C₁₂-C₂₅ heteroalkynyl. In some embodiments, at least one R⁵ is independently unbranched C₄-C₂₅ heteroalkynyl. In some embodiments, at least one R⁵ is independently unbranched C₄-C₁₂ heteroalkynyl. In some embodiments, at least one R⁵ is independently unbranched C₁₀-C₂₀ heteroalkynyl. In some embodiments, at least one R⁵ is independently unbranched C₁₂-C₂₅ heteroalkynyl.

In some embodiments, at least one R⁵ is independently selected from the group consisting of:

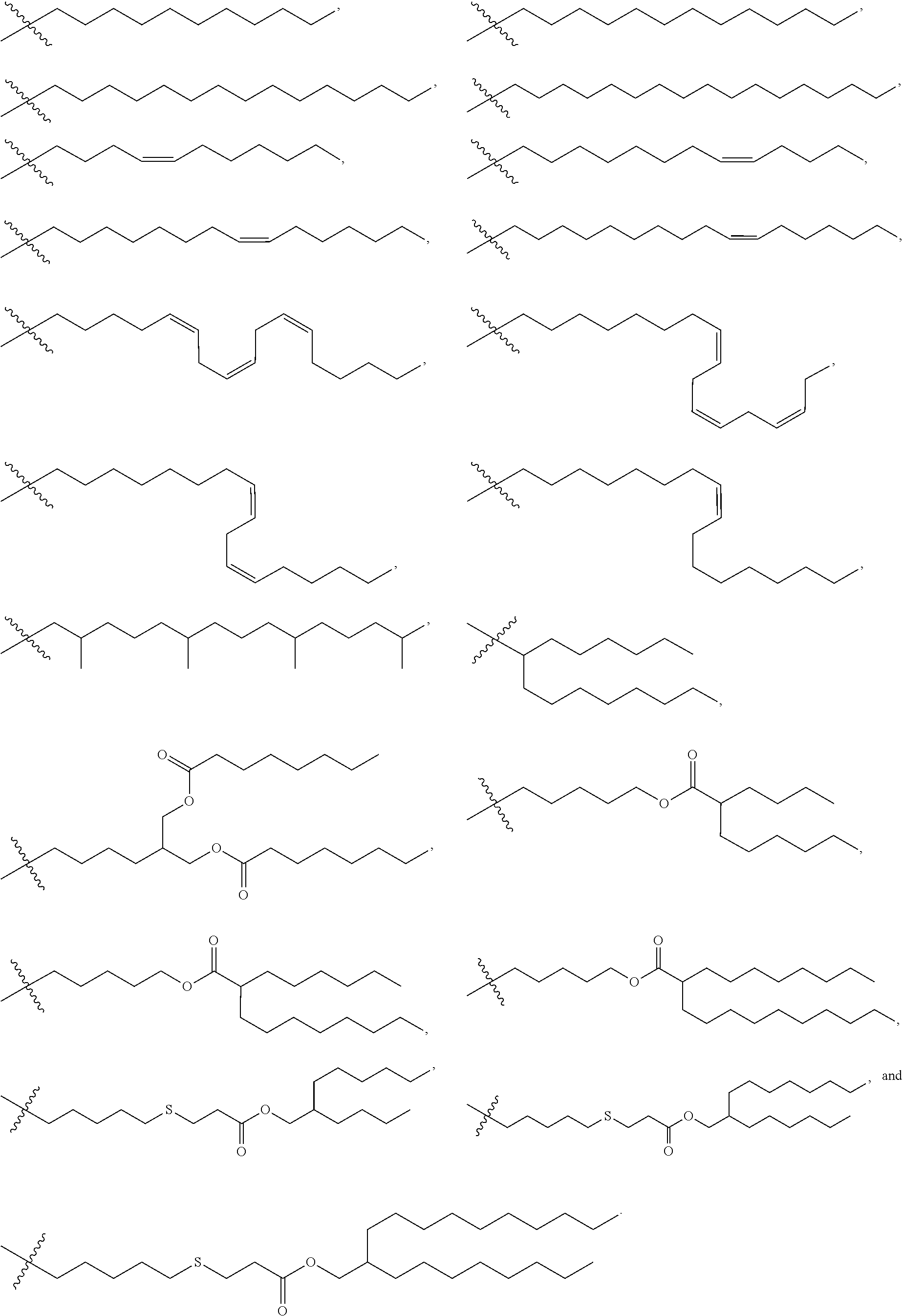

In some embodiments, at least one R⁵ is independently selected from the group consisting of:

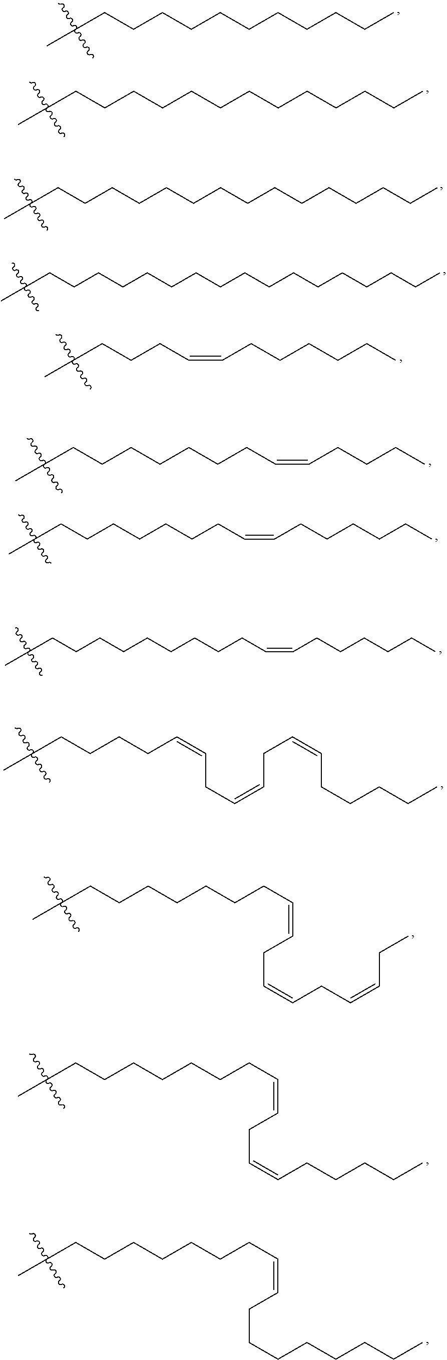

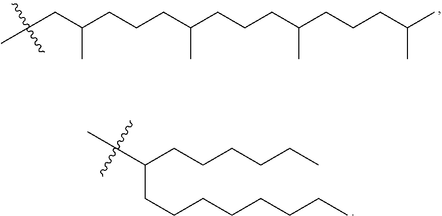 [SMILES: *CC(C)CCCC(C)CCCC(C)CCCC(C)C ; "," ; *C(CCCCCC)CCCCCCCC ; "."]

In some embodiments, at least one R⁵ is independently selected from the group consisting of:

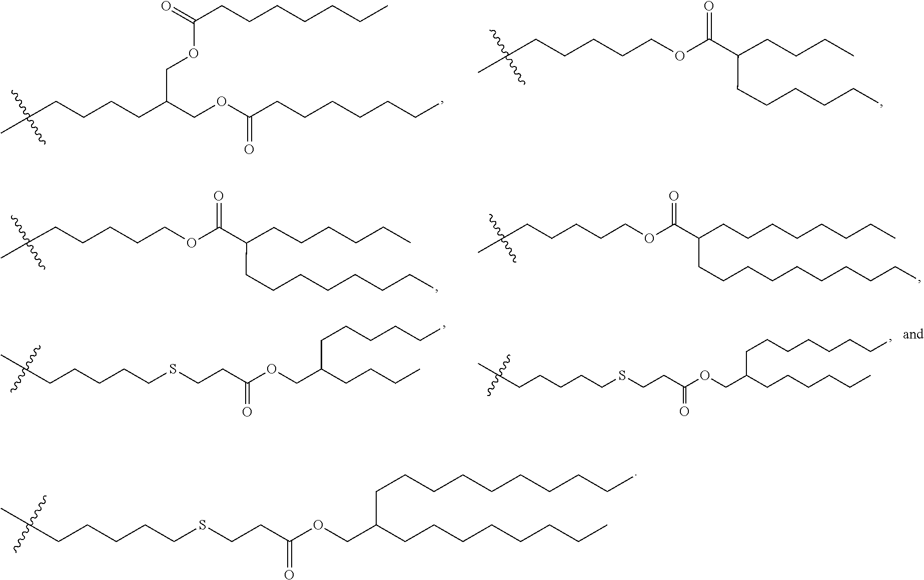

In some embodiments, at least one R⁵ is

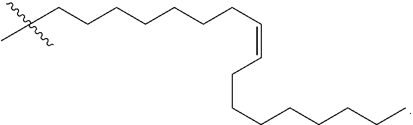 [SMILES: *CCCCCCC/C=C\CCCCCCCC]

In some embodiments, at least one R⁵ is selected from the group consisting of:

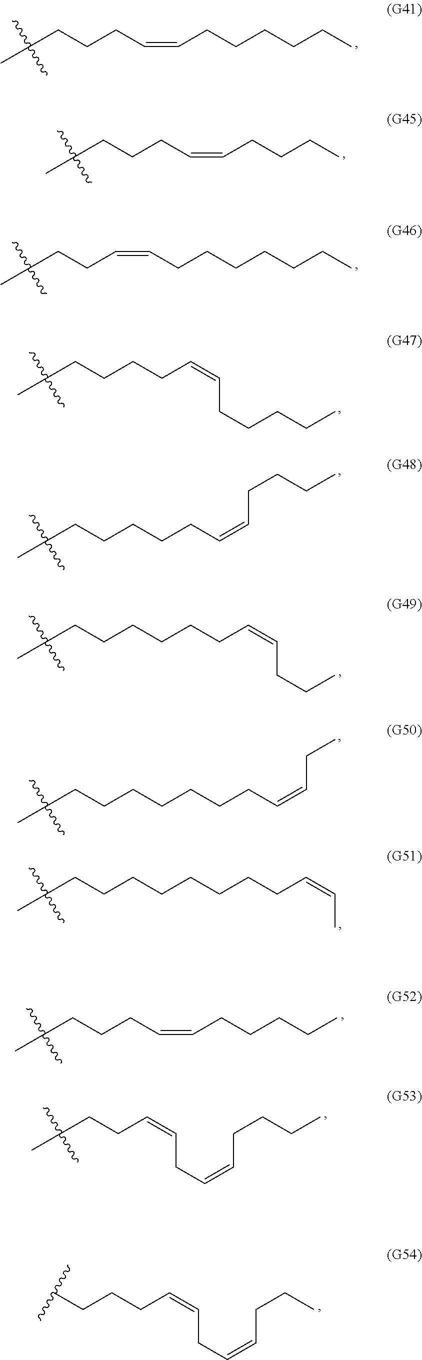

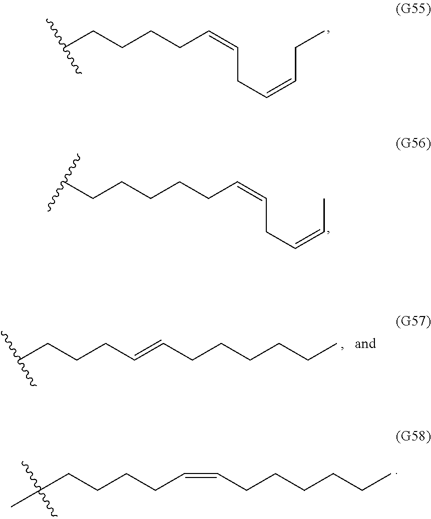

In some embodiments, at least one R⁵ is independently selected from the group consisting of:

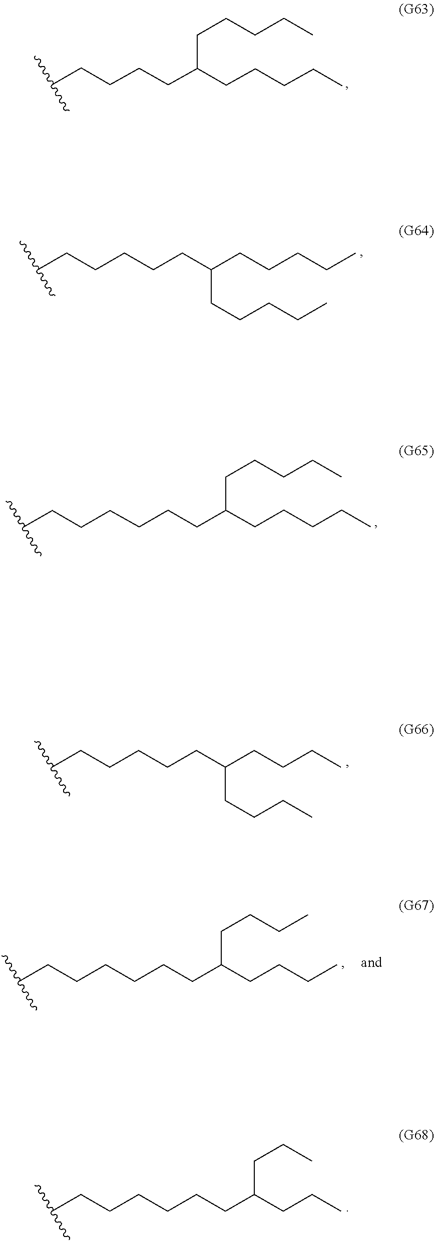 [SMILES: *CCCCC(CCCCC)CCCCC ; *CCCCCC(CCCCC)CCCCC ; *CCCCCCC(CCCCC)CCCCC ; *CCCCCC(CCCC)CCCC ; *CCCCCCC(CCCC)CCCC ; *CCCCCCC(CCC)CCC]

In a particular embodiment, at least one R⁵ is G41. In a particular embodiment, at least one R⁵ is G45. In a particular embodiment, at least one R⁵ is G46. In a particular embodiment, at least one R⁵ is G47. In a particular embodiment, at least one R⁵ is G48. In a particular embodiment, at least one R⁵ is G49. In a particular embodiment, at least one R⁵ is G50. In a particular embodiment, at least one R⁵ is G51. In a particular embodiment, at least one R⁵ is G52. In a particular embodiment, at least one R⁵ is G53. In a particular embodiment, at least one R⁵ is G54. In a particular embodiment, at least one R⁵ is G55. In a particular embodiment, at least one R⁵ is G56. In a particular embodiment, at least one R⁵ is G57. In a particular embodiment, at least one R⁵ is G58. In a particular embodiment, at least one R⁵ is G63. In a particular embodiment, at least one R⁵ is G64. In a particular embodiment, at least one R⁵ is G65. In a particular embodiment, at least one R⁵ is G66. In a particular embodiment, at least one R⁵ is G67. In a particular embodiment, at least one R⁵ is G68.

As defined herein, each x independently is an integer from 1 to 10, inclusive. In some embodiments, each x independently is an integer from 1 to 9, inclusive. In some embodiments, each x independently is an integer from 1 to 8, inclusive. In some embodiments, each x independently is an integer from 1 to 7, inclusive. In some embodiments, each x independently is an integer from 1 to 6, inclusive. In some embodiments, each x independently is an integer from 1 to 5, inclusive. In some embodiments, each x independently is an integer from 1 to 4, inclusive. In some embodiments, each x independently is an integer from 1 to 3, inclusive.

In some embodiments, each x is the same. In some embodiments, at least two instances of x are the same. In some embodiments, at least three instances of x are the same. In some embodiments, four instances of x are the same.

In some embodiments, x is 1. In some embodiments, x is 2. In some embodiments, x is 3. In some embodiments, x is 4. In some embodiments, x is 5. In some embodiments, x is 6. In some embodiments, x is 7. In some embodiments, x is 8. In some embodiments, x is 9. In some embodiments, x is 10.

In some embodiments, each x is 1. In some embodiments, each x is 2. In some embodiments, each x is 3. In some embodiments, each x is 4. In some embodiments, each x is 5. In some embodiments, each x is 6. In some embodiments, each x is 7. In some embodiments, each x is 8. In some embodiments, each x is 9. In some embodiments, each x is 10.

In some embodiments, the compound of Formula (I) is selected from:

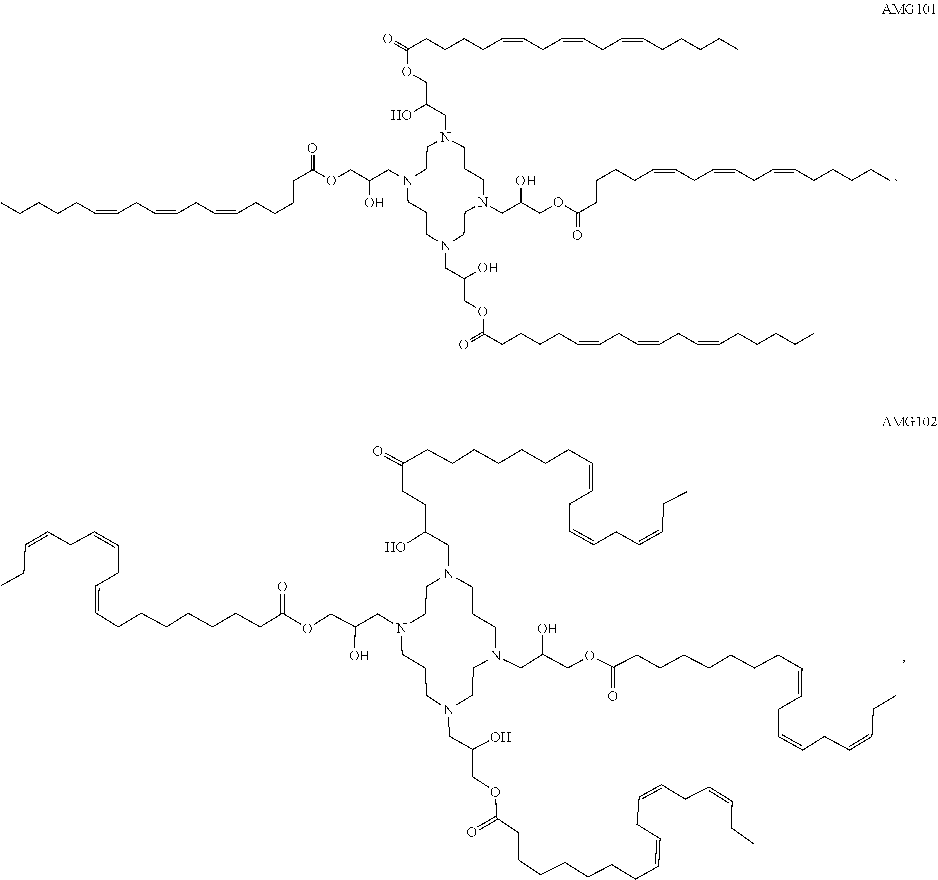

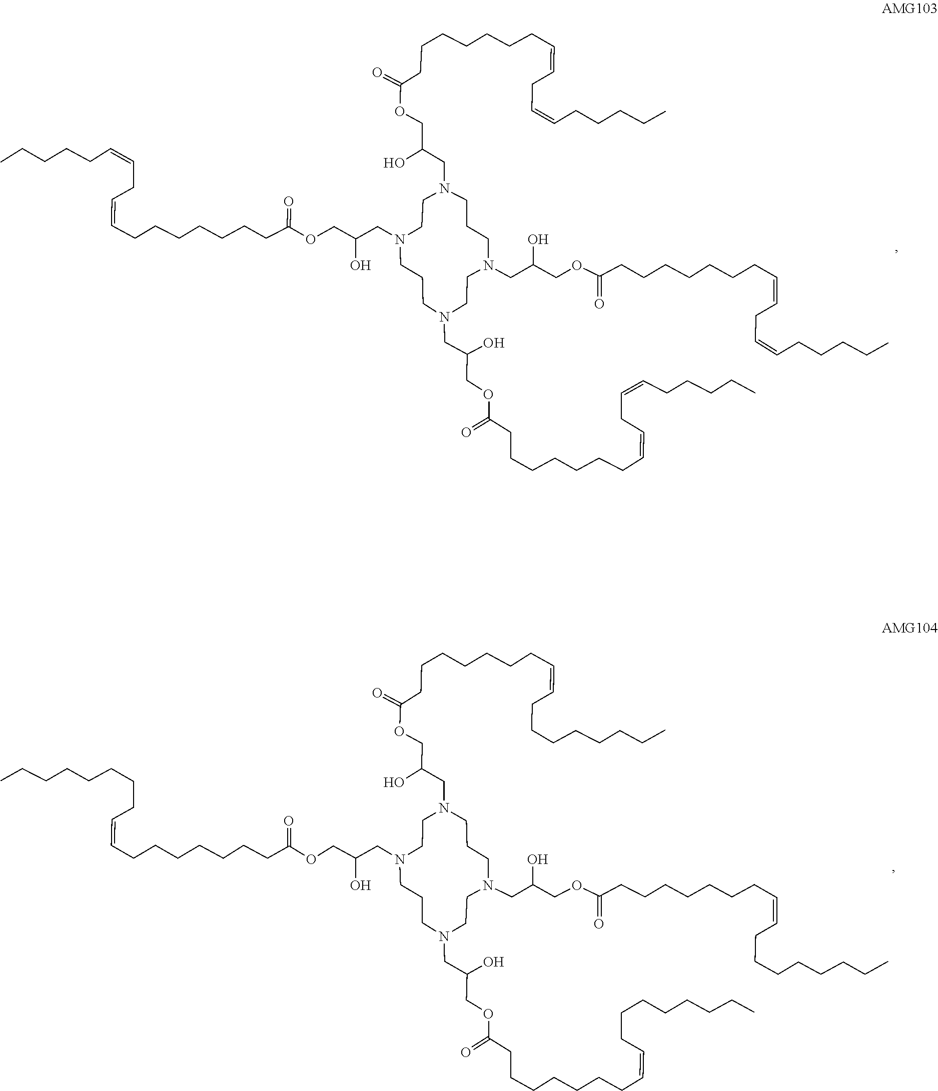

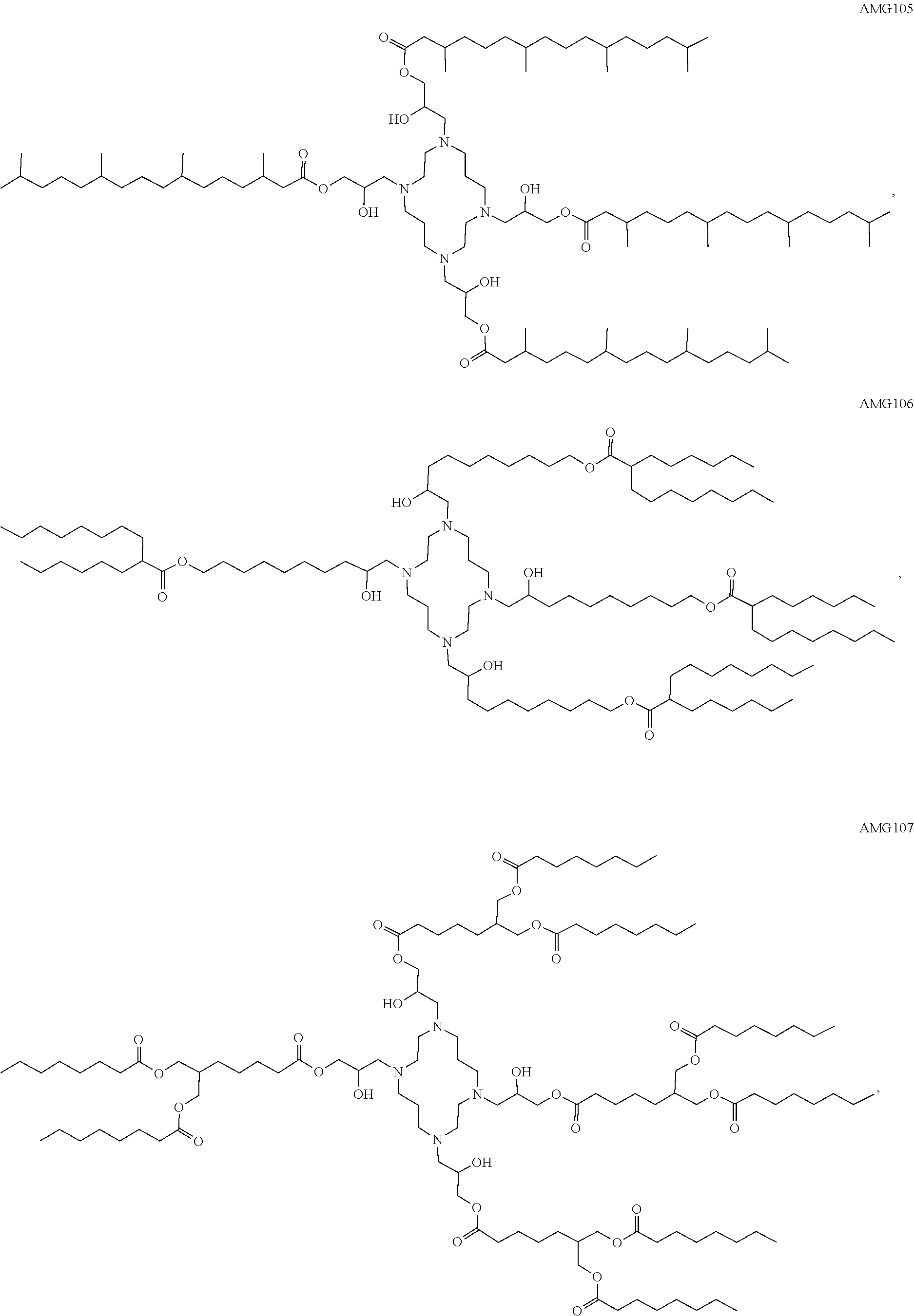

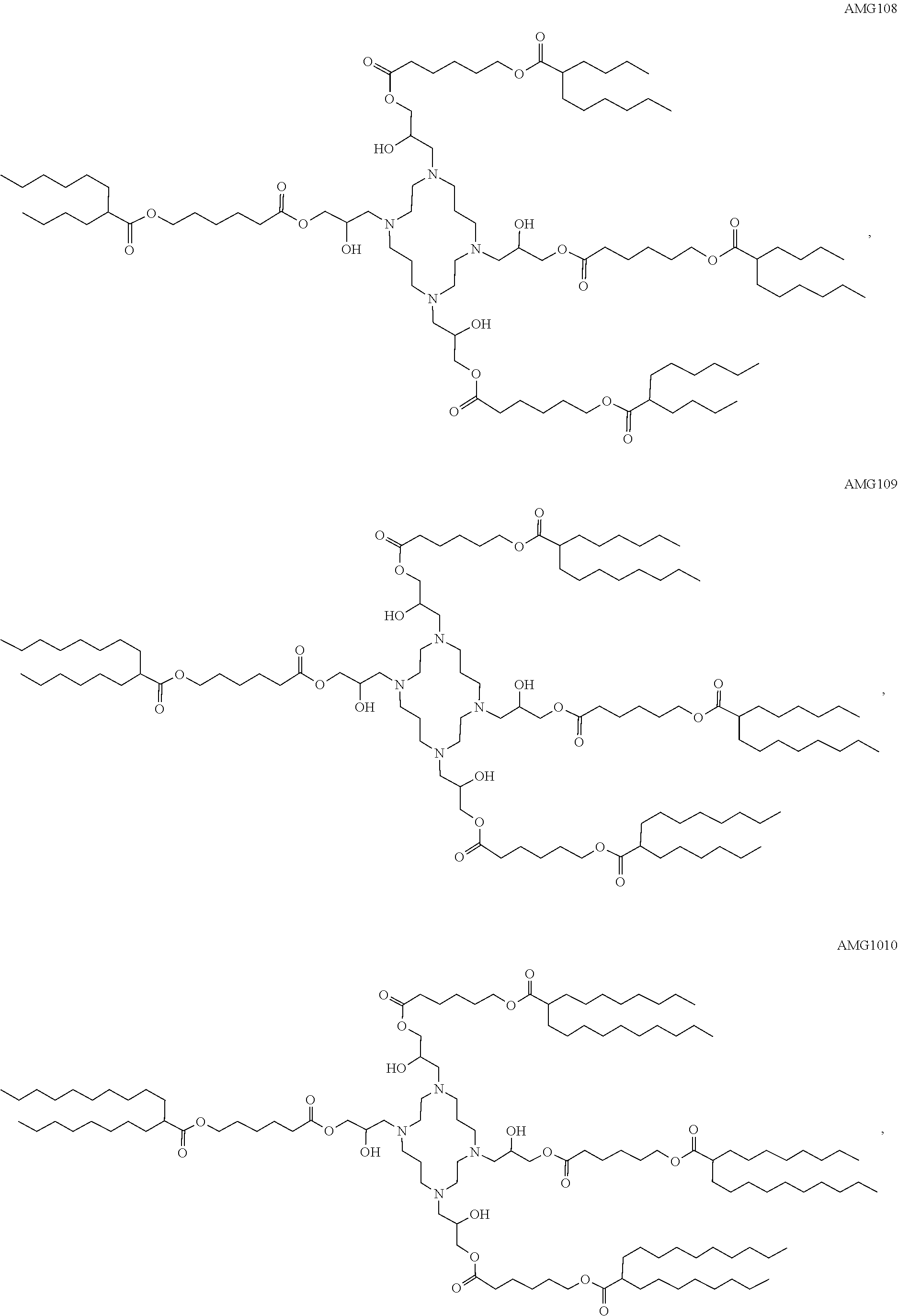

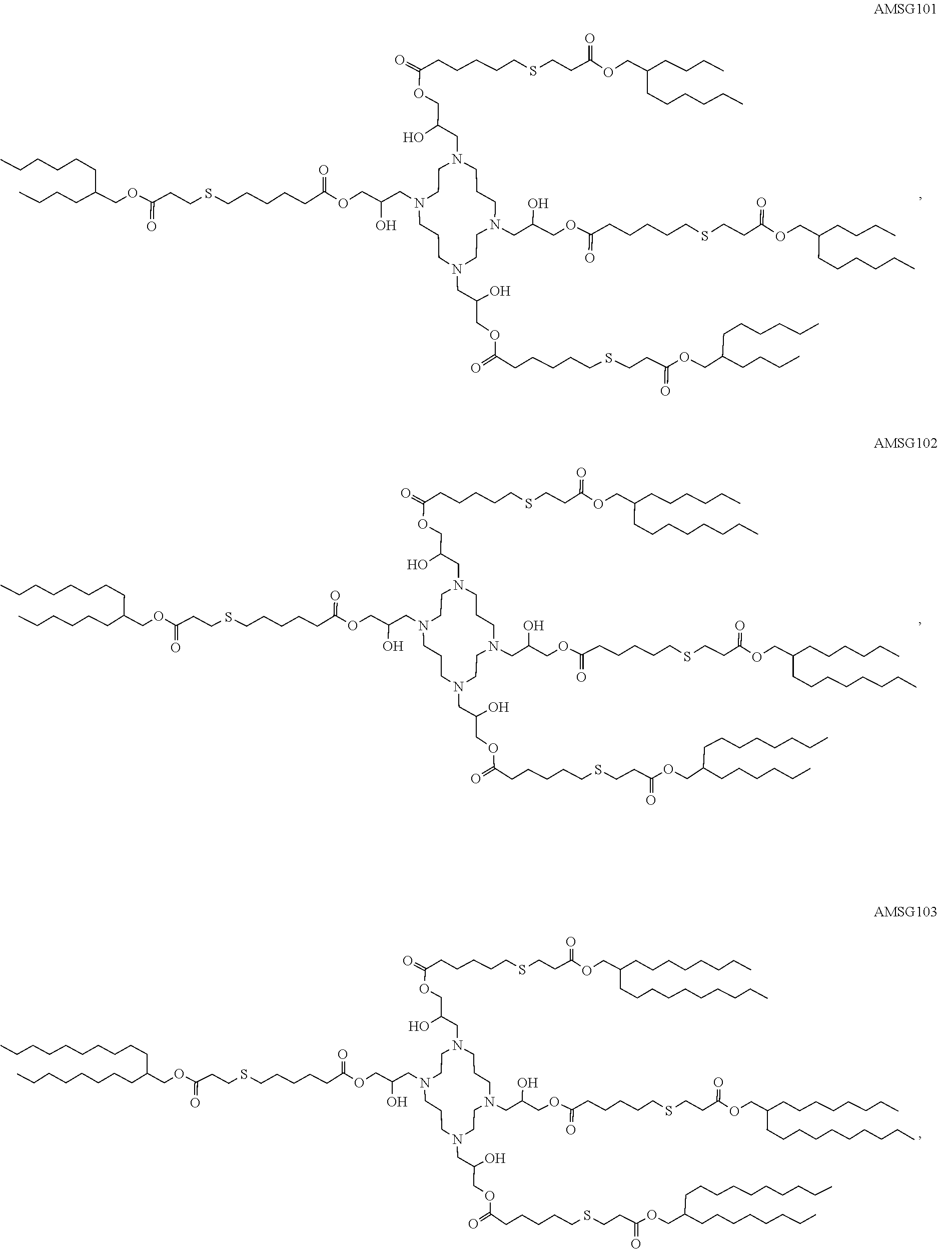

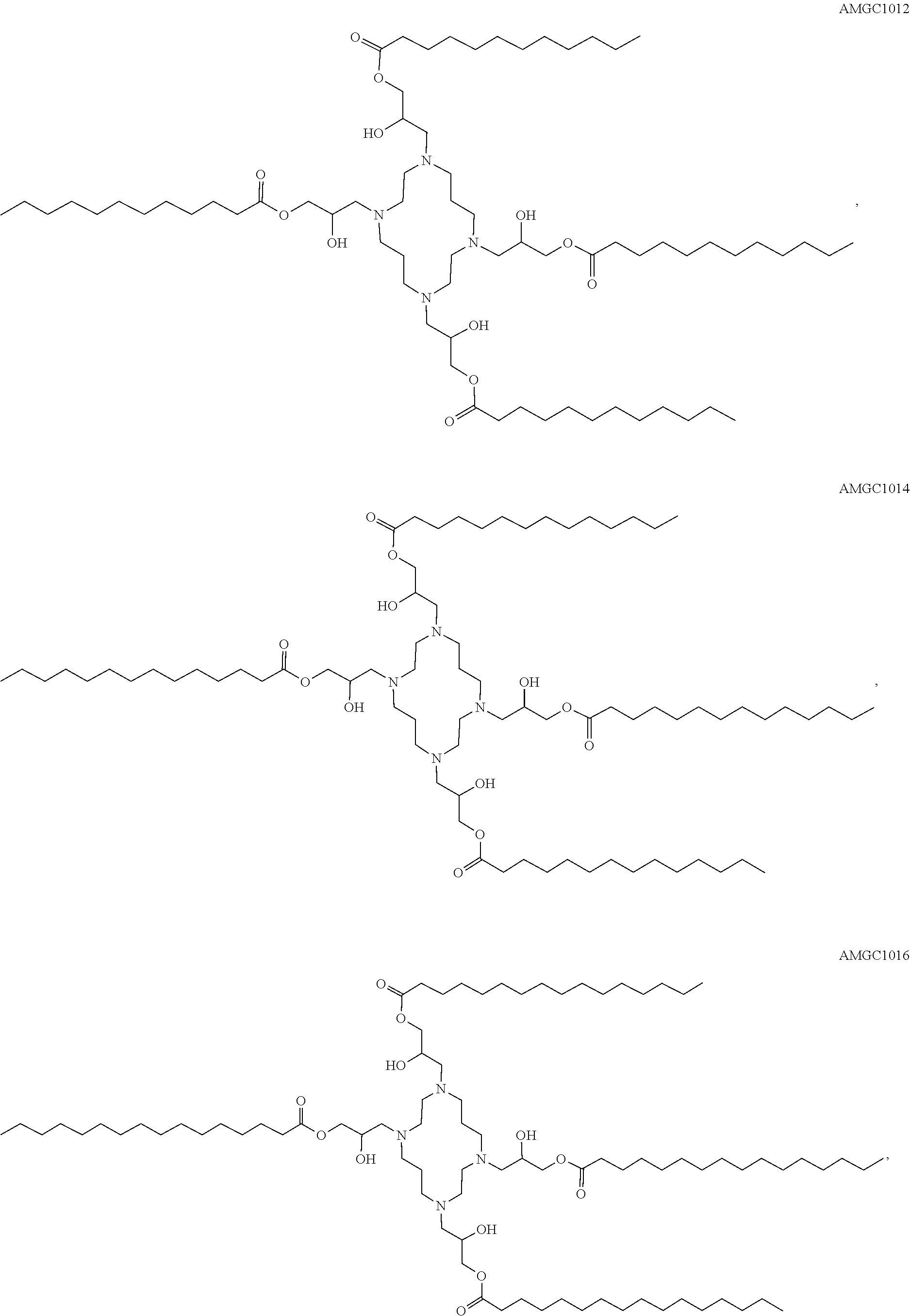

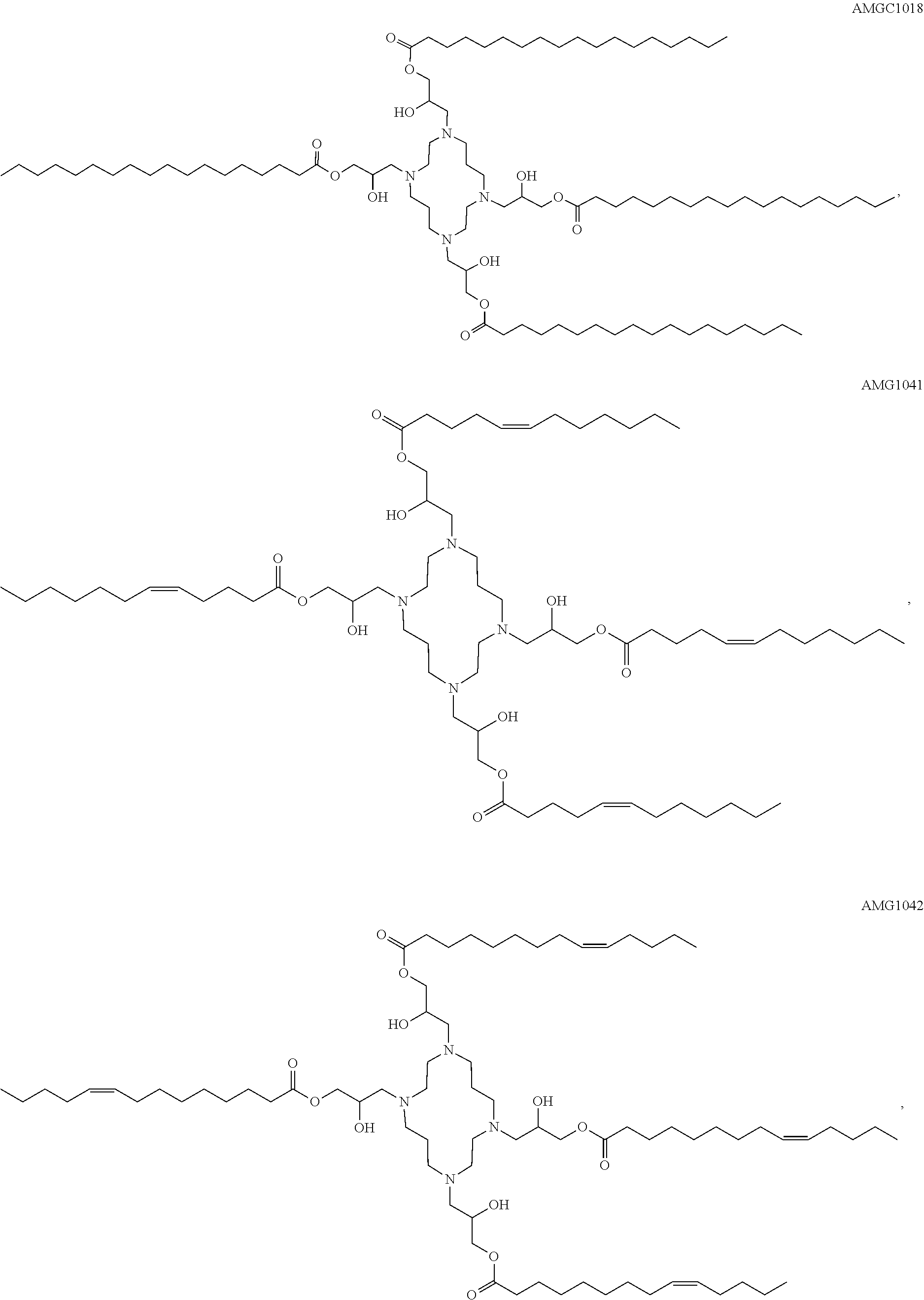

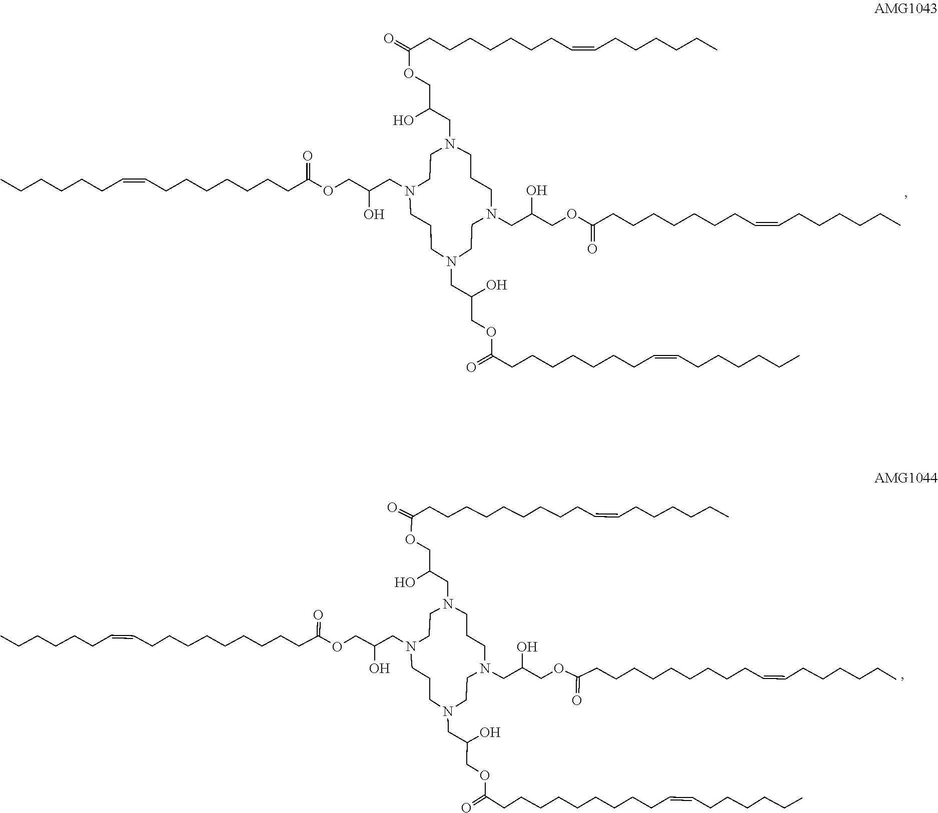

and pharmaceutically acceptable salts thereof.

In some embodiments, the compound of Formula (I) is:

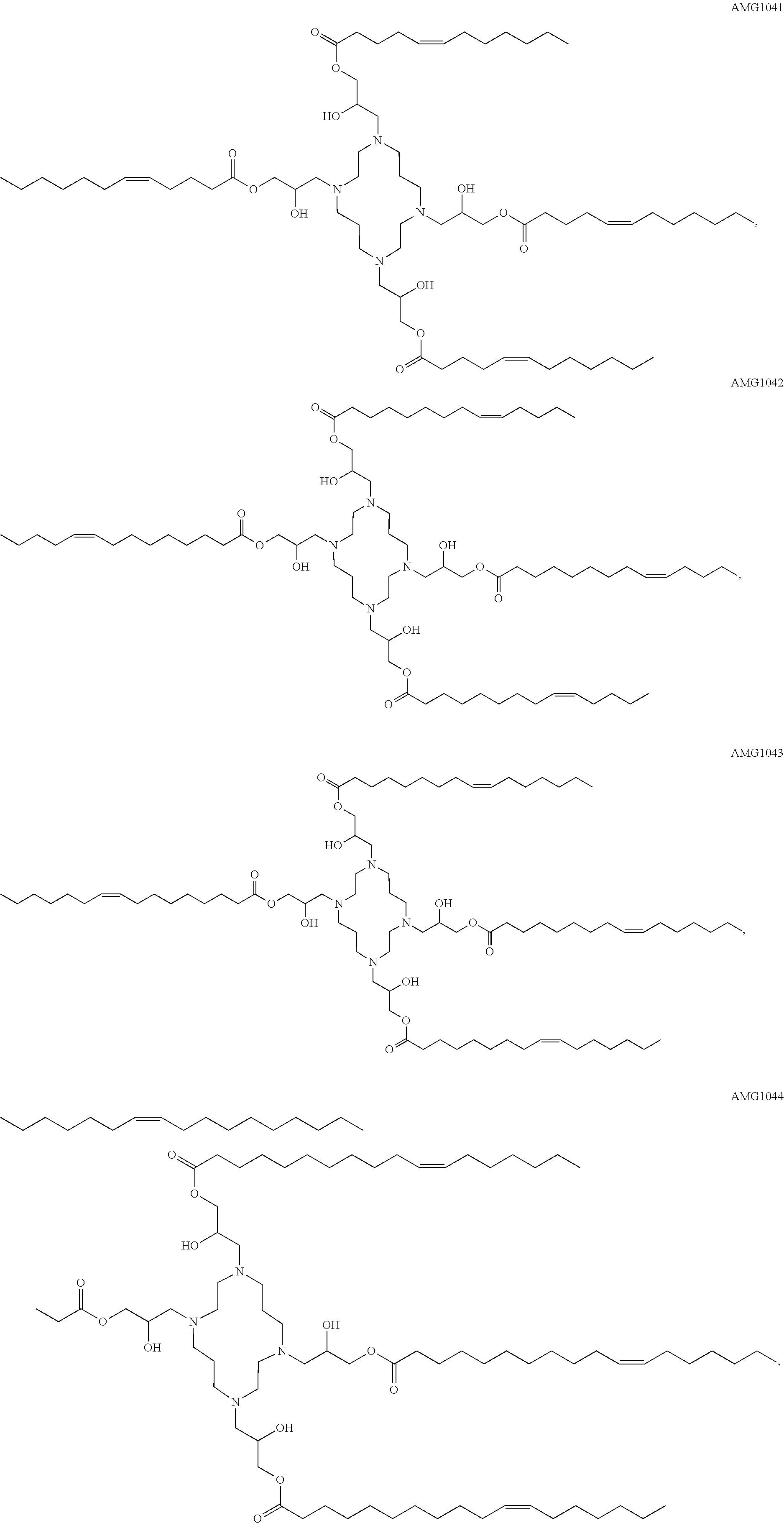

or a pharmaceutically acceptable salt thereof.

In some embodiments, the compound of Formula (I) is:

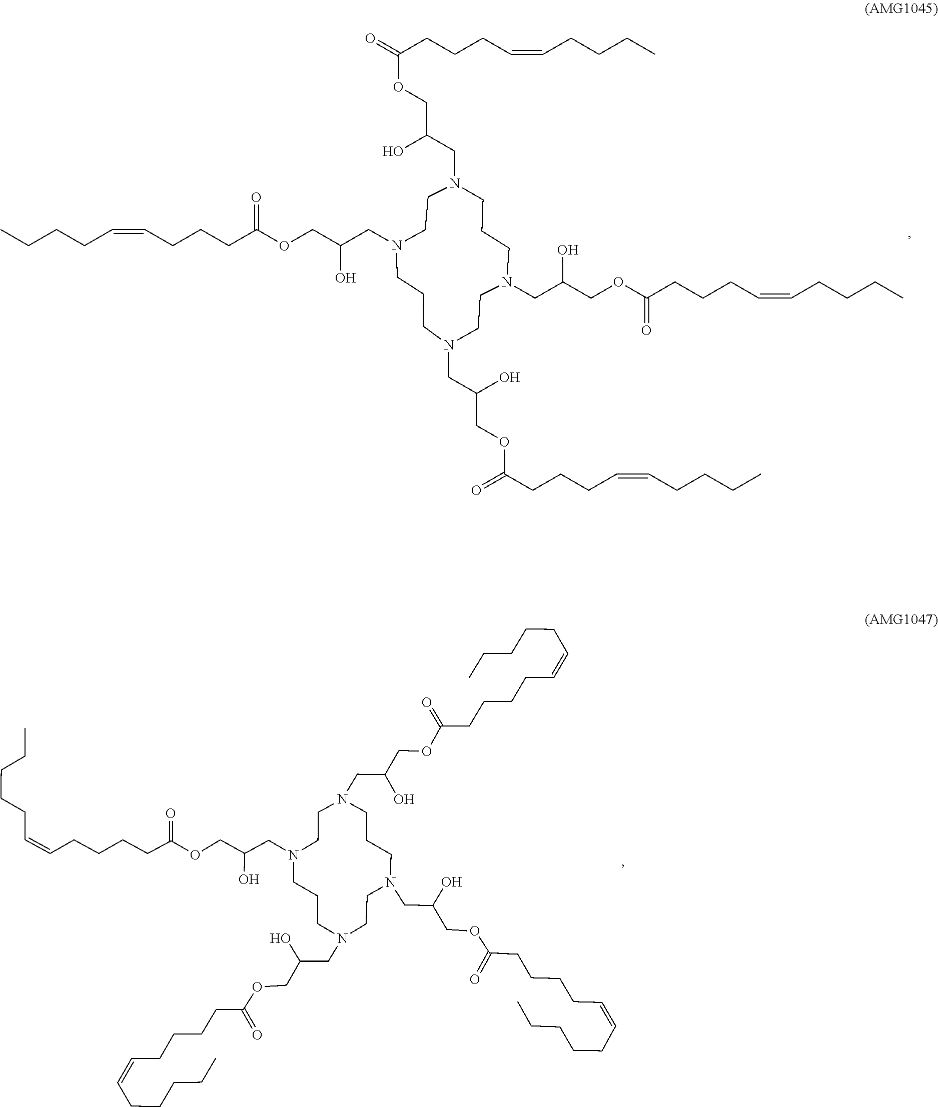 [SMILES: CCCC/C=C\CCCC(=O)OCC(O)CN1CCCN(CC(O)COC(=O)CCC/C=C\CCCC)CCN(CC(O)COC(=O)CCC/C=C\CCCC)CCCN(CC(O)COC(=O)CCC/C=C\CCCC)CC1 ; CCCCC/C=C\CCCCC(=O)OCC(O)CN1CCCN(CC(O)COC(=O)CCCC/C=C\CCCCC)CCN(CC(O)COC(=O)CCCC/C=C\CCCCC)CCCN(CC(O)COC(=O)CCCC/C=C\CCCCC)CC1]

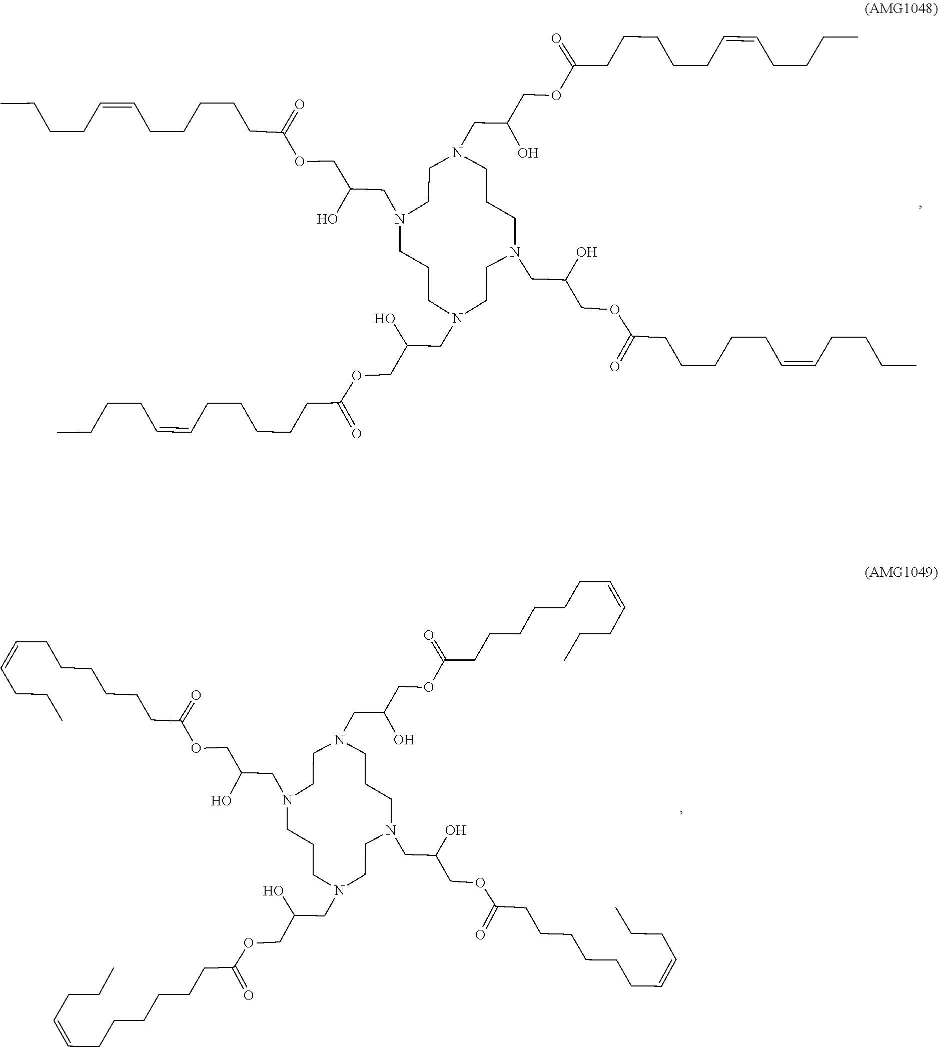 [SMILES: CCCC/C=C\CCCCCC(=O)OCC(O)CN1CCCN(CC(O)COC(=O)CCCCC/C=C\CCCC)CCN(CC(O)COC(=O)CCCCC/C=C\CCCC)CCCN(CC(O)COC(=O)CCCCC/C=C\CCCC)CC1 ; CCC/C=C\CCCCCCC(=O)OCC(O)CN1CCCN(CC(O)COC(=O)CCCCCC/C=C\CCC)CCN(CC(O)COC(=O)CCCCCC/C=C\CCC)CCCN(CC(O)COC(=O)CCCCCC/C=C\CCC)CC1]

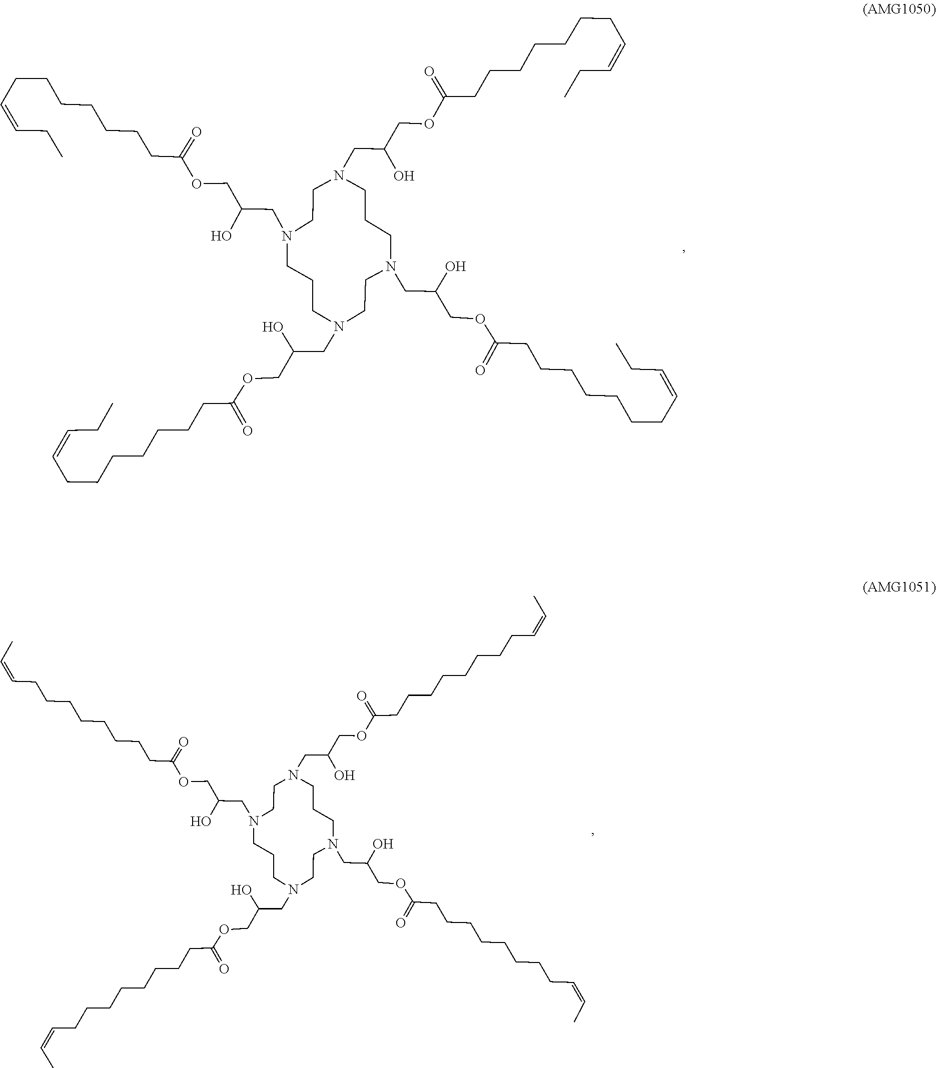 [SMILES: CC/C=C\CCCCCCCC(=O)OCC(O)CN1CCCN(CC(O)COC(=O)CCCCCCC/C=C\CC)CCN(CC(O)COC(=O)CCCCCCC/C=C\CC)CCCN(CC(O)COC(=O)CCCCCCC/C=C\CC)CC1 ; C/C=C\CCCCCCCCC(=O)OCC(O)CN1CCCN(CC(O)COC(=O)CCCCCCCC/C=C\C)CCN(CC(O)COC(=O)CCCCCCCC/C=C\C)CCCN(CC(O)COC(=O)CCCCCCCC/C=C\C)CC1]

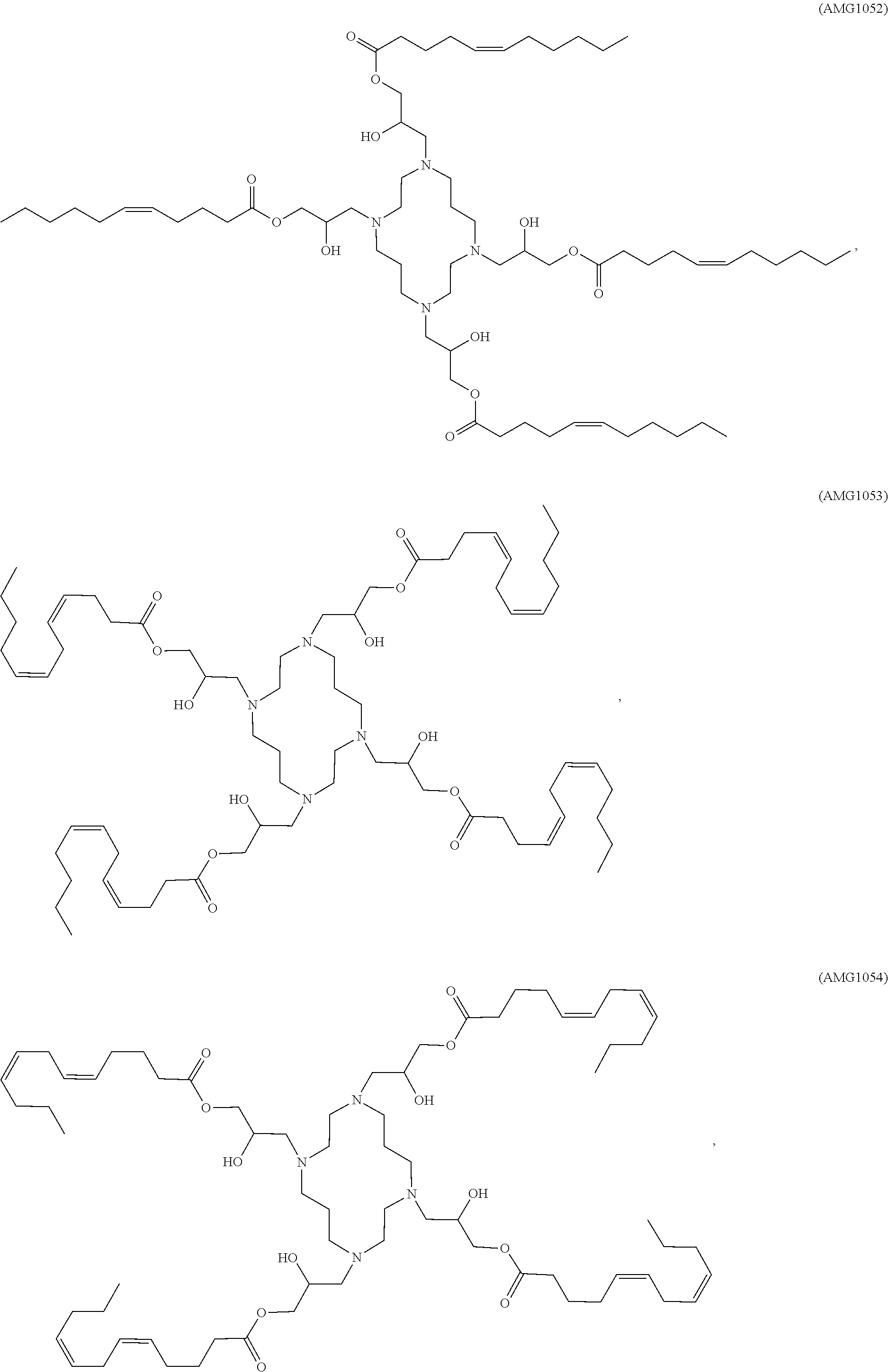 [SMILES: CCCCC/C=C\CCCC(=O)OCC(O)CN1CCCN(CC(O)COC(=O)CCC/C=C\CCCCC)CCN(CC(O)COC(=O)CCC/C=C\CCCCC)CCCN(CC(O)COC(=O)CCC/C=C\CCCCC)CC1 ; CCCC/C=C\C/C=C\CCC(=O)OCC(O)CN1CCCN(CC(O)COC(=O)CC/C=C\C/C=C\CCCC)CCN(CC(O)COC(=O)CC/C=C\C/C=C\CCCC)CCCN(CC(O)COC(=O)CC/C=C\C/C=C\CCCC)CC1 ; CCC/C=C\C/C=C\CCCC(=O)OCC(O)CN1CCCN(CC(O)COC(=O)CCC/C=C\C/C=C\CCC)CCN(CC(O)COC(=O)CCC/C=C\C/C=C\CCC)CCCN(CC(O)COC(=O)CCC/C=C\C/C=C\CCC)CC1]

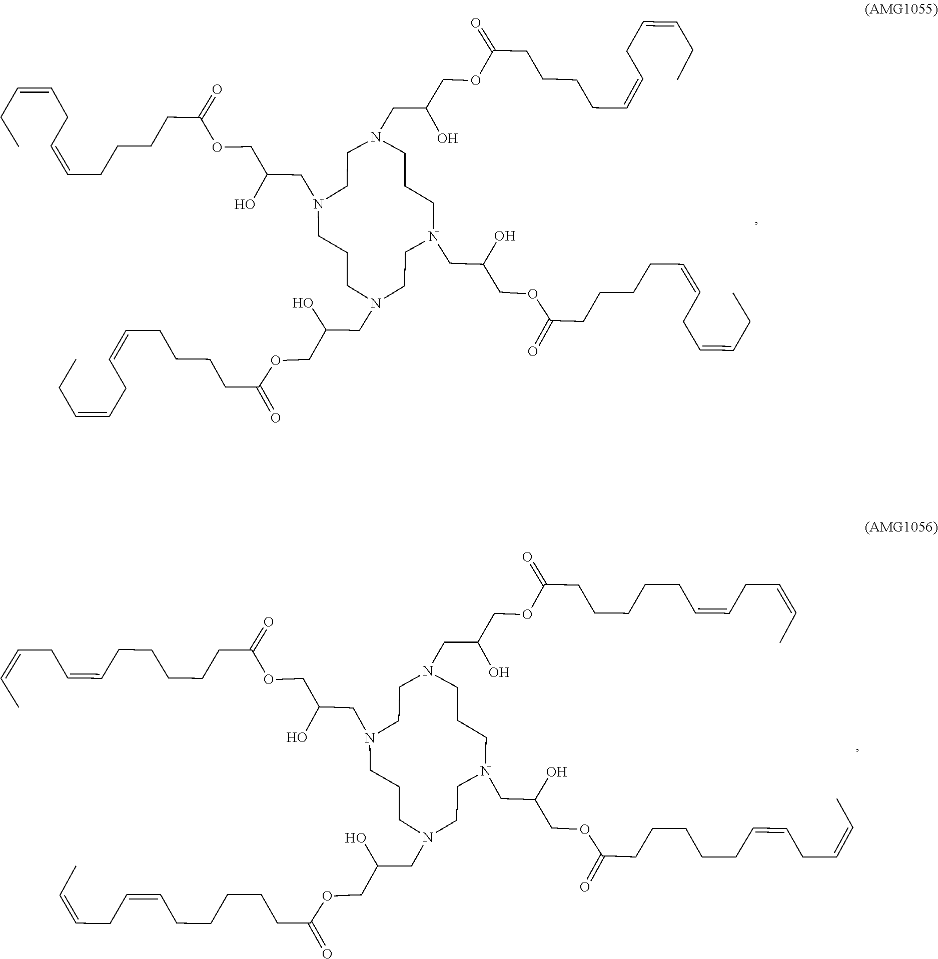 [SMILES: CC/C=C\C/C=C\CCCCC(=O)OCC(O)CN1CCCN(CC(O)COC(=O)CCCC/C=C\C/C=C\CC)CCN(CC(O)COC(=O)CCCC/C=C\C/C=C\CC)CCCN(CC(O)COC(=O)CCCC/C=C\C/C=C\CC)CC1 ; C/C=C\C/C=C\CCCCCC(=O)OCC(O)CN1CCCN(CC(O)COC(=O)CCCCC/C=C\C/C=C\C)CCN(CC(O)COC(=O)CCCCC/C=C\C/C=C\C)CCCN(CC(O)COC(=O)CCCCC/C=C\C/C=C\C)CC1]

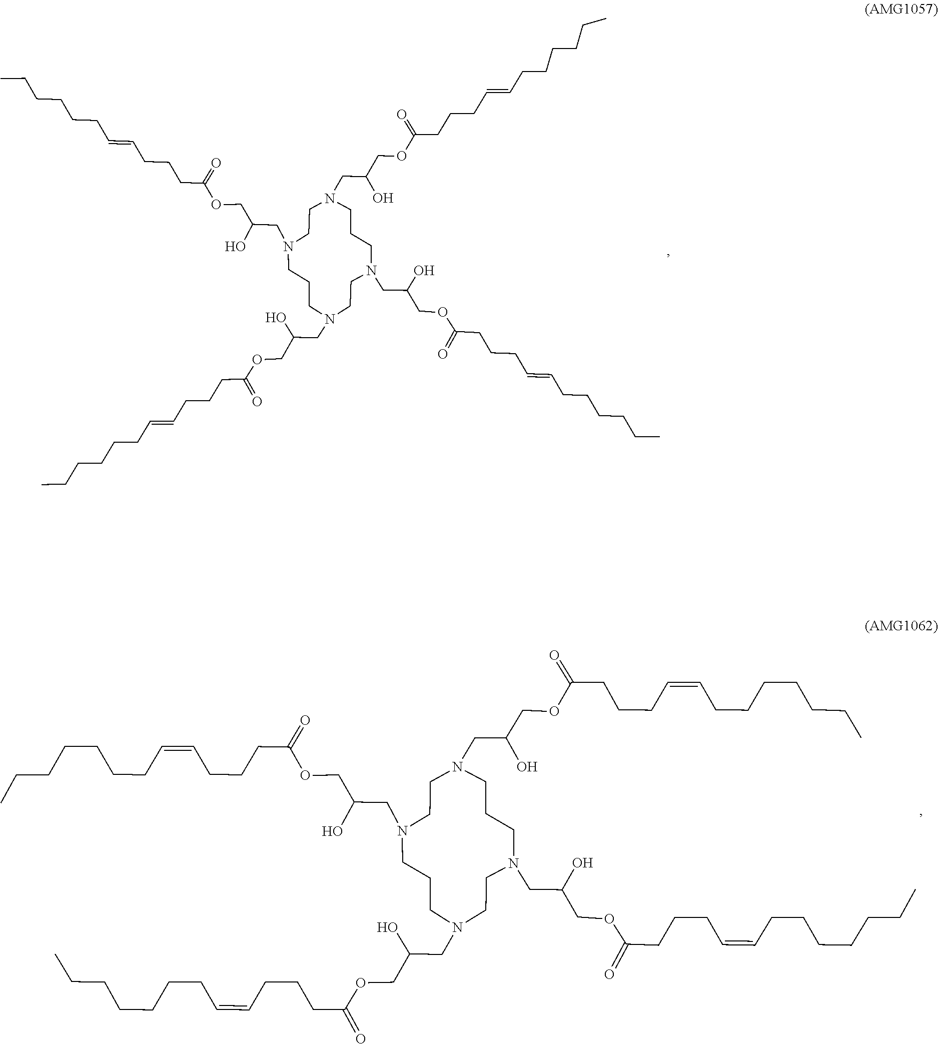

or a pharmaceutically acceptable salt thereof.

In some embodiments, the compound of Formula (I) is:

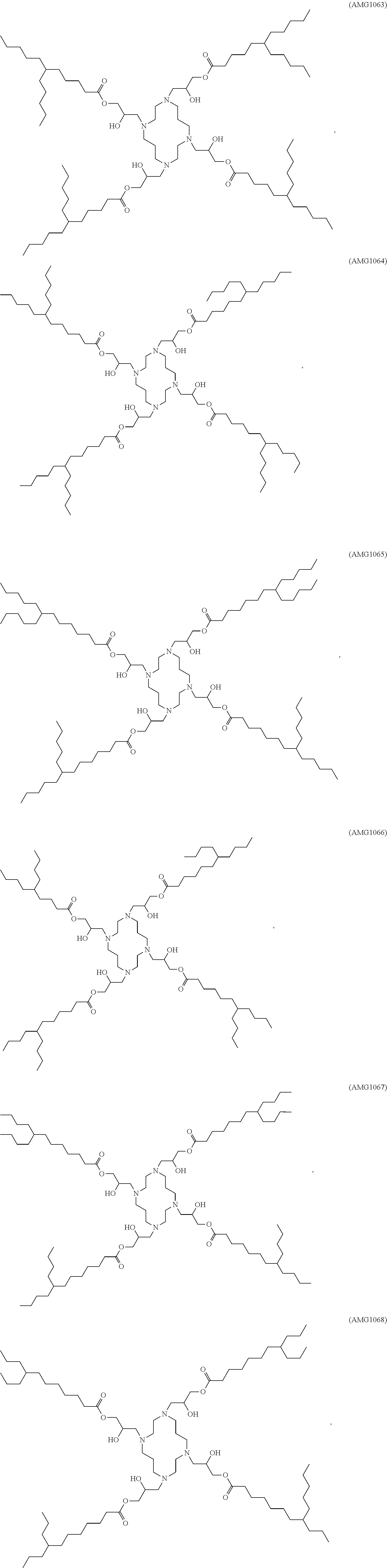

or a pharmaceutically acceptable salt thereof.

In some embodiments, the compound of Formula (I) is:

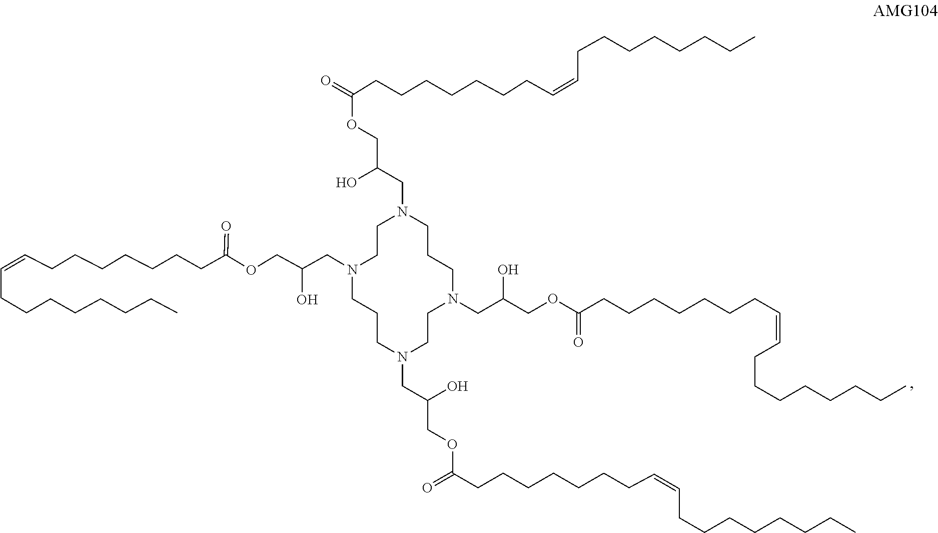 [SMILES: CCCCCCCC/C=C\CCCCCCCC(=O)OCC(O)CN1CCCN(CC(O)COC(=O)CCCCCCC/C=C\CCCCCCCC)CCN(CC(O)COC(=O)CCCCCCC/C=C\CCCCCCCC)CCCN(CC(O)COC(=O)CCCCCCC/C=C\CCCCCCCC)CC1]

or a pharmaceutically acceptable salt thereof. In some embodiments, the compound of Formula (I) is AMG1041, or a pharmaceutically acceptable salt thereof. In some embodiments, the compound of Formula (I) is AMG1043, or a pharmaceutically acceptable salt thereof. In some embodiments, the compound of Formula (I) is AMG1044, or a pharmaceutically acceptable salt thereof. In some embodiments, the compound of Formula (I) is AMG1045, or a pharmaceutically acceptable salt thereof. In some embodiments, the compound of Formula (I) is AMG1046, or a pharmaceutically acceptable salt thereof. In some embodiments, the compound of Formula (I) is AMG1047, or a pharmaceutically acceptable salt thereof. In some embodiments, the compound of Formula (I) is AMG1048, or a pharmaceutically acceptable salt thereof. In some embodiments, the compound of Formula (I) is AMG1049, or a pharmaceutically acceptable salt thereof. In some embodiments, the compound of Formula (I) is AMG1050, or a pharmaceutically acceptable salt thereof. In some embodiments, the compound of Formula (I) is AMG1051, or a pharmaceutically acceptable salt thereof. In some embodiments, the compound of Formula (I) is AMG1052, or a pharmaceutically acceptable salt thereof. In some embodiments, the compound of Formula (I) is AMG1053, or a pharmaceutically acceptable salt thereof. In some embodiments, the compound of Formula (I) is AMG1054, or a pharmaceutically acceptable salt thereof. In some embodiments, the compound of Formula (I) is AMG1055, or a pharmaceutically acceptable salt thereof. In some embodiments, the compound of Formula (I) is AMG1056, or a pharmaceutically acceptable salt thereof. In some embodiments, the compound of Formula (I) is AMG1057, or a pharmaceutically acceptable salt thereof. In some embodiments, the compound of Formula (I) is AMG1062, or a pharmaceutically acceptable salt thereof. In some embodiments, the compound of Formula (I) is AMG1063, or a pharmaceutically acceptable salt thereof. In some embodiments, the compound of Formula (I) is AMG1064, or a pharmaceutically acceptable salt thereof. In some embodiments, the compound of Formula (I) is AMG1065, or a pharmaceutically acceptable salt thereof. In some embodiments, the compound of Formula (I) is AMG1066, or a pharmaceutically acceptable salt thereof. In some embodiments, the compound of Formula I) is AMG1067, or a pharmaceutically acceptable salt thereof. In some embodiments, the compound of Formula (I) is AMG1068, or a pharmaceutically acceptable salt thereof.

In some embodiments, the compound of Formula (IA) is:

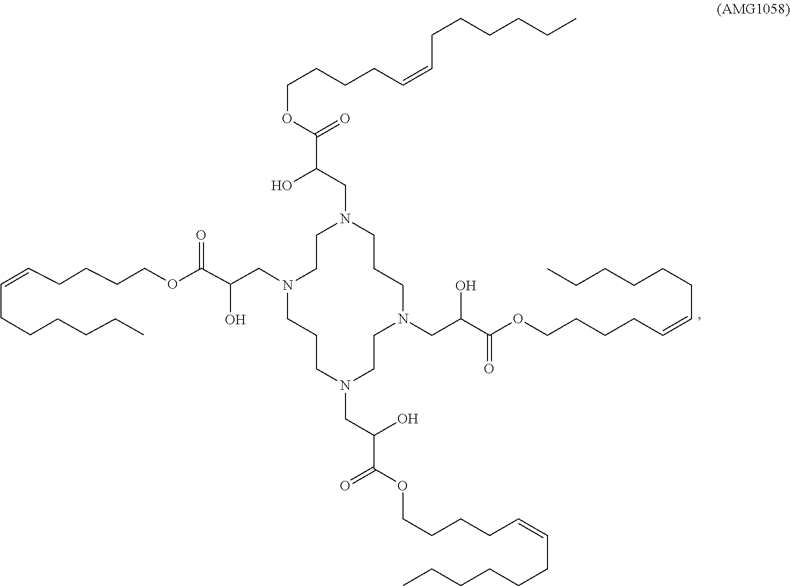 [SMILES: CCCCCC/C=C\CCCCOC(=O)C(O)CN1CCCN(CC(O)C(=O)OCCCC/C=C\CCCCCC)CCN(CC(O)C(=O)OCCCC/C=C\CCCCCC)CCCN(CC(O)C(=O)OCCCC/C=C\CCCCCC)CC1]

or a pharmaceutically acceptable salt thereof.

In some embodiments, the compound of Formula (IA) is:

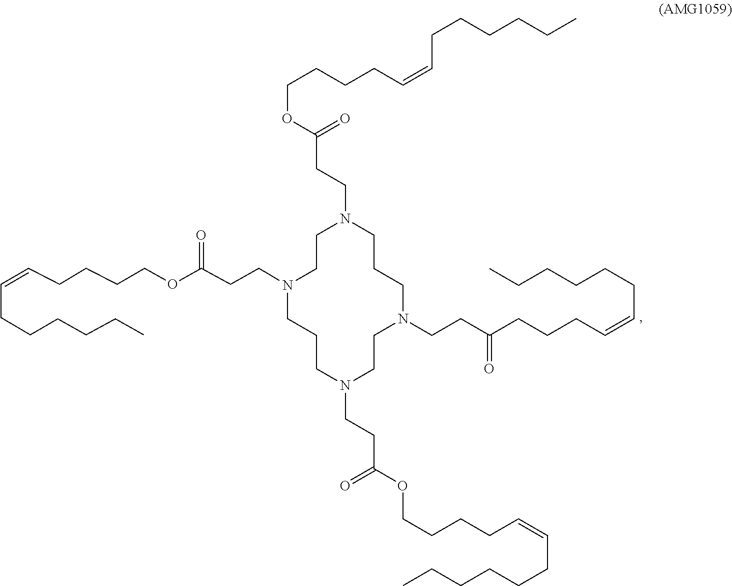 [SMILES: CCCCCC/C=C\CCCCOC(=O)CCN1CCCN(CCC(=O)OCCCC/C=C\CCCCCC)CCN(CCC(=O)OCCCC/C=C\CCCCCC)CCCN(CCC(=O)CCC/C=C\CCCCCC)CC1]

or a pharmaceutically acceptable salt thereof.

In some embodiments, the compound of Formula (IA) is:

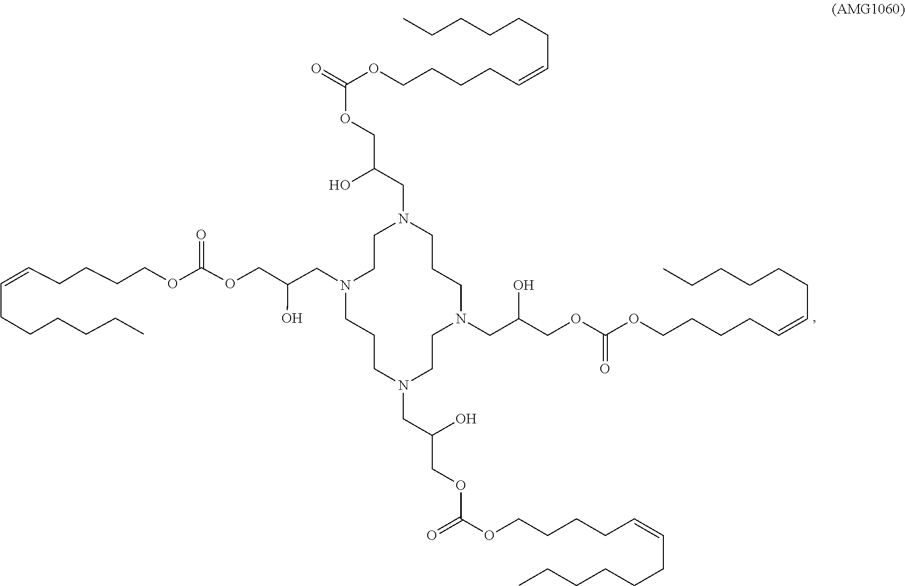 [SMILES: CCCCCC/C=C\CCCCOC(=O)OCC(O)CN1CCCN(CC(O)COC(=O)OCCCC/C=C\CCCCCC)CCN(CC(O)COC(=O)OCCCC/C=C\CCCCCC)CCCN(CC(O)COC(=O)OCCCC/C=C\CCCCCC)CC1]

or a pharmaceutically acceptable salt thereof.

In some embodiments, the compound of Formula (IA) is:

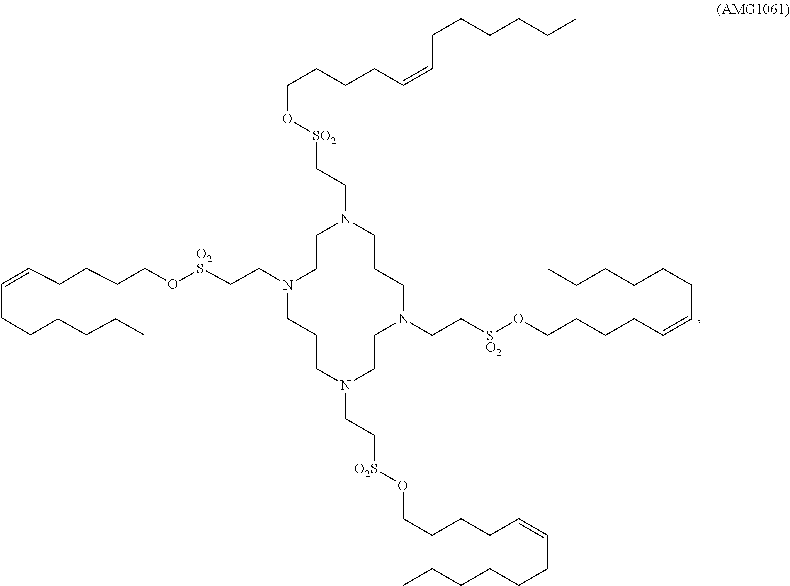 [SMILES: CCCCCC/C=C\CCCCOS(=O)(=O)CCN1CCCN(CCS(=O)(=O)OCCCC/C=C\CCCCCC)CCN(CCS(=O)(=O)OCCCC/C=C\CCCCCC)CCCN(CCS(=O)(=O)OCCCC/C=C\CCCCCC)CC1]

or a pharmaceutically acceptable salt thereof.

In some embodiments, the compound of Formula (IA) is AMG1041. In some embodiments, the compound of Formula (IA) is AMG1043. In some embodiments, the compound of Formula (IA) is AMG1044. In some embodiments, the compound of Formula (IA) is AMG1045. In some embodiments, the compound of Formula (IA) is AMG1046. In some embodiments, the compound of Formula (IA) is AMG1047. In some embodiments, the compound of Formula (IA) is AMG1048. In some embodiments, the compound of Formula (IA) is AMG1049. In some embodiments, the compound of Formula (IA) is AMG1050. In some embodiments, the compound of Formula (IA) is AMG1051. In some embodiments, the compound of Formula (IA) is AMG1052. In some embodiments, the compound of Formula (IA) is AMG1053. In some embodiments, the compound of Formula (IA) is AMG1054. In some embodiments, the compound of Formula (IA) is AMG1055. In some embodiments, the compound of Formula (IA) is AMG1056. In some embodiments, the compound of Formula (IA) is AMG1057. In some embodiments, the compound of Formula (IA) is AMG1058. In some embodiments, the compound of Formula (IA) is AMG1059. In some embodiments, the compound of Formula (IA) is AMG1060. In some embodiments, the compound of Formula (IA) is AMG1061. In some embodiments, the compound of Formula (IA) is AMG1062. In some embodiments, the compound of Formula (IA) is AMG1063. In some embodiments, the compound of Formula (IA) is AMG1064. In some embodiments, the compound of Formula (IA) is AMG1065. In some embodiments, the compound of Formula (IA) is AMG1066. In some embodiments, the compound of Formula (IA) is AMG1067. In some embodiments, the compound of Formula (IA) is AMG1068.

### Pharmaceutical Compositions and Administration

The present disclosure provides compositions (e.g., pharmaceutical compositions) comprising a compound provided herein (e.g., a compound of Formula (I)), or a pharmaceutically acceptable salt thereof, and an excipient (e.g., a pharmaceutically acceptable excipient). In one aspect, the composition is a pharmaceutical composition, and the excipient is a pharmaceutically acceptable excipient.

In some embodiments, the composition further comprises an agent.

In certain embodiments, the compound described herein is provided in an effective amount in the pharmaceutical composition. In certain embodiments, the effective amount is an amount effective for delivering an agent to a subject or cell. In certain embodiments, the effective amount is an amount effective for delivering a polynucleotide to a subject or cell. In certain embodiments, the effective amount is an amount effective for delivering mRNA to a subject or cell. In certain embodiments, the effective amount is an amount effective for chimeric antigen receptor T cell therapy.

In certain embodiments, the composition further comprises one or more of a PEG-lipid, sterol, or a phospholipid. In certain embodiments, the composition comprises a PEG-lipid. In some embodiments, the composition comprises a sterol. In certain embodiments, the composition comprises a phospholipid. In certain embodiments, the composition further comprises a PEG-lipid and a sterol. In certain embodiments, the composition further comprises a PEG-lipid and a phospholipid. In certain embodiments, the composition further comprises a sterol and a phospholipid. In certain embodiments, the composition comprises one or more of a PEG-lipid, sterol, or a phospholipid and is formulated as a particle. In some embodiments, the composition comprises one or more of a PEG-lipid, sterol, or a phospholipid and is formulated as a nanoparticle or microparticle. In certain embodiments, the composition comprises one or more of a PEG-lipid, sterol, or a phospholipid and is formulated as a lipid nanoparticle. In certain embodiments, the composition comprises one or more of a PEG-lipid, sterol, or a phospholipid and is formulated as a micelle, liposome, or lipoplex.

In some embodiments, the composition comprises approximately 20-70 molar % of a compound of Formula (I), or a pharmaceutically acceptable salt thereof. In some embodiments, the composition comprises approximately 25-65 molar % of a compound of Formula (I), or a pharmaceutically acceptable salt thereof. In some embodiments, the composition comprises approximately 20-60 molar % of a compound of Formula (I), or a pharmaceutically acceptable salt thereof. In some embodiments, the composition comprises approximately 30-60 molar % of a compound of Formula (I), or a pharmaceutically acceptable salt thereof. In some embodiments, the composition comprises approximately 35-60 molar % of a compound of Formula (I), or a pharmaceutically acceptable salt thereof. In some embodiments, the composition comprises approximately 40-60 molar % of a compound of Formula (I), or a pharmaceutically acceptable salt thereof. In some embodiments, the composition comprises approximately 45-60 molar % of a compound of Formula (I), or a pharmaceutically acceptable salt thereof. In some embodiments, the composition comprises approximately 50-60 molar % of a compound of Formula (I), or a pharmaceutically acceptable salt thereof. In some embodiments, the composition comprises approximately 55-60 molar % of a compound of Formula (I), or a pharmaceutically acceptable salt thereof. In some embodiments, the composition comprises approximately 20-30 molar % of a compound of Formula (I), or a pharmaceutically acceptable salt thereof. In some embodiments, the composition comprises approximately 20-35 molar % of a compound of Formula (I), or a pharmaceutically acceptable salt thereof. In some embodiments, the composition comprises approximately 20-40 molar % of a compound of Formula (I), or a pharmaceutically acceptable salt thereof. In some embodiments, the composition comprises approximately 20-45 molar % of a compound of Formula (I), or a pharmaceutically acceptable salt thereof. In some embodiments, the composition comprises approximately 20-50 molar % of a compound of Formula (I), or a pharmaceutically acceptable salt thereof. In some embodiments, the composition comprises approximately 20-55 molar % of a compound of Formula (I), or a pharmaceutically acceptable salt thereof. In some embodiments, the composition comprises approximately 30, approximately 35, approximately 40, approximately 45, approximately 50, approximately 55, or approximately 60 molar % of a compound of Formula (I), or a pharmaceutically acceptable salt thereof.

In some embodiments, the composition comprises approximately 0-75 molar % of a sterol. In some embodiments, the composition comprises approximately 5-60 molar % of a sterol. In some embodiments, the composition comprises approximately 5-50 molar % of a sterol. In some embodiments, the composition comprises approximately 5-40 molar % of a sterol. In some embodiments, the composition comprises approximately 5-30 molar % of a sterol. In some embodiments, the composition comprises approximately 5-20 molar % of a sterol. In some embodiments, the composition comprises approximately 10-60 molar % of a sterol. In some embodiments, the composition comprises approximately 20-60 molar % of a sterol. In some embodiments, the composition comprises approximately 30-60 molar % of a sterol. In some embodiments, the composition comprises approximately 40-60 molar % of a sterol. In some embodiments, the composition comprises approximately 50-60 molar % of a sterol. In some embodiments, the composition comprises approximately 20-55 molar % of a sterol. In some embodiments, the composition comprises approximately 25-50 molar % of a sterol. In some embodiments, the composition comprises approximately 5, approximately 7, approximately 8.5, approximately 10, approximately 12.5, approximately 15, approximately 17, approximately 20, approximately 23.5, approximately 25, approximately 27.75, approximately 28.5, approximately 30, approximately 32.75, approximately 35, approximately 37, approximately 37.75, approximately 38.5, approximately 40, approximately 42, approximately 45, approximately 48.5, approximately 50, approximately 55, approximately 57, approximately 58.5, or approximately 60 molar % of a sterol. In some embodiments, the composition comprises approximately 7, approximately 8.5, approximately 17, approximately 23.5, approximately 27.75, approximately 28.5, approximately 32.75, approximately 37, approximately 37.75, approximately 38.5, approximately 42, approximately 48.5, approximately 57, or approximately 58.5 molar % of a sterol.

In some embodiments, the composition comprises approximately 0-75 molar % of cholesterol. In some embodiments, the composition comprises approximately 5-60 molar % of cholesterol. In some embodiments, the composition comprises approximately 5-50 molar % of cholesterol. In some embodiments, the composition comprises approximately 5-40 molar % of cholesterol. In some embodiments, the composition comprises approximately 5-30 molar % of cholesterol. In some embodiments, the composition comprises approximately 5-20 molar % of cholesterol. In some embodiments, the composition comprises approximately 10-60 molar % of cholesterol. In some embodiments, the composition comprises approximately 20-60 molar % of cholesterol. In some embodiments, the composition comprises approximately 30-60 molar % of cholesterol. In some embodiments, the composition comprises approximately 40-60 molar % of cholesterol. In some embodiments, the composition comprises approximately 50-60 molar % of cholesterol. In some embodiments, the composition comprises approximately 20-55 molar % of cholesterol. In some embodiments, the composition comprises approximately 25-50 molar % of cholesterol. In some embodiments, the composition comprises approximately 5, approximately 7, approximately 8.5, approximately 10, approximately 12.5, approximately 15, approximately 17, approximately 20, approximately 23.5, approximately 25, approximately 27.75, approximately 28.5, approximately 30, approximately 32.75, approximately 35, approximately 37, approximately 37.75, approximately 38.5, approximately 40, approximately 42, approximately 45, approximately 48.5, approximately 50, approximately 55, approximately 57, approximately 58.5, or approximately 60 molar % of cholesterol. In some embodiments, the composition comprises approximately 7, approximately 8.5, approximately 17, approximately 23.5, approximately 27.75, approximately 28.5, approximately 32.75, approximately 37, approximately 37.75, approximately 38.5, approximately 42, approximately 48.5, approximately 57, or approximately 58.5 molar % of cholesterol.

In some embodiments, the composition comprises approximately 0-20 molar % of a PEG-lipid. In some embodiments, the composition comprises approximately 0-15 molar % of a PEG-lipid. In some embodiments, the composition comprises approximately 0-10 molar % of a PEG-lipid. In some embodiments, the composition comprises approximately 0-5 molar % of a PEG-lipid. In some embodiments, the composition comprises approximately 1-20 molar % of a PEG-lipid. In some embodiments, the composition comprises approximately 1-15 molar % of a PEG-lipid. In some embodiments, the composition comprises approximately 1-10 molar % of a PEG-lipid. In some embodiments, the composition comprises approximately 1-5 molar % of a PEG-lipid. In some embodiments, the composition comprises approximately 1-3 molar % of a PEG-lipid. In some embodiments, the composition comprises approximately 0.5, approximately 0.75, approximately 1.0, approximately 1.25, approximately 1.5, approximately 1.75, approximately 2.0, approximately 2.25, approximately 2.5, approximately 2.75, approximately 3.0, approximately 3.25, or approximately 3.5 molar % of a PEG-lipid.

In some embodiments, the composition comprises approximately 0-20 molar % of DMPE-PEG2k. In some embodiments, the composition comprises approximately 0-15 molar % of DMPE-PEG2k. In some embodiments, the composition comprises approximately 0-10 molar % of DMPE-PEG2k. In some embodiments, the composition comprises approximately 0-5 molar % of DMPE-PEG2k. In some embodiments, the composition comprises approximately 1-20 molar % of DMPE-PEG2k. In some embodiments, the composition comprises approximately 1-15 molar % of DMPE-PEG2k. In some embodiments, the composition comprises approximately 1-10 molar % of DMPE-PEG2k. In some embodiments, the composition comprises approximately 1-5 molar % of DMPE-PEG2k. In some embodiments, the composition comprises approximately 1-3 molar % of DMPE-PEG2k. In some embodiments, the composition comprises approximately 0.5, approximately 0.75, approximately 1.0, approximately 1.25, approximately 1.5, approximately 1.75, approximately 2.0, approximately 2.25, approximately 2.5, approximately 2.75, approximately 3.0, approximately 3.25, or approximately 3.5 molar % of DMPE-PEG2k.

In some embodiments, the composition comprises approximately 0-50 molar % of a phospholipid. In some embodiments, the composition comprises approximately 0-40 molar % of a phospholipid. In some embodiments, the composition comprises approximately 10-15 molar % of a phospholipid. In some embodiments, the composition comprises approximately 10-20 molar % of a phospholipid. In some embodiments, the composition comprises approximately 10-25 molar % of a phospholipid. In some embodiments, the composition comprises approximately 10-30 molar % of a phospholipid. In some embodiments, the composition comprises approximately 10-35 molar % of a phospholipid. In some embodiments, the composition comprises approximately 10-40 molar % of a phospholipid. In some embodiments, the composition comprises approximately 10-45 molar % of a phospholipid. In some embodiments, the composition comprises approximately 10-50 molar % of a phospholipid. In some embodiments, the composition comprises approximately 5-45 molar % of a phospholipid. In some embodiments, the composition comprises approximately 15-45 molar % of a phospholipid. In some embodiments, the composition comprises approximately 20-45 molar % of a phospholipid. In some embodiments, the composition comprises approximately 25-45 molar % of a phospholipid. In some embodiments, the composition comprises approximately 5, approximately 10, approximately 15, approximately 20, approximately 30, approximately 35, approximately 40, approximately 45, or approximately 50 molar % of a phospholipid.

In some embodiments, the composition comprises approximately 0-50 molar % of DOPE. In some embodiments, the composition comprises approximately 0-40 molar % of DOPE. In some embodiments, the composition comprises approximately 10-15 molar % of DOPE. In some embodiments, the composition comprises approximately 10-20 molar % of DOPE. In some embodiments, the composition comprises approximately 10-25 molar % of DOPE. In some embodiments, the composition comprises approximately 10-30 molar % of DOPE. In some embodiments, the composition comprises approximately 10-35 molar % of DOPE. In some embodiments, the composition comprises approximately 10-40 molar % of DOPE. In some embodiments, the composition comprises approximately 10-45 molar % of DOPE. In some embodiments, the composition comprises approximately 10-50 molar % of DOPE. In some embodiments, the composition comprises approximately 5-45 molar % of DOPE. In some embodiments, the composition comprises approximately 15-45 molar % of DOPE. In some embodiments, the composition comprises approximately 20-45 molar % of DOPE. In some embodiments, the composition comprises approximately 25-45 molar % of DOPE. In some embodiments, the composition comprises approximately 5, approximately 10, approximately 15, approximately 20, approximately 30, approximately 35, approximately 40, approximately 45, or approximately 50 molar % of DOPE.

In some embodiments, the composition comprises approximately 0-50 molar % of DSPC. In some embodiments, the composition comprises approximately 0-40 molar % of DSPC. In some embodiments, the composition comprises approximately 10-15 molar % of DSPC. In some embodiments, the composition comprises approximately 10-20 molar % of DSPC. In some embodiments, the composition comprises approximately 10-25 molar % of DSPC. In some embodiments, the composition comprises approximately 10-30 molar % of DSPC. In some embodiments, the composition comprises approximately 10-35 molar % of DSPC. In some embodiments, the composition comprises approximately 10-40 molar % of DSPC. In some embodiments, the composition comprises approximately 10-45 molar % of DSPC. In some embodiments, the composition comprises approximately 10-50 molar % of DSPC. In some embodiments, the composition comprises approximately 5-45 molar % of DSPC. In some embodiments, the composition comprises approximately 15-45 molar % of DSPC. In some embodiments, the composition comprises approximately 20-45 molar % of DSPC. In some embodiments, the composition comprises approximately 25-45 molar % of DSPC. In some embodiments, the composition comprises approximately 5, approximately 10, approximately 15, approximately 20, approximately 30, approximately 35, approximately 40, approximately 45, or approximately 50 molar % of DSPC.

In some embodiments, the composition comprises about a 20:1 ratio of a compound of Formula (I), or a pharmaceutically acceptable salt thereof: agent by mass. In some embodiments, the composition comprises about a 17.5:1 ratio of a compound of Formula (I), or a pharmaceutically acceptable salt thereof: agent by mass. In some embodiments, the composition comprises about a 15:1 ratio of a compound of Formula (I), or a pharmaceutically acceptable salt thereof: agent by mass. In some embodiments, the composition comprises about a 12.5:1 ratio of a compound of Formula (I), or a pharmaceutically acceptable salt thereof: agent by mass. In some embodiments, the composition comprises about a 10:1 ratio of a compound of Formula (I), or a pharmaceutically acceptable salt thereof: agent by mass. In some embodiments, the composition comprises about a 7.5:1 ratio of a compound of Formula (I), or a pharmaceutically acceptable salt thereof: agent by mass. In some embodiments, the composition comprises about a 5:1 ratio of a compound of Formula (I), or a pharmaceutically acceptable salt thereof: agent by mass. In some embodiments, the composition comprises about a 2.5:1 ratio of a compound of Formula (I), or a pharmaceutically acceptable salt thereof: agent by mass.

In some embodiments, the composition comprises about a 20:1 ratio of a compound of Formula (I), or a pharmaceutically acceptable salt thereof: polynucleotide by mass. In some embodiments, the composition comprises about a 17.5:1 ratio of a compound of Formula (I), or a pharmaceutically acceptable salt thereof: polynucleotide by mass. In some embodiments, the composition comprises about a 15:1 ratio of a compound of Formula (I), or a pharmaceutically acceptable salt thereof: polynucleotide by mass. In some embodiments, the composition comprises about a 12.5:1 ratio of a compound of Formula (I), or a pharmaceutically acceptable salt thereof: polynucleotide by mass. In some embodiments, the composition comprises about a 10:1 ratio of a compound of Formula (I), or a pharmaceutically acceptable salt thereof: polynucleotide by mass. In some embodiments, the composition comprises about a 7.5:1 ratio of a compound of Formula (I), or a pharmaceutically acceptable salt thereof: polynucleotide by mass. In some embodiments, the composition comprises about a 5:1 ratio of a compound of Formula (I), or a pharmaceutically acceptable salt thereof: polynucleotide by mass. In some embodiments, the composition comprises about a 2.5:1 ratio of a compound of Formula (I), or a pharmaceutically acceptable salt thereof: polynucleotide by mass.

In some embodiments, the composition comprises about a 20:1 ratio of a compound of Formula (I), or a pharmaceutically acceptable salt thereof: mRNA by mass. In some embodiments, the composition comprises about a 17.5:1 ratio of a compound of Formula (I), or a pharmaceutically acceptable salt thereof: mRNA by mass. In some embodiments, the composition comprises about a 15:1 ratio of a compound of Formula (I), or a pharmaceutically acceptable salt thereof: mRNA by mass. In some embodiments, the composition comprises about a 12.5:1 ratio of a compound of Formula (I), or a pharmaceutically acceptable salt thereof: mRNA by mass. In some embodiments, the composition comprises about a 10:1 ratio of a compound of Formula (I), or a pharmaceutically acceptable salt thereof: mRNA by mass. In some embodiments, the composition comprises about a 7.5:1 ratio of a compound of Formula (I), or a pharmaceutically acceptable salt thereof: mRNA by mass. In some embodiments, the composition comprises about a 5:1 ratio of a compound of Formula (I), or a pharmaceutically acceptable salt thereof: mRNA by mass. In some embodiments, the composition comprises about a 2.5:1 ratio of a compound of Formula (I), or a pharmaceutically acceptable salt thereof: mRNA by mass.

In some embodiments, the composition comprises a compound of Formula (I), or a pharmaceutically acceptable salt thereof, a phospholipid, a PEG lipid, and a sterol. In some embodiments, the composition comprises a compound of Formula (I), or a pharmaceutically acceptable salt thereof, DOPE, DMPE-PEG2k, and cholesterol. In some embodiments, the composition comprises a compound of Formula (I), or a pharmaceutically acceptable salt thereof, DSPC, DMPE-PEG2k, and cholesterol.

In some embodiments, the composition comprises approximately 30-60% a compound of Formula (I), or a pharmaceutically acceptable salt thereof, approximately 10-30% of a phospholipid, approximately 1.5-3% of a PEG lipid, and approximately 7-58.5% a sterol. In some embodiments, the composition comprises approximately 30-60% a compound of Formula (I), or a pharmaceutically acceptable salt thereof, approximately 10-30% of DOPE, approximately 1.5-3% of DMPE-PEG2k, and approximately 7-58.5% cholesterol. In some embodiments, the composition comprises approximately 30-60% a compound of Formula (I), or a pharmaceutically acceptable salt thereof, approximately 10-30% of DSPC, approximately 1.5-3% of DMPE-PEG2k, and approximately 7-58.5% cholesterol.

In some embodiments, the composition comprises approximately 20-30% of a phospholipid, approximately 1.5-3% of a PEG-lipid, approximately 30% of a compound of Formula (I), or a pharmaceutically acceptable salt thereof, and approximately 37-48.5% of a sterol. In some embodiments, the composition comprises approximately 30% of a phospholipid, approximately 3% of a PEG-lipid, approximately 30% of a compound of Formula (I), or a pharmaceutically acceptable salt thereof, and approximately 37% of a sterol. In some embodiments, the composition comprises approximately 20% of a phospholipid, approximately 1.5% of a PEG-lipid, approximately 30% of a compound of Formula (I), or a pharmaceutically acceptable salt thereof, and approximately 48.5% of a sterol. In some embodiments, the composition comprises approximately 30% of a phospholipid, approximately 2.25% of a PEG-lipid, approximately 30% of a compound of Formula (I), or a pharmaceutically acceptable salt thereof, and approximately 37.75% of a sterol.

In some embodiments, the composition comprises approximately 20-30% of DOPE, approximately 1.5-3% of DMPE-PEG-2k, approximately 30% of a compound of Formula (I), or a pharmaceutically acceptable salt thereof, and approximately 37-48.5% of cholesterol. In some embodiments, the composition comprises approximately 30% of DOPE, approximately 3% of DMPE-PEG-2k, approximately 30% of a compound of Formula (I), or a pharmaceutically acceptable salt thereof, and approximately 37% of cholesterol. In some embodiments, the composition comprises approximately 20% of DOPE, approximately 1.5% of DMPE-PEG-2k, approximately 30% of a compound of Formula (I), or a pharmaceutically acceptable salt thereof, and approximately 48.5% of cholesterol. In some embodiments, the composition comprises approximately 30% of DOPE, approximately 2.25% of DMPE-PEG-2k, approximately 30% of a compound of Formula (I), or a pharmaceutically acceptable salt thereof, and approximately 37.75% of cholesterol.

In some embodiments, the composition comprises approximately 10-30% of a phospholipid, approximately 2.25-3% of a PEG-lipid, approximately 30-45% of a compound of Formula (I), or a pharmaceutically acceptable salt thereof, and approximately 32.75-57% of a sterol. In some embodiments, the composition comprises approximately 20% of a phospholipid, approximately 2.25% of a PEG-lipid, approximately 45% of a compound of Formula (I), or a pharmaceutically acceptable salt thereof, and approximately 32.75% of a sterol. In some embodiments, the composition comprises approximately 30% of a phospholipid, approximately 2.25% of a PEG-lipid, approximately 30% of a compound of Formula (I), or a pharmaceutically acceptable salt thereof, and approximately 37.75% of a sterol. In some embodiments, the composition comprises approximately 10% of a phospholipid, approximately 3% of a PEG-lipid, approximately 30% of a compound of Formula (I), or a pharmaceutically acceptable salt thereof, and approximately 57% of a sterol.

In some embodiments, the composition comprises approximately 10-30% of DSPC, approximately 2.25-3% of DMPE-PEG-2k, approximately 30-45% of a compound of Formula (I), or a pharmaceutically acceptable salt thereof, and approximately 32.75-57% of cholesterol. In some embodiments, the composition comprises approximately 20% of DSPC, approximately 2.25% of DMPE-PEG-2k, approximately 45% of a compound of Formula (I), or a pharmaceutically acceptable salt thereof, and approximately 32.75% of cholesterol. In some embodiments, the composition comprises approximately 30% of DSPC, approximately 2.25% of DMPE-PEG-2k, approximately 30% of a compound of Formula (I), or a pharmaceutically acceptable salt thereof, and approximately 37.75% of cholesterol. In some embodiments, the composition comprises approximately 10% of DSPC, approximately 3% of DMPE-PEG-2k, approximately 30% of a compound of Formula (I), or a pharmaceutically acceptable salt thereof, and approximately 57% of cholesterol.

In some embodiments, the composition comprises approximately 20-30% a compound of Formula (I), or a pharmaceutically acceptable salt thereof, approximately 30-40% of a phospholipid, approximately 2.5% of a PEG lipid, and approximately 27.5-47.5% a sterol.

In some embodiments, the composition comprises approximately 30-40% of a phospholipid, approximately 25-30% of a compound of Formula (I), or a pharmaceutically acceptable salt thereof, approximately 2.5% of a PEG lipid, and approximately 27.5-37.5% of a sterol. In some embodiments, the composition comprises approximately 35% of a phospholipid, approximately 30% of a compound of Formula (I), or a pharmaceutically acceptable salt thereof, approximately 2.5% of a PEG lipid, and approximately 32.5% of a sterol. In some embodiments, the composition comprises approximately 30% of a phospholipid, approximately 30% of a compound of Formula (I), or a pharmaceutically acceptable salt thereof, approximately 2.5% of a PEG lipid, and approximately 37.5% of a sterol. In some embodiments, the composition comprises approximately 40% of a phospholipid, approximately 30% of a compound of Formula (I), or a pharmaceutically acceptable salt thereof, approximately 2.5% of a PEG lipid, and approximately 27.5% of a sterol. In some embodiments, the composition comprises approximately 40% of a phospholipid, approximately 25% of a compound of Formula (I), or a pharmaceutically acceptable salt thereof, approximately 2.5% of a PEG lipid, and approximately 32.5% of a sterol.

In some embodiments, the composition comprises approximately 30-40% of a phospholipid, approximately 25-30% of a compound of Formula (I), or a pharmaceutically acceptable salt thereof, approximately 2.5% of a DMPE-PEG2k, and approximately 27.5-37.5% of cholesterol. In some embodiments, the composition comprises approximately 35% of a phospholipid, approximately 30% of a compound of Formula (I), or a pharmaceutically acceptable salt thereof, approximately 2.5% of a DMPE-PEG2k, and approximately 32.5% of cholesterol. In some embodiments, the composition comprises approximately 30% of a phospholipid, approximately 30% of a compound of Formula (I), or a pharmaceutically acceptable salt thereof, approximately 2.5% of a DMPE-PEG2k, and approximately 37.5% of cholesterol. In some embodiments, the composition comprises approximately 40% of a phospholipid, approximately 30% of a compound of Formula (I), or a pharmaceutically acceptable salt thereof, approximately 2.5% of a DMPE-PEG2k, and approximately 27.5% of cholesterol. In some embodiments, the composition comprises approximately 40% of a phospholipid, approximately 25% of a compound of Formula (I), or a pharmaceutically acceptable salt thereof, approximately 2.5% of a DMPE-PEG2k, and approximately 32.5% of cholesterol.

In some embodiments, the composition comprises approximately 30-40% of DSPC, approximately 25-30% of a compound of Formula (I), or a pharmaceutically acceptable salt thereof, approximately 2.5% of a DMPE-PEG2k, and approximately 27.5-37.5% of cholesterol. In some embodiments, the composition comprises approximately 35% of DSPC, approximately 30% of a compound of Formula (I), or a pharmaceutically acceptable salt thereof, approximately 2.5% of a DMPE-PEG2k, and approximately 32.5% of cholesterol. In some embodiments, the composition comprises approximately 30% of DSPC, approximately 30% of a compound of Formula (I), or a pharmaceutically acceptable salt thereof, approximately 2.5% of a DMPE-PEG2k, and approximately 37.5% of cholesterol. In some embodiments, the composition comprises approximately 40% of DSPC, approximately 30% of a compound of Formula (I), or a pharmaceutically acceptable salt thereof, approximately 2.5% of a DMPE-PEG2k, and approximately 27.5% of cholesterol. In some embodiments, the composition comprises approximately 40% of DSPC, approximately 25% of a compound of Formula (I), or a pharmaceutically acceptable salt thereof, approximately 2.5% of a DMPE-PEG2k, and approximately 32.5% of cholesterol.

In some embodiments, the composition comprises approximately 30-40% of DOPE, approximately 25-30% of a compound of Formula (I), or a pharmaceutically acceptable salt thereof, approximately 2.5% of a DMPE-PEG2k, and approximately 27.5-37.5% of cholesterol. In some embodiments, the composition comprises approximately 35% of DOPE, approximately 30% of a compound of Formula (I), or a pharmaceutically acceptable salt thereof, approximately 2.5% of a DMPE-PEG2k, and approximately 32.5% of cholesterol. In some embodiments, the composition comprises approximately 30% of DOPE, approximately 30% of a compound of Formula (I), or a pharmaceutically acceptable salt thereof, approximately 2.5% of a DMPE-PEG2k, and approximately 37.5% of cholesterol. In some embodiments, the composition comprises approximately 40% of DOPE, approximately 30% of a compound of Formula (I), or a pharmaceutically acceptable salt thereof, approximately 2.5% of a DMPE-PEG2k, and approximately 27.5% of cholesterol. In some embodiments, the composition comprises approximately 40% of DOPE, approximately 25% of a compound of Formula (I), or a pharmaceutically acceptable salt thereof, approximately 2.5% of a DMPE-PEG2k, and approximately 32.5% of cholesterol.

In some embodiments, the composition comprises approximately 35% of a compound of Formula (I), or a pharmaceutically acceptable salt thereof, approximately 16% of a phospholipid, approximately 46.5% of a sterol, and approximately 2.5% of a PEG-lipid. In some embodiments, the composition comprises approximately 35% of a compound of Formula (I), or a pharmaceutically acceptable salt thereof, approximately 16% of DOPE, approximately 46.5% of cholesterol, and approximately 2.5% of DMPE-PEG.

Pharmaceutical compositions described herein can be prepared by any method known in the art of pharmaceutics. In general, such preparatory methods include bringing a compound, agent, or particle described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents such as calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor*, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (II) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound, agent, or particle described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound, agent, or particle in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds, agents, or particles provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound, agent, or particle required to achieve an effective amount will vary from subject to subject, depending, for example, on species, age, and general condition of a subject, severity of the side effects or disorder, identity of the particular compound, agent or particle, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound, agent, or particle described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound, agent, or particle described herein. In certain embodiments, a dose described herein includes independently between 1 mg and 3 mg, inclusive, of a compound, agent, or particle described herein. In certain embodiments, a dose described herein includes independently between 3 mg and 10 mg, inclusive, of a compound, agent, or particle described herein. In certain embodiments, a dose described herein includes independently between 10 mg and 30 mg, inclusive, of a compound, agent, or particle described herein. In certain embodiments, a dose described herein includes independently between 30 mg and 100 mg, inclusive, of a compound, agent, or particle described herein.

In certain embodiments, a dose (e.g., a single dose, or any dose of multiple doses) described herein includes independently between 0.001 mg/kg and 25 mg/kg, between 0.01 mg/kg and 10 mg/kg, between 0.05 mg/kg and 5 mg/kg, between 0.1 mg/kg and 2.5 mg/kg, between 0.1 mg/kg and 1 mg/kg, between 0.2 mg/kg and 0.8 mg/kg, or between 0.25 mg/kg and 0.75 mg/kg, inclusive, of a compound, agent, or particle described herein. In certain embodiments, a dose described herein includes independently between 0.1 mg/kg and 0.1 mg/kg, inclusive, of a compound, agent, or particle described herein. In certain embodiments, a dose described herein includes independently between 0.1 mg/kg and 1 mg/kg, inclusive, of a compound, agent, or particle described herein. In certain embodiments, a dose described herein includes independently between 0.1 mg/kg and 2.5 mg/kg, inclusive, of a compound, agent, or particle described herein. In certain embodiments, a dose described herein includes independently between 2.5 mg/kg and 5 mg/kg, inclusive, of a compound, agent, or particle described herein. In certain embodiments, a dose described herein includes independently between 5 mg/kg and 25 mg/kg, inclusive, of a compound, agent, or particle described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that treat a disease in a subject in need thereof, prevent a disease in a subject in need thereof, or reduce the risk to develop a disease in a subject in need thereof, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, polynucleotides, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., lung disease or liver disease). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

### PEG-Lipid

In some embodiments, the PEG-lipid is a PEG-phospholipid or PEG-glyceride lipid.

In certain embodiments, the PEG-lipid is a PEG-phospholipid. In certain embodiments, the PEG-phospholipid is a PEG-phosphoethanolamine. In some embodiments, the PEG-phospholipid is a PEG-phosphatidylcholine.

In certain embodiments, the PEG component has a molecular weight of about 350, about 550, about 750, about 1000, about 2000, about 3000, or about 5000. In some embodiments, the PEG component has a molecular weight of about 750, about 1000, about 2000, about 3000, or about 5000. In certain embodiments, the PEG component has a molecular weight of 500-1000, 1000-2000, 2000-3000, or 3000-4000. In certain embodiments, the PEG component has a molecular weight of about 1000, about 2000, or about 3000. In some embodiments, the PEG component has a molecular weight of about 2000.

In certain embodiments, the PEG-lipid is stearoyl-substituted (Cis). In some embodiments, the PEG-phospholipid is palmitoyl-substituted (C₁₆). In certain embodiments, the PEG-phospholipid is myristoyl-substituted (C₁₄).

In certain embodiments, the PEG-lipid is selected from the group consisting of 1,2-distearoyl-sn-glycero-3-phosphoethanolamine-N-[methoxy(polyethylene glycol)-5000](C₁₈PEG5000), 1,2-dipalmitoyl-sn-glycero-3-phosphoethanolamine-N-[methoxy(polyethylene glycol)-5000] (C₁₆PEG5000), 1,2-dimyristoyl-sn-glycero-3-phosphoethanolamine-N-[methoxy(polyethylene glycol)-5000] (C₁₄PEG5000), 1,2-distearoyl-sn-glycero-3-phosphoethanolamine-N-[methoxy(polyethylene glycol)-3000](C₁₈PEG3000), 1,2-dipalmitoyl-sn-glycero-3-phosphoethanolamine-N-[methoxy(polyethylene glycol)-3000] (C₁₆PEG3000), 1,2-dimyristoyl-sn-glycero-3-phosphoethanolamine-N-[methoxy(polyethylene glycol)-3000] (C₁₄PEG3000), 1,2-distearoyl-sn-glycero-3-phosphoethanolamine-N-[methoxy(polyethylene glycol)-2000](C₁₈PEG2000), 1,2-dipalmitoyl-sn-glycero-3-phosphoethanolamine-N-[methoxy(polyethylene glycol)-2000] (C₁₆PEG2000), 1,2-dimyristoyl-sn-glycero-3-phosphoethanolamine-N-[methoxy(polyethylene glycol)-2000] (C₁₄PEG2000), 1,2-distearoyl-sn-glycero-3-phosphoethanolamine-N-[methoxy(polyethylene glycol)-1000](C₁₈PEG1000), 1,2-dipalmitoyl-sn-glycero-3-phosphoethanolamine-N-[methoxy(polyethylene glycol)-1000] (C₁₆PEG1000), 1,2-dimyristoyl-sn-glycero-3-phosphoethanolamine-N-[methoxy(polyethylene glycol)-1000] (C₁₄PEG1000), 1,2-distearoyl-sn-glycero-3-phosphoethanolamine-N-[methoxy(polyethylene glycol)-750](C₁₈PEG750), 1,2-dipalmitoyl-sn-glycero-3-phosphoethanolamine-N-[methoxy(polyethylene glycol)-750] (C₁₆PEG750), and 1,2-dimyristoyl-sn-glycero-3-phosphoethanolamine-N-[methoxy(polyethylene glycol)-750] (C₁₄PEG750). In some embodiments, the PEG-lipid is selected from the group consisting of 1,2-dimyristoyl-sn-glycero-3-phosphoethanolamine-polyethylene glycol (DMPE-PEG), 1,2-distearoyl-sn-glycero-3-phosphoethanolamine-N-[methoxy(polyethylene glycol)-2000] (C₁₈PEG2000), 1,2-dipalmitoyl-sn-glycero-3-phosphoethanolamine-N-[methoxy(polyethylene glycol)-2000] (C₁₆PEG2000), and 1,2-dimyristoyl-sn-glycero-3-phosphoethanolamine-N-[methoxy(polyethylene glycol)-2000](C₁₄PEG2000). In certain embodiments, the PEG-lipid is selected from the group consisting of 1,2-dimyristoyl-sn-glycero-3-phosphoethanolamine-polyethylene glycol (DMPE-PEG), 1,2-dimyristoyl-sn-glycero-3-phosphoethanolamine-N-[methoxy(polyethylene glycol)-5000](C₁₄PEG5000), 1,2-dimyristoyl-sn-glycero-3-phosphoethanolamine-N-[methoxy(polyethylene glycol)-3000] (C₁₄PEG3000), 1,2-dimyristoyl-sn-glycero-3-phosphoethanolamine-N-[methoxy(polyethylene glycol)-2000] (C₁₄PEG2000), 1,2-dimyristoyl-sn-glycero-3-phosphoethanolamine-N-[methoxy(polyethylene glycol)-1000](C₁₄PEG1000), and 1,2-dimyristoyl-sn-glycero-3-phosphoethanolamine-N-[methoxy(polyethylene glycol)-750] (C₁₄PEG750). In certain embodiments, the PEG-phospholipid is 1,2-dimyristoyl-sn-glycero-3-phosphoethanolamine-N-[methoxy(polyethylene glycol)-2000] (C₁₄PEG2000). In some embodiments, the PEG-lipid is 1,2-dimyristoyl-sn-glycero-3-phosphoethanolamine-polyethylene glycol (DMPE-PEG).

In some embodiments, the PEG-lipid is a PEG-glyceride lipid. In certain embodiments, the PEG-lipid is 1,2-distearoyl-rac-glycero-3-methoxypolyethylene glycol-2000 (DSG-PEG2000) or 1,2-dimyristoyl-rac-glycero-3-methoxypolyethylene glycol-2000 (DMG-PEG2000). In some embodiments, the PEG-lipid is 1,2-distearoyl-rac-glycero-3-methoxypolyethylene glycol-2000 (DSG-PEG2000). In certain embodiments, the PEG-lipid is 1,2-dimyristoyl-rac-glycero-3-methoxypolyethylene glycol-2000 (DMG-PEG2000).

### Sterol

In certain embodiments, the sterol is cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, or a derivative thereof. In some embodiments, the sterol is cholesterol, or a derivative thereof. In certain embodiments, the sterol is cholesterol.

### Phospholipid

In certain embodiments, the phospholipid is a phosphoethanolamine or phosphatidylcholine. In some embodiments, the phospholipid is 1,2-distearoyl-sn-glycero-3-phosphorylethanolamine (DSPE), 1,2-distearoyl-sn-glycero-3-phosphocholine (DSPC), or 1,2-dioleoyl-sn-glycero-3-phosphoethanolamine (DOPE). In some embodiments, the phospholipid is a phosphoethanolamine. In certain embodiments, the phospholipid is 1,2-distearoyl-sn-glycero-3-phosphorylethanolamine (DSPE) or 1,2-dioleoyl-sn-glycero-3-phosphoethanolamine (DOPE). In certain embodiments, the phospholipid is 1,2-dioleoyl-sn-glycero-3-phosphoethanolamine (DOPE) or distearoylphosphatidylcholine (DSPC). In certain embodiments, the phospholipid is 1,2-distearoyl-sn-glycero-3-phosphorylethanolamine (DSPE). In certain embodiments, the phospholipid is 1,2-dioleoyl-sn-glycero-3-phosphoethanolamine (DOPE). In some embodiments, the phospholipid is a phosphatidylcholine. In some embodiments, the phospholipid is 1,2-distearoyl-sn-glycero-3-phosphocholine (DSPC).

### Agents

In certain embodiments, the composition further comprises an agent. In some embodiments, the agent is an organic molecule, inorganic molecule, polynucleotide, protein, peptide, targeting agent, an isotopically labeled chemical compound, vaccine, an immunological agent, or an agent useful in bioprocessing.

In some embodiments, the agent and the compound, or the pharmaceutically acceptable salt thereof, are not covalently attached.

Agents that are delivered by the systems (e.g., pharmaceutical compositions) described herein may be (e.g., therapeutic or prophylactic), diagnostic, cosmetic, or nutraceutical agents. Any chemical compound to be administered to a subject may be delivered using the complexes, picoparticles, nanoparticles (e.g., lipid nanoparticles), microparticles, micelles, or liposomes, described herein. In some embodiments, the agent is an organic molecule, inorganic molecule, protein, peptide, polynucleotide, targeting agent, an isotopically labeled chemical compound, vaccine, an immunological agent, or an agent useful in bioprocessing (e.g., intracellular manufacturing of proteins, such as a cell's bioprocessing of a commercially useful chemical or fuel). For example, intracellular delivery of an agent may be useful in bioprocessing by maintaining the cell's health and/or growth, e.g., in the manufacturing of proteins. Any chemical compound to be administered to a subject or contacted with a cell may be delivered to the subject or cell using the compositions.

Exemplary agents that may be included in a composition described herein include, but are not limited to, small molecules, organometallic compounds, polynucleotides, proteins, peptides, carbohydrates, monosaccharides, oligosaccharides, polysaccharides, nucleoproteins, mucoproteins, lipoproteins, small molecules linked to proteins, glycoproteins, steroids, nucleotides, oligonucleotides, polynucleotides, nucleosides, antisense oligonucleotides, lipids, hormones, vitamins, cells, metals, targeting agents, isotopically labeled chemical compounds, drugs (e.g., compounds approved for human or veterinary use by the U.S. Food and Drug Administration as provided in the Code of Federal Regulations), vaccines, immunological agents, agents useful in bioprocessing, and mixtures thereof. The targeting agents are described in more detail herein. In certain embodiments, the agents are nutraceutical agents. In certain embodiments, the agents are pharmaceutical agents (e.g., a therapeutic or prophylactic agent). In certain embodiments, the agent is an antibiotic agent (e.g., an anti-bacterial, anti-viral, or anti-fungal agent), anesthetic, steroidal agent, anti-proliferative agent, anti-inflammatory agent, anti-angiogenesis agent, anti-neoplastic agent, anti-cancer agent, anti-diabetic agent, antigen, vaccine, antibody, decongestant, antihypertensive, sedative, birth control agent, progestational agent, anti-cholinergic, analgesic, immunosuppressant, anti-depressant, anti-psychotic, β-adrenergic blocking agent, diuretic, cardiovascular active agent, vasoactive agent, non-steroidal, nutritional agent, anti-allergic agent, or pain-relieving agent. Vaccines may comprise isolated proteins or peptides, inactivated organisms and viruses, dead organisms and viruses, genetically altered organisms or viruses, polynucleotide (e.g., mRNA), and cell extracts. Therapeutic and prophylactic agents may be combined with interleukins, interferon, cytokines, and adjuvants such as cholera toxin, alum, and Freund's adjuvant, etc.

In certain embodiments, an agent to be delivered or used in a composition described herein is a polynucleotide. In certain embodiments, the agent is plasmid DNA (pDNA). In certain embodiments, the agent is single-stranded DNA (ssDNA), double-stranded DNA (dsDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA. In certain embodiments, the agent is RNA. In certain embodiments, the agent is small interfering RNA (siRNA). In certain embodiments, the agent is messenger RNA (mRNA). In certain embodiments, the agent is single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the agent is an RNA that carries out RNA interference (RNAi). The phenomenon of RNAi is discussed in greater detail, for example, in the following references: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, upon delivery of an RNA into a subject, tissue, or cell, the RNA is able to interfere with the expression of a specific gene in the subject, tissue, or cell. In certain embodiments, the agent is a pDNA, siRNA, mRNA, or a combination thereof.

In some embodiments, the RNA is coding RNA or non-coding RNA. In some embodiments, the coding RNA is messenger RNA (mRNA). In some embodiments, the RNA is precursor messenger RNA. In some embodiments, the non-coding RNA is double-stranded RNA, short hairpin RNA, microRNA, guide RNA, transfer RNA, antisense RNA, long non-coding RNA, signal recognition particle RNA, small cytoplasmic RNA, small nuclear RNA, ribosomal RNA, Piwi-interacting RNA, small nucleolar RNA, or spliced leader RNA. In some embodiments, the non-coding RNA is small interfering RNA. In some embodiments, the RNA is single-stranded RNA, heterogeneous nuclear RNA, satellite RNA, viral RNA, or viral satellite RNA. In some embodiments, the agent is ribozyme or flexizyme. In some embodiments, the polynucleotide is a DNA. In some embodiments, the DNA is a plasmid DNA (pDNA).

In some embodiments, the RNA is an mRNA for inducing functional protein expression. In some embodiments, the RNA is an mRNA that encodes a luciferase. In some embodiments, the RNA is an mRNA that encodes one or more antigens. In some embodiments, the RNA is an mRNA that encodes a chimeric antigen receptor (CAR). In some embodiments, the antigen selected from a viral antigen, a bacterial antigen, a fungal antigen, a parasitic antigen, an influenza antigen, a tumor-associated antigen, a tumor-specific antigen, or any combination thereof. In some embodiments, the antigen comprises a tumor-specific antigen or tumor-associated antigen. In some embodiments, the antigen induces an adaptive immune response. In some embodiments, the RNA is an mRNA that induces expression of a CAR. In some embodiments, the RNA is an mRNA that induces transient expression of a CAR.

In certain embodiments, the polynucleotide may be provided as an antisense agent or RNAi. See, e.g., Fire et al., Nature 391:806-811, 1998. Antisense therapy is meant to include, e.g., administration or in situ provision of single- or double-stranded polynucleotides, or derivatives thereof, which specifically hybridize, e.g., bind, under cellular conditions, with cellular mRNA and/or genomic DNA, or mutants thereof, so as to inhibit the expression of the encoded protein, e.g., by inhibiting transcription and/or translation. See, e.g., Crooke, “Molecular mechanisms of action of antisense drugs,” Biochim. Biophys. Acta 1489(1):31-44, 1999; Crooke, “Evaluating the mechanism of action of anti-proliferative antisense drugs,” Antisense Nucleic Acid Drug Dev. 10(2):123-126, discussion 127, 2000; Methods in Enzymology volumes 313-314, 1999. The binding may be by conventional base pair complementarity, or, for example, in the case of binding to DNA duplexes, through specific interactions in the major groove of the double helix (i.e., triple helix formation). See, e.g., Chan et al., J. Mol. Med. 75(4):267-282, 1997.

In some embodiments, pDNA, siRNA, dsRNA, shRNA, miRNA, mRNA, tRNA, asRNA, and/or RNAi can be designed and/or predicted using one or more of a large number of available algorithms. To give but a few examples, the following resources can be utilized to design and/or predict polynucleotides: algorithms found at Alnylum Online; Dharmacon Online; OligoEngine Online; Molecula Online; Ambion Online; BioPredsi Online; RNAi Web Online; Chang Bioscience Online; Invitrogen Online; LentiWeb Online GenScript Online; Protocol Online; Reynolds et al., 2004, Nat. Biotechnol., 22:326; Naito et al., 2006, Nucleic Acids Res., 34: W448; Li et al., 2007, RNA, 13:1765; Yiu et al., 2005, Bioinformatics, 21:144; and Jia et al., 2006, BMC Bioinformatics, 7: 271.

The polynucleotide included in a composition may be of any size or sequence, and they may be single- or double-stranded. In certain embodiments, the polynucleotide includes at least about 30, at least about 100, at least about 300, at least about 1,000, at least about 3,000, or at least about 10,000 base pairs. In certain embodiments, the polynucleotide includes less than about 10,000, less than about 3,000, less than about 1,000, less than about 300, less than about 100, or less than about 30 base pairs. Combinations of the above ranges (e.g., at least about 100 and less than about 1,000) are also within the scope of the invention. The polynucleotide may be provided by any means known in the art. In certain embodiments, the polynucleotide is engineered using recombinant techniques. See, e.g., Ausubel et al., Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989). The polynucleotide may also be obtained from natural sources and purified from contaminating components found normally in nature. The polynucleotide may also be chemically synthesized in a laboratory. In certain embodiments, the polynucleotide is synthesized using standard solid phase chemistry. The polynucleotide may be isolated and/or purified. In certain embodiments, the polynucleotide is substantially free of impurities. In certain embodiments, the polynucleotide is at least about 50%, at least about 60%, at least about 70%, at least about 80%, at least about 90%, at least about 95%, or at least about 99% free of impurities.

The polynucleotide may be modified by physical, chemical, and/or biological means. The modifications include methylation, phosphorylation, and end-capping, etc. In certain embodiments, the modifications lead to increased stability of the polynucleotide.

Wherever a polynucleotide is employed in the composition, a derivative of the polynucleotide may also be used. These derivatives include products resulted from modifications of the polynucleotide in the base moieties, sugar moieties, and/or phosphate moieties of the polynucleotide. Modified base moieties include, but are not limited to, 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine. Modified sugar moieties include, but are not limited to, 2′-fluororibose, ribose, 2′-deoxyribose, 3′-azido-2′,3′-dideoxyribose, 2′,3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′-N-phosphoramidite linkages. Combinations of the various modifications may be used in a single polynucleotide. These modified polynucleotides may be provided by any means known in the art; however, as will be appreciated by those of skill in the art, the modified polynucleotides may be prepared using synthetic chemistry in vitro.

The polynucleotide described herein may be in any form, such as a circular plasmid, a linearized plasmid, a cosmid, a viral genome, a modified viral genome, and an artificial chromosome.

The polynucleotide described herein may be of any sequence. In certain embodiments, the polynucleotide encodes a protein or peptide. The encoded protein may be an enzyme, structural protein, receptor, soluble receptor, ion channel, active (e.g., pharmaceutically active) protein, cytokine, interleukin, antibody, antibody fragment, antigen, coagulation factor, albumin, growth factor, hormone, and insulin, etc. The polynucleotide may also comprise regulatory regions to control the expression of a gene. These regulatory regions may include, but are not limited to, promoters, enhancer elements, repressor elements, TATA boxes, ribosomal binding sites, and stop sites for transcription, etc. In certain embodiments, the polynucleotide is not intended to encode a protein. For example, the polynucleotide may be used to fix an error in the genome of the cell being transfected.

In certain embodiments, the polynucleotide is immunomodulatory, e.g., immunostimulatory, or immunosuppressive.

In certain embodiments, the polynucleotide described herein comprises a sequence encoding an antigenic peptide or protein. A composition containing the polynucleotide can be delivered to a subject to induce an immunologic response sufficient to decrease the chance of a subsequent infection and/or lessen the symptoms associated with such an infection. The polynucleotide of these vaccines may be combined with interleukins, interferon, cytokines, and/or adjuvants described herein.

The antigenic protein or peptides encoded by the polynucleotide may be derived from bacterial organisms, such as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, and Camphylobacter jejuni; from viruses, such as smallpox virus, influenza A virus, influenza B virus, respiratory syncytial virus, parainfluenza virus, measles virus, HIV virus, varicella-zoster virus, herpes simplex 1 virus, herpes simplex 2 virus, cytomegalovirus, Epstein-Barr virus, rotavirus, rhinovirus, adenovirus, papillomavirus, poliovirus, mumps virus, rabies virus, rubella virus, coxsackieviruses, equine encephalitis virus, Japanese encephalitis virus, yellow fever virus, Rift Valley fever virus, hepatitis A virus, hepatitis B virus, hepatitis C virus, hepatitis D virus, and hepatitis E virus; and from fungal, protozoan, or parasitic organisms, such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, and Schistosoma mansoni.

In some embodiments, the antigenic protein or peptides encoded by the polynucleotide may be derived from coronavirus (e.g., SARS-CoV-2), influenza A virus, or influenza B virus. In some embodiments, the antigenic protein or peptides encoded by the polynucleotide may be derived from SARS-CoV-2 (e.g., SARS-CoV-2 B.1.617.2). In some embodiments, the antigenic protein or peptides encoded by the polynucleotide may be derived from SARS-CoV-2 B.1.617.2. In some embodiments, the antigenic protein or peptides encoded by the polynucleotide may be derived from influenza A virus (e.g., hemagglutinin). In some embodiments, the antigenic protein or peptides encoded by the polynucleotide may be derived from influenza B virus.

In some embodiments, the agent is an mRNA that encodes for the SARS-CoV-2 B.1.617.2 spike protein. In some embodiments, the agent is an mRNA that encodes for the SARS-CoV-2 B.1.617.2 spike protein with 984P and 985P mutations. In some embodiments, the agent is an mRNA that encodes for a hemagglutinin protein of influenza A virus. In some embodiments, the agent is an mRNA that encodes for the H3 protein of influenza A virus.

In certain embodiments, the agent is erythropoietin (EPO), e.g., recombinant human erythropoietin (rhEPO). Erythropoietin is an essential hormone for red blood cell production, and may be used in treating hematological diseases, e.g., anemia., such as anemia resulting from chronic kidney disease, chemotherapy induced anemia in patients with cancer, inflammatory bowel disease (Crohn's disease and ulcerative colitis) and myelodysplasia from the treatment of cancer (chemotherapy and radiation). Recombinant human erythropoietins available for use include EPOGEN/PROCRIT (Epoetin alfa, rINN) and ARANESP (Darbepoetin alfa, rINN).

An agent described herein may be non-covalently (e.g., complexed or encapsulated) attached to a compound as described herein, or included in a composition described herein. In certain embodiments, upon delivery of the agent into a cell, the agent is able to interfere with the expression of a specific gene in the cell.

In certain embodiments, the agent in a composition that is delivered to a subject in need thereof may be a mixture of two or more agents that may be useful as, e.g., combination therapies. The compositions including the two or more agents can be administered to achieve a synergistic effect. In certain embodiments, the compositions including the two or more agents can be administered to improve the activity and/or bioavailability, reduce and/or modify the metabolism, inhibit the excretion, and/or modify the distribution within the body of a subject, of each one of the two or more agents. It will also be appreciated that the therapy employed may achieve a desired effect for the same disorder, and/or it may achieve different effects.

The compositions (e.g., pharmaceutical compositions) can be administered concurrently with, prior to, or subsequent to the one or more agents (e.g., pharmaceutical agents). The two or more agents may be useful for treating and/or preventing a same disease or different diseases described herein. Each one of the agents may be administered at a dose and/or on a time schedule determined for that agent. The agents may also be administered together with each other and/or with the composition described herein in a single dose or administered separately in different doses. The particular combination to employ in a regimen will take into account compatibility of the agents and/or the desired therapeutic and/or prophylactic effect to be achieved. In general, it is expected that the agents utilized in combination be utilized at levels that do not exceed the levels at which they are utilized individually. In some embodiments, the levels utilized in combination will be lower than those utilized individually.

### Targeting Agents

Since it is often desirable to target a particular cell, collection of cells, or tissue, compounds provided herein, and the complexes, liposomes, micelles, and particles (e.g., microparticles and nanoparticles) thereof, may be modified to include targeting moieties. For example, a compound provided herein may include a targeting moiety. A variety of agents or regions that target particular cells are known in the art. See, e.g., Cotten et al., Methods Enzym. 217:618, 1993. The targeting agent may be included throughout a particle of a compound provided herein or may be only on the surface of the particle. The targeting agent may be a protein, peptide, carbohydrate, glycoprotein, lipid, small molecule, or polynucleotide, etc. The targeting agent may be used to target specific cells or tissues or may be used to promote endocytosis or phagocytosis of the particle. Examples of targeting agents include, but are not limited to, antibodies, fragments of antibodies, proteins, peptides, carbohydrates, receptor ligands, sialic acid, and aptamers, etc. If the targeting agent is included throughout a particle, the targeting agent may be included in the mixture that is used to form the particle. If the targeting agent is only on the surface of a particle, the targeting agent may be associated with (e.g., by covalent or non-covalent (e.g., electrostatic, hydrophobic, hydrogen bonding, van der Waals, xT-n stacking) interactions) the formed particle using standard chemical techniques.

### Particles

In some embodiments, a composition including a compound provided herein and an agent is in the form of a particle. In certain embodiments, the compound provided herein and agent form a complex, and the complex is in the form of a particle. In certain embodiments, the compound provided herein encapsulates the agent and is in the form of a particle. In certain embodiments, the compound provided herein is mixed with the agent, and the mixture is in the form of a particle. In some embodiments, the particle encapsulates the agent.

In certain embodiments, a complex of a compound provided herein and an agent in a composition of is in the form of a particle. In some embodiments, the particle is a nanoparticle or a microparticle. In certain embodiments, the particle is a microparticle (i.e., particle having a characteristic dimension of less than about 1 millimeter and at least about 1 micrometer, where the characteristic dimension of the particle is the smallest cross-sectional dimension of the particle). In certain embodiments, the particle is a nanoparticle (i.e., a particle having a characteristic dimension of less than about 1 micrometer and at least about 1 nanometer, where the characteristic dimension of the particle is the smallest cross-sectional dimension of the particle). In certain embodiments, the average diameter of the particle is at least about 10 nm, at least about 30 nm, at least about 100 nm, at least about 300 nm, at least about 1 μm, at least about 3 μm, at least about 10 μm, at least about 30 μm, at least about 100 μm, at least about 300 μm, or at least about 1 mm. In certain embodiments, the average diameter of the particle is less than about 1 mm, less than about 300 μm, less than about 100 μm, less than about 30 μm less than about 10 μm, less than about 3 μm, less than about 1 μm, less than about 300 nm, less than about 100 nm, less than about 30 nm, or less than about 10 nm. Combinations of the above ranges (e.g., at least about 100 nm and less than about 1 μm) are also within the scope of the present invention.

The particles described herein may include additional materials such as polymers (e.g., synthetic polymers (e.g., PEG, PLGA) and natural polymers (e.g., phospholipids)). In certain embodiments, the additional materials are approved by a regulatory agency, such as the U.S. FDA, for human and veterinary use.

The particles may be prepared using any method known in the art, such as precipitation, milling, spray drying, single and double emulsion solvent evaporation, solvent extraction, phase separation, and simple and complex coacervation. In certain embodiments, methods of preparing the particles are the double emulsion process and spray drying. The conditions used in preparing the particles may be altered to yield particles of a desired size or property (e.g., hydrophobicity, hydrophilicity, external morphology, “stickiness”, shape, polydispersity, etc.). The method of preparing the particle and the conditions (e.g., solvent, temperature, concentration, and air flow rate, etc.) used may also depend on the agent being complexed, encapsulated, or mixed, and/or the composition of the matrix.

Methods developed for making particles for delivery of agents that are included in the particles are described in the literature. See, e.g., Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al., Reactive Polymers 6:275-283, 1987; Mathiowitz et al., J. Appl. Polymer Sci. 35:755-774, 1988.

If the particles prepared by any of the above methods have a size range outside of the desired range, the particles can be sized, for example, using a sieve. The particles may also be coated. In certain embodiments, the particles are coated with a targeting agent. In certain embodiments, the particles are coated with a surface-altering agent. In some embodiments, the particles are coated to achieve desirable surface properties (e.g., a particular charge).

In certain embodiments, the polydispersity index (PDI, determined by dynamic light scattering) of the particles described herein (e.g., particles included in a composition described herein) is between 0.01 and 0.9, between 0.1 and 0.9, between 0.1 and 0.7, between 0.1 and 0.5, between 0.01 and 0.4, between 0.03 and 0.4, between 0.1 and 0.4, between 0.01 and 0.3, between 0.03 and 0.3, or between 0.1 and 0.3.

### Micelles and Liposomes

A composition including one or more compounds provided herein and an agent may be in the form of a micelle, liposome, or lipoplex. In certain embodiments, the compound provided herein is in the form of a micelle or liposome. In certain embodiments, the agent is in the form of a micelle or liposome. In certain embodiments, the compound provided herein and agent form a complex, and the complex is in the form of a micelle or liposome. In certain embodiments, the compound provided herein encapsulates the agent and is in the form of a micelle or liposome. In certain embodiments, the compound provided herein is mixed with the agent, and the mixture is in the form of a micelle or liposome. Micelles and liposomes are particularly useful in delivering an agent, such as a hydrophobic agent. When the micelle or liposome is complexed with (e.g., encapsulates or covers) a polynucleotide, the resulting complex may be referred to as a “lipoplex.” Many techniques for preparing micelles and liposomes are known in the art, and any such method may be used herein to make micelles and liposomes.

In certain embodiments, liposomes are formed through spontaneous assembly. In some embodiments, liposomes are formed when thin lipid films or lipid cakes are hydrated and stacks of lipid crystalline bilayers become fluid and swell. The hydrated lipid sheets detach during agitation and self-close to form large, multilamellar vesicles (LMV). This prevents interaction of water with the hydrocarbon core of the bilayers at the edges. Once these liposomes have formed, reducing the size of the liposomes can be modified through input of sonic energy (sonication) or mechanical energy (extrusion). See, e.g., Walde, P. “Preparation of Vesicles (Liposomes)” In Encylopedia of Nanoscience and Nanotechnology; Nalwa, H. S. Ed. American Scientific Publishers: Los Angeles, 2004; Vol. 9, pp. 43-79; Szoka et al., “Comparative Properties and Methods of Preparation of Lipid Vesicles (Liposomes)” Ann. Rev. Biophys. Bioeng. 9:467-508, 1980; each of which is incorporated herein by reference. The preparation of lipsomes may involve preparing a compound provided herein for hydration, hydrating the compound with agitation, and sizing the vesicles to achieve a homogenous distribution of liposomes. A compound provided herein may be first dissolved in an organic solvent in a container to result in a homogeneous mixture. The organic solvent is then removed to form a polymer-derived film. This polymer-derived film is thoroughly dried to remove residual organic solvent by placing the container on a vacuum pump for a period of time. Hydration of the polymer-derived film is accomplished by adding an aqueous medium and agitating the mixture. Disruption of LMV suspensions using sonic energy typically produces small unilamellar vesicles (SUV) with diameters in the range of 15-50 nm. Lipid extrusion is a technique in which a lipid/polymer suspension is forced through a polycarbonate filter with a defined pore size to yield particles having a diameter near the pore size of the filter used. Extrusion through filters with 100 nm pores typically yields large, unilamellar polymer-derived vesicles (LUV) with a mean diameter of 120-140 nm. In certain embodiments, the amount of a compound provided herein in the liposome ranges from about 30 mol % to about 80 mol %, from about 40 mol % to about 70 mol %, or from about 60 mol % to about 70 mol %. In certain embodiments, the compound provided herein employed further complexes an agent, such as a polynucleotide. In such embodiments, the application of the liposome is the delivery of the polynucleotide.

The following scientific papers described other methods for preparing liposomes and micelles: Narang et al., “Cationic Lipids with Increased DNA Binding Affinity for Nonviral Gene Transfer in Dividing and Nondividing Cells,” Bioconjugate Chem. 16:156-68, 2005; Hofland et al., “Formation of stable cationic lipid/DNA complexes for gene transfer,” Proc. Natd. Acad. Sci. USA 93:7305-7309, July 1996; Byk et al., “Synthesis, Activity, and Structure—Activity Relationship Studies of Novel Cationic Lipids for DNA Transfer,” J. Med. Chem. 41(2):224-235, 1998; Wu et al., “Cationic Lipid Polymerization as a Novel Approach for Constructing New DNA Delivery Agents,” Bioconjugate Chem. 12:251-57, 2001; Lukyanov et al., “Micelles from lipid derivatives of water-soluble polymers as delivery systems for poorly soluble drugs,” Advanced Drug Delivery Reviews 56:1273-1289, 2004; Tranchant et al., “Physicochemical optimisation of plasmid delivery by cationic lipids,” J. Gene Med. 6: S24-S35, 2004; van Balen et al., “Liposome/Water Lipophilicity: Methods, Information Content, and Pharmaceutical Applications,” Medicinal Research Rev. 24(3):299-324, 2004.

### Kits

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for preventing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for reducing the risk of developing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for delivering an agent to a subject or cell. In certain embodiments, the kits are useful for delivering a polynucleotide to a subject or cell. In certain embodiments, the kits are useful for delivering mRNA to a subject or cell. In certain embodiments, the kits are useful for delivering mRNA that encodes a CAR to a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for preventing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for reducing the risk of developing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for delivering an agent to a subject or cell. In certain embodiments, the kits and instructions provide for delivering a polynucleotide to a subject or cell. In certain embodiments, the kits and instructions provide for delivering mRNA to a subject or cell. A kit described herein may include one or more additional pharmaceutical agents described herein as a separate composition.

### Methods of Treatment and Uses

Also provided herein are methods for treating and/or preventing a disease, disorder, or condition in a subject, comprising administering to the subject a composition comprising an agent and a compound of Formula (I), or a pharmaceutically acceptable salt thereof.

In certain embodiments, the disease, disorder, or condition is a genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease. In some embodiments, the disease, disorder, or condition is a genetic disease. In some embodiments, the disease, disorder, or condition is a proliferative disease. In some embodiments, the disease, disorder, or condition is a hematological disease. In some embodiments, the disease, disorder, or condition is a neurological disease. In some embodiments, the disease, disorder, or condition is a liver disease or a spleen disease. In some embodiments, the disease, disorder, or condition is a liver disease. In some embodiments, the disease, disorder, or condition is a spleen disease. In some embodiments, the disease, disorder, or condition is a lung disease. In some embodiments, the disease, disorder, or condition is a painful condition. In some embodiments, the disease, disorder, or condition is a psychiatric disorder. In some embodiments, the disease, disorder, or condition is a musculoskeletal disease. In some embodiments, the disease, disorder, or condition is a metabolic disorder. In some embodiments, the disease, disorder, or condition is an inflammatory disease. In some embodiments, the disease, disorder, or condition is an autoimmune disease.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In some embodiments, the agent is any agent provided herein. In certain embodiments, the agent is a polynucleotide. In some embodiments, the agent is mRNA. In some embodiments, the agent is an mRNA for inducing functional protein expression. In some embodiments, the agent is an mRNA that encodes a CAR. In some embodiments, the agent is an mRNA that induces expression of a CAR. In some embodiments, the agent is an mRNA that induces transient expression of a CAR.

In some embodiments, the agent is an mRNA that encodes for the SARS-CoV-2 B.1.617.2 spike protein. In some embodiments, the agent is an mRNA that encodes for the SARS-CoV-2 B.1.617.2 spike protein with 984P and 985P mutations. In some embodiments, the agent is an mRNA that encodes for a hemagglutinin protein of influenza A virus. In some embodiments, the agent is an mRNA that encodes for the H3 protein of influenza A virus.

### Additional Methods and Uses

Also provided herein are methods of delivering an agent (e.g., a polynucleotide) to a subject or a cell, comprising administering to the subject or contacting the cell with a composition comprising an agent and a compound provided herein (e.g., a compound of Formula (I)), or a pharmaceutically acceptable salt thereof.

In some embodiments, the agent is any agent provided herein. In some embodiments, the agent is an organic molecule, inorganic molecule, polynucleotide, protein, peptide, polynucleotide, targeting agent, an isotopically labeled chemical compound, vaccine, an immunological agent, or an agent useful in bioprocessing. In certain embodiments, the agent is a polynucleotide. In some embodiments, the agent is mRNA. In some embodiments, the RNA is coding RNA or non-coding RNA. In some embodiments, the coding RNA is messenger RNA (mRNA). In some embodiments, the agent is an mRNA for inducing functional protein expression. In some embodiments, the agent is an mRNA that encodes a CAR. In some embodiments, the agent is an mRNA that induces expression of a CAR. In some embodiments, the agent is an mRNA that induces transient expression of a CAR. In some embodiments, the agent is an mRNA that encodes for the SARS-CoV-2 B.1.617.2 spike protein. In some embodiments, the agent is an mRNA that encodes for the SARS-CoV-2 B.1.617.2 spike protein with 984P and 985P mutations. In some embodiments, the agent is an mRNA that encodes for a hemagglutinin protein of influenza A virus. In some embodiments, the agent is an mRNA that encodes for the H3 protein of influenza A virus. In some embodiments, the RNA is precursor messenger RNA. In some embodiments, the non-coding RNA is double-stranded RNA, short hairpin RNA, microRNA, guide RNA, transfer RNA, antisense RNA, long non-coding RNA, signal recognition particle RNA, small cytoplasmic RNA, small nuclear RNA, ribosomal RNA, Piwi-interacting RNA, small nucleolar RNA, or spliced leader RNA. In some embodiments, the non-coding RNA is small interfering RNA. In some embodiments, the agent is ribozyme or flexizyme. In some embodiments, the RNA is single-stranded RNA, heterogeneous nuclear RNA, satellite RNA, viral RNA, or viral satellite RNA. In some embodiments, the polynucleotide is a DNA. In some embodiments, the DNA is a plasmid DNA (pDNA). In some embodiments, the polynucleotide is delivered to the liver or spleen of the subject. In some embodiments, the composition is administered intravenously.

In some embodiments, the agent is delivered to a subject. In some embodiments, the agent is delivered to the liver or spleen of the subject. In some embodiments, the agent is delivered to the liver of the subject. In certain embodiments, the agent is delivered to the spleen of the subject. In some embodiments, the polynucleotide is delivered to a subject. In some embodiments, the polynucleotide is delivered to the liver or spleen of the subject. In some embodiments, the polynucleotide is delivered to the liver of the subject. In certain embodiments, the polynucleotide is delivered to the spleen of the subject. In some embodiments, the mRNA is delivered to a subject. In some embodiments, the mRNA is delivered to the liver or spleen of the subject. In some embodiments, the mRNA is delivered to the liver of the subject. In certain embodiments, the mRNA is delivered to the spleen of the subject.

In certain embodiments, the agent is delivered to a cell. In certain embodiments, the polynucleotide is delivered to a cell. In certain embodiments, the mRNA is delivered to a cell. In some embodiments, the cell is in vivo, e.g., in an organism. In certain embodiments, the cell is in vitro, e.g., in cell culture. In some embodiments, the cell is ex vivo, meaning the cell is removed from an organism prior to the delivery. In some embodiments, the cell is ex vivo and is readministered to the subject.

In some embodiments, the cell is an immune cell, T cell, resident T cells, B cell, natural killer (NK) cell, cancerous cell, cell associated with a disease or disorder, epithelial cell, endothelial cell, or tumor cell. In some embodiments, the cell is a cell in tissue associated with a disease or disorder, brain tissue, central nervous system tissue, pulmonary tissue, apical surface tissue, liver tissue, intestine tissue, colon tissue, small intestine tissue, large intestine tissue, feces, bone marrow, macrophages, spleen tissue, muscles tissue, joint tissue, diseased tissues, lymph node tissue, lymphatic circulation, or any combination thereof. In some embodiments, the cell is a hematopoietic stem cell or an immune cell. In some embodiments, the cell is a Hela cell or a Jurkat cell.

In some embodiments, the composition is administered by any method provided herein. In certain embodiments, the composition is administered intravenously or intramuscularly. In certain embodiments, the composition is administered intravenously. In certain embodiments, the composition is administered intramuscularly.

In another aspect, provided herein is a method of preparing a compound of Formula (II):

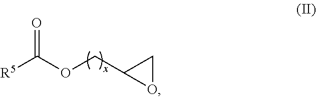

the method comprising reacting a compound of Formula (III):

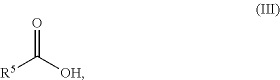

or a salt thereof, with a carbodiimide coupling agent, or a salt thereof, and a compound of Formula (IV):

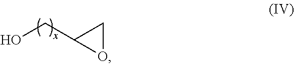

or a salt thereof, wherein:
x is an integer from 1 to 10, inclusive; and
R⁵ is optionally substituted C₄-C₂₅ saturated or unsaturated aliphatic or optionally substituted C₄-C₂₅ saturated or unsaturated heteroaliphatic.

In another aspect, provided herein is a method of preparing a compound of Formula (I):

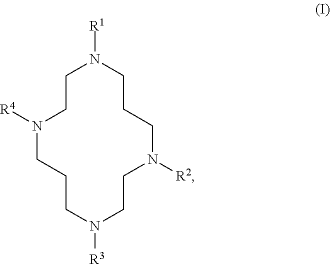

or a salt thereof, the method comprising reacting a compound of Formula (II):

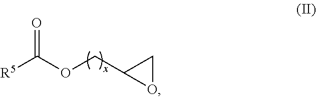

with a compound of Formula (V): or a salt thereof, wherein:

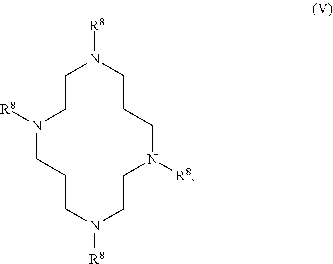

each of R¹, R², R³, and R⁴ is independently hydrogen, a nitrogen protecting group, or

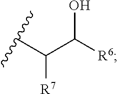

one of R⁶ and R⁷ is hydrogen, and the other is

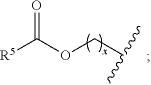

each instance of R⁵ is independently optionally substituted C₄-C₂₅ saturated or unsaturated aliphatic or optionally substituted C₄-C₂₅ saturated or unsaturated heteroaliphatic; and
each x independently is an integer from 1 to 10, inclusive;
provided that at least one of R¹, R², R³, and R⁴ is

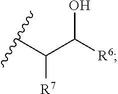

each instance of R⁸ is independently hydrogen or a nitrogen protecting group.

In some embodiments, an excess of the compound of Formula (II) is used. In some embodiments, the excess of the compound of Formula (II) is one equivalent in excess of the number of reactive amines per compound of Formula (V), or a salt thereof. In some embodiments, the excess of the compound of Formula (II) is five equivalents relative to the compound of Formula (V), or a salt thereof.

In some embodiments, the reaction is performed under microwave radiation.

In some embodiments, the reaction is performed in a polar solvent. In some embodiments, the reaction is performed in a polar protic solvent. In some embodiments, the reaction is performed in an alcohol. In some embodiments, the reaction is performed in methanol, ethanol, n-propanol, or isopropanol. In some embodiments, the reaction is performed in isopropanol.

In some embodiments, the reaction is performed at about 50-130° C. In some embodiments, the reaction is performed at least 50° C. In some embodiments, the reaction is performed at least 60° C. In some embodiments, the reaction is performed at least 70° C. In some embodiments, the reaction is performed at least 80° C. In some embodiments, the reaction is performed at least 85° C. In some embodiments, the reaction is performed at about 80-100° C. In some embodiments, the reaction is performed at about 85-95° C. In some embodiments, the reaction is performed at about 90° C.

In some embodiments, the method further comprises a purification step. In some embodiments, the method further comprises chromatographic purification. In some embodiments, the chromatographic purification step comprises silica gel column chromatography.

In some embodiments, the method further comprises an isolation step. In some embodiments, the method further comprises a step of isolating the compound of Formula (I), or a salt thereof. In some embodiments, the method further comprises a step of isolating the compound of Formula (II).

In some embodiments, the method further comprises a deprotection step. In some embodiments, the deprotection step is performed as an initial step. In some embodiments, the deprotection step is performed simultaneously to the reaction.

In some embodiments, the carbodiimide coupling agent is 1-ethyl-3-(3-dimethylaminopropyl)carbodiimide, dicyclohexylcarbodiimide, or diisopropylcarbodiimide, or a salt thereof. In some embodiments, the carbodiimide coupling agent is 1-ethyl-3-(3-dimethylaminopropyl)carbodiimide, dicyclohexylcarbodiimide, or diisopropylcarbodiimide, or a hydrochloride salt thereof. In some embodiments, the carbodiimide coupling agent is 1-ethyl-3-(3-dimethylaminopropyl)carbodiimide, dicyclohexylcarbodiimide, or diisopropylcarbodiimide, or a hydroiodide salt thereof. In some embodiments, the carbodiimide coupling agent is 1-ethyl-3-(3-dimethylaminopropyl)carbodiimide, or a salt thereof. In some embodiments, the carbodiimide coupling agent is 1-ethyl-3-(3-dimethylaminopropyl)carbodiimide hydrochloride. In some embodiments, the carbodiimide coupling agent is dicyclohexylcarbodiimide, or a salt thereof. In some embodiments, the carbodiimide coupling agent is diisopropylcarbodiimide, or a salt thereof.

As defined herein, each instance of R⁸ is independently hydrogen or a nitrogen protecting group. In some embodiments, at least one instance of R⁸ is hydrogen, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C-20 alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. In some embodiments, at least one instance of R⁸ is hydrogen, a carbamate group, or a sulfonamide group. In some embodiments, at least one instance of R⁸ is hydrogen, Bn, Boc, Cbz, Fmoc, trifluoroacetyl, triphenylmethyl, acetyl, or Ts. In some embodiments, at least one instance of R⁸ is hydrogen. In some embodiments, at least one instance of R⁸ is a nitrogen protecting group. In some embodiments, at least one instance of R⁸ is —OH, —OR^{aa}, —N(R^{cc})₂, —C(═O)R^{aa}, —C(═O)N(R^{cc})₂, —CO₂R^{aa}, —SO₂R′, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. In some embodiments, at least one instance of R⁸ is a carbamate group or a sulfonamide group. In some embodiments, at least one instance of R⁸ is Bn, Boc, Cbz, Fmoc, trifluoroacetyl, triphenylmethyl, acetyl, or Ts.

As defined herein, R⁵ is optionally substituted C₄-C₂₅ saturated or unsaturated aliphatic or optionally substituted C₄-C₂₅ saturated or unsaturated heteroaliphatic.

In some embodiments, R⁵ is optionally substituted C₄-C₂₅ saturated or unsaturated aliphatic. In some embodiments, R⁵ is optionally substituted C₄-C₁₂ saturated or unsaturated aliphatic. In some embodiments, R⁵ is optionally substituted C₁₀-C₂₀ saturated or unsaturated aliphatic. In some embodiments, R⁵ is optionally substituted C₁₂-C₂₅ saturated or unsaturated aliphatic.

In some embodiments, R⁵ is optionally substituted C₄-C₂₅ alkyl or optionally substituted C₄-C₂₅ alkenyl. In some embodiments, R⁵ is optionally substituted C₄-C₁₀ alkyl or optionally substituted C₄-C₁₀ alkenyl. In some embodiments, R⁵ is optionally substituted C₁₀-C₂₀ alkyl or optionally substituted C₁₀-C₂₀ alkenyl. In some embodiments, R⁵ is optionally substituted C₁₂-C₂₅ alkyl or optionally substituted C₁₂-C₂₅ alkenyl.

In some embodiments, R⁵ is optionally substituted C₄-C₂₅ alkyl or optionally substituted C₄-C₂₅ alkynyl. In some embodiments, R⁵ is optionally substituted C₄-C₁₀ alkyl or optionally substituted C₄-C₁₀ alkynyl. In some embodiments, R⁵ is optionally substituted C₁₀-C₂₀ alkyl or optionally substituted C₁₀-C₂₀ alkynyl. In some embodiments, R⁵ is optionally substituted C₁₂-C₂₅ alkyl or optionally substituted C₁₂-C₂₅ alkynyl.

In some embodiments, R⁵ is optionally substituted C₄-C₂₅ alkynyl or optionally substituted C₄-C₂₅ alkenyl. In some embodiments, R⁵ is optionally substituted C₄-C₁₀ alkynyl or optionally substituted C₄-C₁₀ alkenyl. In some embodiments, R⁵ is optionally substituted C₁₀-C₂₀ alkynyl or optionally substituted C₁₀-C₂₀ alkenyl. In some embodiments, R⁵ is optionally substituted C₁₂-C₂₅ alkynyl or optionally substituted C₁₂-C₂₅ alkenyl.

In some embodiments, R⁵ is branched C₄-C₂₅ alkyl. In some embodiments, R⁵ is branched C₄-C₁₂ alkyl. In some embodiments, R⁵ is branched C₁₀-C₂₀ alkyl. In some embodiments, R⁵ is branched C₁₂-C₂₅ alkyl. In some embodiments, R⁵ is unbranched C₄-C₂₅ alkyl. In some embodiments, R⁵ is unbranched C₄-C₁₂ alkyl. In some embodiments, R⁵ is unbranched C₁₀-C₂₀ alkyl. In some embodiments, R⁵ is unbranched C₁₂-C₂₅ alkyl.

In some embodiments, R⁵ is branched C₄-C₂₅ alkenyl. In some embodiments, R⁵ is branched C₄-C₁₂ alkenyl. In some embodiments, R⁵ is branched C₁₀-C₂₀ alkenyl. In some embodiments, R⁵ is branched C₁₂-C₂₅ alkenyl. In some embodiments, R⁵ is unbranched C₄-C₂₅ alkenyl. In some embodiments, R⁵ is unbranched C₄-C₁₂ alkenyl. In some embodiments, R⁵ is unbranched C₁₀-C₂₀ alkenyl. In some embodiments, R⁵ is unbranched C₁₂-C₂₅ alkenyl.

In some embodiments, R⁵ is unbranched C₁₁ alkenyl. In some embodiments, the C₁₁ alkenyl comprises a single double bond. In some embodiments, the C₁₁ alkenyl comprises double bonds. In some embodiments, the C₁₁ alkenyl comprises three double bonds. In some embodiments, the C₁₁ alkenyl comprises only (Z) double bonds.

In some embodiments, R⁵ is branched C₄-C₂₅ alkynyl. In some embodiments, R⁵ is branched C₄-C₁₂ alkynyl. In some embodiments, R⁵ is branched C₁₀-C₂₀ alkynyl. In some embodiments, R⁵ is branched C₁₂-C₂₅ alkynyl. In some embodiments, R⁵ is unbranched C₄-C₂₅ alkynyl. In some embodiments, R⁵ is unbranched C₄-C₁₂ alkynyl. In some embodiments, R⁵ is unbranched C₁₀-C₂₀ alkynyl. In some embodiments, R⁵ is unbranched C₁₂-C₂₅ alkynyl.

In some embodiments, R⁵ is optionally substituted C₄-C₂₅ saturated or unsaturated heteroaliphatic. In some embodiments, R⁵ is optionally substituted C₄-C₂₅ saturated or unsaturated heteroaliphatic comprising O, N, or S atom. In some embodiments, R⁵ is optionally substituted C₄-C₂₅ saturated or unsaturated heteroaliphatic comprising O or S atom. In some embodiments, R⁵ is optionally substituted C₄-C₁₂ saturated or unsaturated heteroaliphatic. In some embodiments, R⁵ is optionally substituted C₄-C₁₂ saturated or unsaturated heteroaliphatic comprising O, N, or S atom. In some embodiments, R⁵ is optionally substituted C₄-C₁₂ saturated or unsaturated heteroaliphatic comprising O or S atom. In some embodiments, R⁵ is optionally substituted C₁₀-C₂₀ saturated or unsaturated heteroaliphatic. In some embodiments, R⁵ is optionally substituted C₁₀-C₂₀ saturated or unsaturated heteroaliphatic comprising O, N, or S atom. In some embodiments, R⁵ is optionally substituted C₁₀-C₂₀ saturated or unsaturated heteroaliphatic comprising O or S atom. In some embodiments, R⁵ is optionally substituted C₁₂-C₂₅ saturated or unsaturated heteroaliphatic. In some embodiments, R⁵ is optionally substituted C₁₂-C₂₅ saturated or unsaturated heteroaliphatic comprising O, N, or S atom. In some embodiments, R⁵ is optionally substituted C₁₂-C₂₅ saturated or unsaturated heteroaliphatic comprising O or S atom.

In some embodiments, R⁵ is optionally substituted C₄-C₂₅ heteroalkyl or optionally substituted C₄-C₂₅ heteroalkenyl. In some embodiments, R⁵ is optionally substituted C₄-C₁₀ heteroalkyl or optionally substituted C₄-C₁₀ heteroalkenyl. In some embodiments, R⁵ is optionally substituted C₁₀-C₂₀ heteroalkyl or optionally substituted C₁₀-C₂₀ heteroalkenyl. In some embodiments, R⁵ is optionally substituted C₁₂-C₂₅ heteroalkyl or optionally substituted C₁₂-C₂₅ heteroalkenyl.

In some embodiments, R⁵ is optionally substituted C₄-C₂₅ heteroalkyl or optionally substituted C₄-C₂₅ heteroalkynyl. In some embodiments, R⁵ is optionally substituted C₄-C₁₀ heteroalkyl or optionally substituted C₄-C₁₀ heteroalkynyl. In some embodiments, R⁵ is optionally substituted C₁₀-C₂₀ heteroalkyl or optionally substituted C₁₀-C₂₀ heteroalkynyl. In some embodiments, R⁵ is optionally substituted C₁₂-C₂₅ heteroalkyl or optionally substituted C₁₂-C₂₅ heteroalkynyl.

In some embodiments, R⁵ is optionally substituted C₄-C₂₅ heteroalkynyl or optionally substituted C₄-C₂₅ heteroalkenyl. In some embodiments, R⁵ is optionally substituted C₄-C₁₀ heteroalkynyl or optionally substituted C₄-C₁₀ heteroalkenyl. In some embodiments, R⁵ is optionally substituted C₁₀-C₂₀ heteroalkynyl or optionally substituted C₁₀-C₂₀ heteroalkenyl. In some embodiments, R⁵ is optionally substituted C₁₂-C₂₅ heteroalkynyl or optionally substituted C₁₂-C₂₅ heteroalkenyl.

In some embodiments, R⁵ is branched C₄-C₂₅ heteroalkyl. In some embodiments, R⁵ is branched C₄-C₁₂ heteroalkyl. In some embodiments, R⁵ is branched C₁₀-C₂₀ heteroalkyl. In some embodiments, R⁵ is branched C₁₂-C₂₅ heteroalkyl. In some embodiments, R⁵ is unbranched C₄-C₂₅ heteroalkyl. In some embodiments, R⁵ is unbranched C₄-C₁₂ heteroalkyl. In some embodiments, R⁵ is unbranched C₁₀-C₂₀ heteroalkyl. In some embodiments, R⁵ is unbranched C₁₂-C₂₅ heteroalkyl.

In some embodiments, R⁵ is branched C₄-C₂₅ heteroalkenyl. In some embodiments, R⁵ is branched C₄-C₁₂ heteroalkenyl. In some embodiments, R⁵ is branched C₁₀-C₂₀ heteroalkenyl. In some embodiments, R⁵ is branched C₁₂-C₂₅ heteroalkenyl. In some embodiments, R⁵ is unbranched C₄-C₂₅ heteroalkenyl. In some embodiments, R⁵ is unbranched C₄-C₁₂ heteroalkenyl. In some embodiments, R⁵ is unbranched C₁₀-C₂₀ heteroalkenyl. In some embodiments, R⁵ is unbranched C₁₂-C₂₅ heteroalkenyl.

In some embodiments, R⁵ is branched C₄-C₂₅ heteroalkynyl. In some embodiments, R⁵ is branched C₄-C₁₂ heteroalkynyl. In some embodiments, R⁵ is branched C₁₀-C₂₀ heteroalkynyl. In some embodiments, R⁵ is branched C₁₂-C₂₅ heteroalkynyl. In some embodiments, R⁵ is unbranched C₄-C₂₅ heteroalkynyl. In some embodiments, R⁵ is unbranched C₄-C₁₂ heteroalkynyl. In some embodiments, R⁵ is unbranched C₁₀-C₂₀ heteroalkynyl. In some embodiments, R⁵ is unbranched C₁₂-C₂₅ heteroalkynyl.

In some embodiments, R⁵ is selected from the group consisting of:

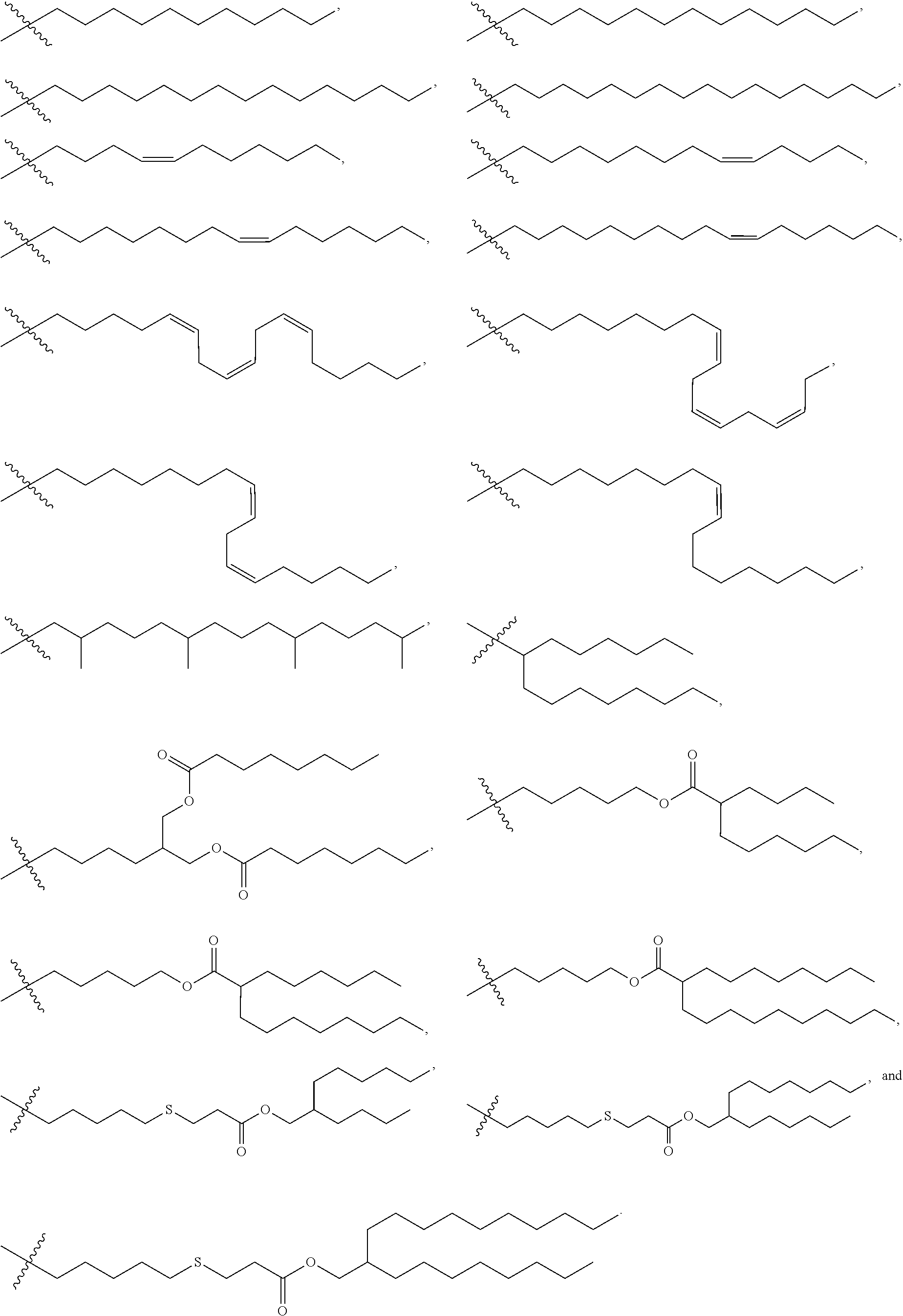

In some embodiments, R⁵ is selected from the group consisting of:

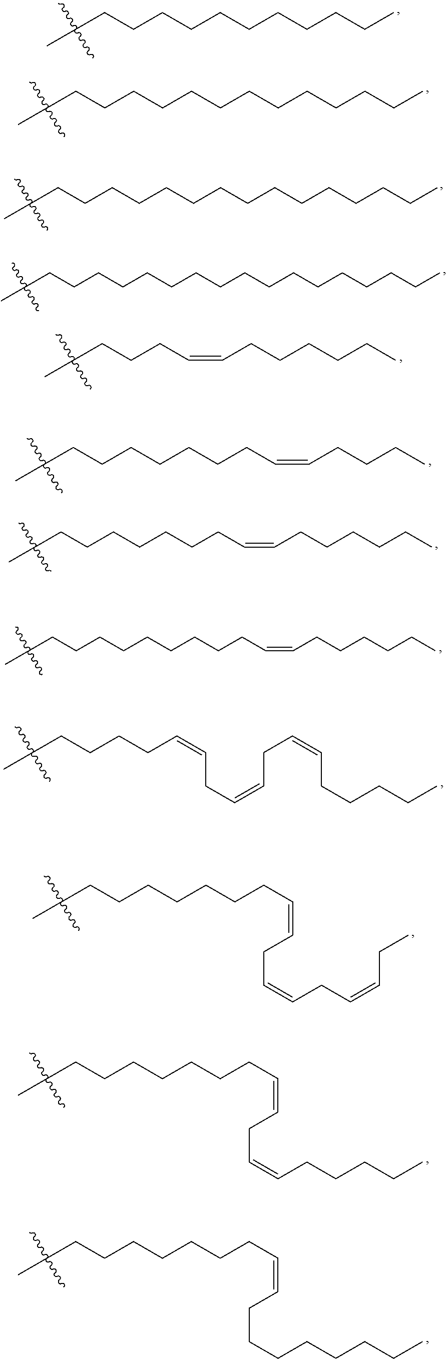

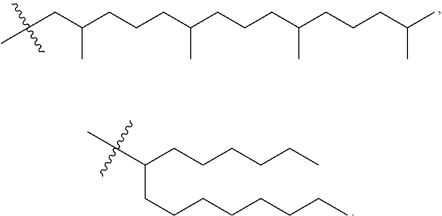 [SMILES: *CC(C)CCCC(C)CCCC(C)CCCC(C)C ; "," ; *C(CCCCCC)CCCCCCCC ; "."]

In some embodiments, R⁵ is selected from the group consisting of:

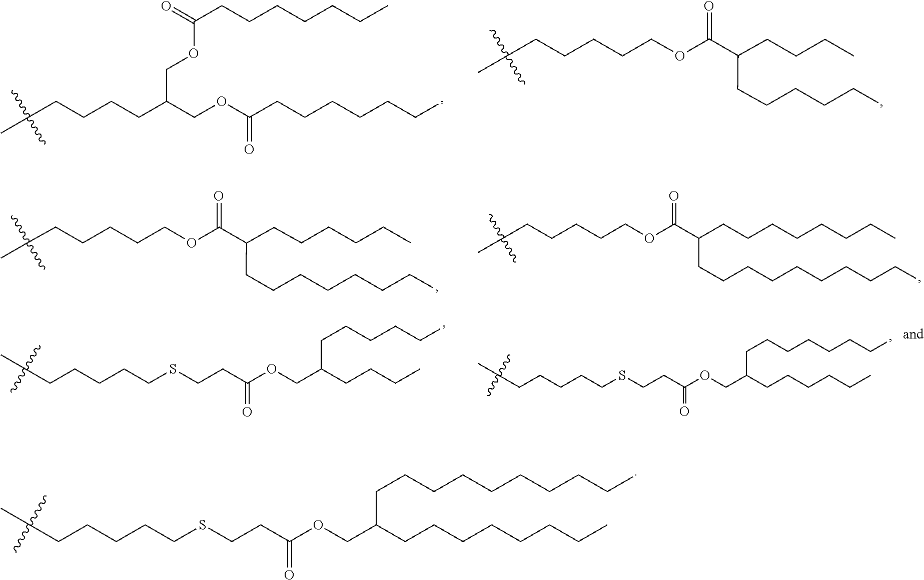

In some embodiments, R⁵ is

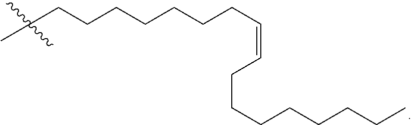 [SMILES: *CCCCCCC/C=C\CCCCCCCC]

In some embodiments, R⁵ is selected from the group consisting of:

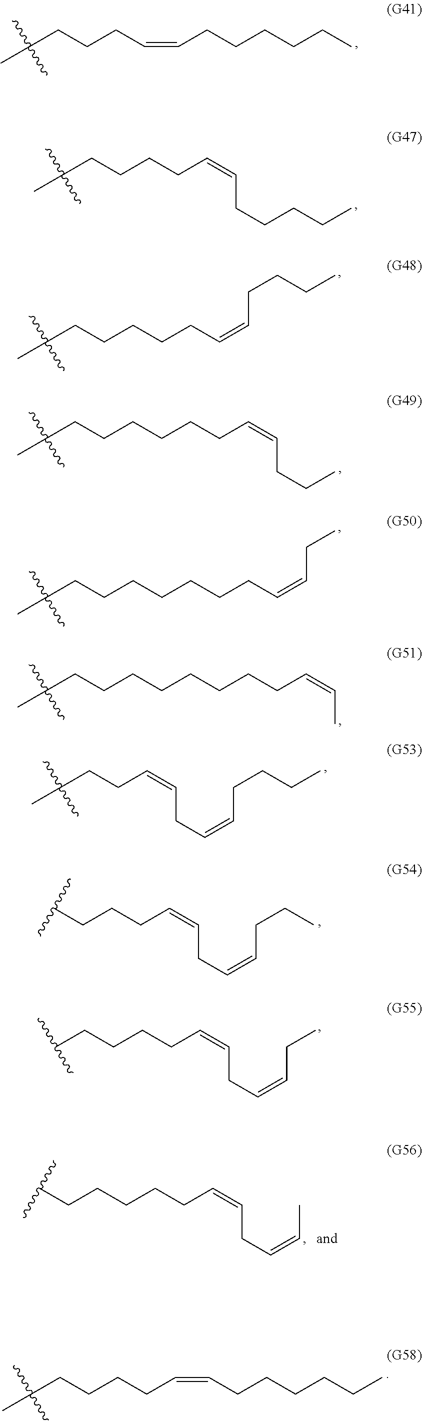

In a particular embodiment, R⁵ is G41. In a particular embodiment, R⁵ is G47. In a particular embodiment, R⁵ is G48. In a particular embodiment, R⁵ is G49. In a particular embodiment, R⁵ is G50. In a particular embodiment, R⁵ is G51. In a particular embodiment, R⁵ is G53. In a particular embodiment, R⁵ is G54. In a particular embodiment, R⁵ is G55. In a particular embodiment, R⁵ is G56.

As defined herein, x is an integer from 1 to 10, inclusive. In some embodiments, x is an integer from 1 to 9, inclusive. In some embodiments, x is an integer from 1 to 8, inclusive. In some embodiments, x is an integer from 1 to 7, inclusive. In some embodiments, x is an integer from 1 to 6, inclusive. In some embodiments, x is an integer from 1 to 5, inclusive. In some embodiments, x is an integer from 1 to 4, inclusive. In some embodiments, x is an integer from 1 to 3, inclusive.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1

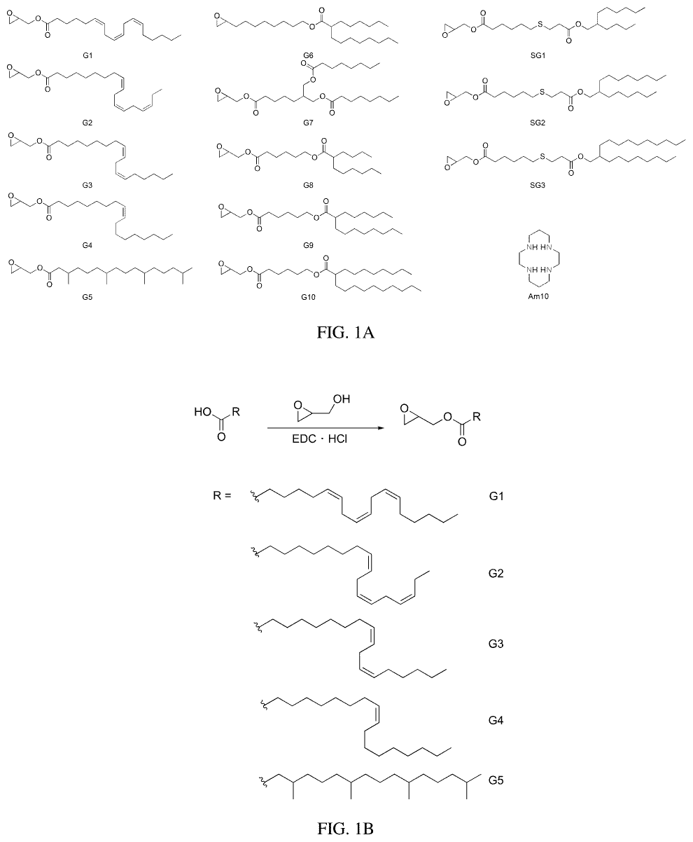

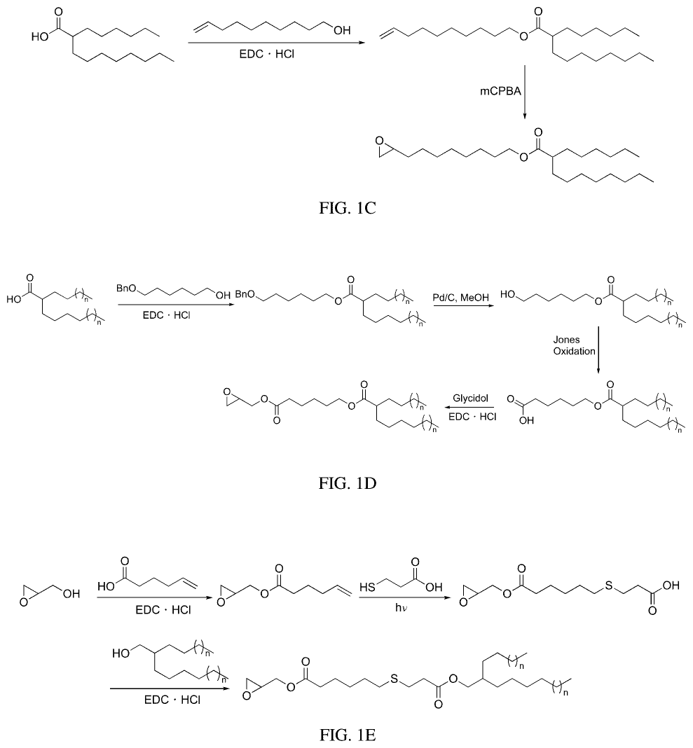

Nucleophilic ring opening of epoxides has been utilized for the synthesis of novel ionizable lipids for RNA delivery.¹ Introducing biodegradable groups such as esters into such lipids may enhance their biosafety for clinical applications. However, labile degradable groups like esters are not compatible with standard reaction conditions. Reaction conditions were developed which enabled syntheses of the biodegradable lipids provided herein using nucleophilic ring opening of epoxides. A cyclic secondary amine was efficient in ring opening of epoxide within fatty acid epoxy esters. Without wishing to be bound by any particular theory, branched lipid tails on these ionizable lipids may enable higher transfection and endosomal escape by forming non-bilayer membrane disrupting conical shape during interactions with the anionic phospholipids.² The lipid tail structures for the first-generation library of ionizable lipids are shown in FIG. 1A.

### Synthesis of Fatty Acid Epoxy Esters

The fatty acid epoxy esters G1-G5 were synthesized by esterifying various corresponding fatty acids with glycidol (FIG. 1B). The G6-G10 esters were synthesized in few steps as shown in FIGS. 1B-1D. The remaining SG1-SG3 esters were synthesized in few steps as shown in FIG. 1E with only one purification step. The number of ester functionalities was varied in G6-G10 to increase diversity in the structures of the branched lipid tails. For example, G6 has one ester group, while G7 has 3, and G8-G10 and SG1-3 have 2 each. Without wishing to be bound by any particular theory, the number of ester groups and tail length impact the transfection ability of the LNPs.

### Synthesis of the 1^{st} Generation of Ionizable Lipids

The head group was reacted with various fatty acid epoxy esters to provide ionizable lipids. These lipids were named AM10, followed by the corresponding epoxy ester number from FIGS. 1A, 4, 9A, and 9B. For example, AMG104 corresponds to the lipid synthesized using G4.

A microwave vial (0.2-0.5 mL) was charged with the epoxy ester (one equivalent in excess of the number of reactive amines) followed by a 0.42 M solution of the headgroup amine in isopropanol. The total reaction volume was made up to 250 μL. The microwave vial was then sealed and heated to 90° C. for 1 hour. Once the reaction was judged complete by LC-MS, the reaction mixture was directly loaded on a silica gel column and purified using dichloromethane (solvent A) and a mixture (solvent B) of dichloromethane, methanol, and ammonium hydroxide (25:7:1). The fractions containing the desired lipid were concentrated and dried under vacuum.

The identities of the lipids were confirmed using LC-MS and characterized by ¹H and ¹³C NMR spectroscopy.

AMG101. ¹H NMR (600 MHz, CDCl₃) δ 5.38 (pt, J=10.6, 6.3 Hz, 24H), 2.94 (t, J=7.7 Hz, 1H), 2.88-2.73 (m, 9H), 2.73-2.50 (m, 2H), 2.47-2.27 (m, J=4.9, 3.9 Hz, 8H), 2.22-1.88 (m, 9H), 1.85-1.51 (m, 17H), 1.50-1.03 (m, 23H), 0.90 (t, J=6.8 Hz, 12H). ¹³C NMR (151 MHz, CDCl₃) δ 173.58, 173.35, 136.04, 130.95, 130.93, 130.48, 129.63, 129.58, 129.50, 128.46, 128.34, 128.27, 128.24, 128.05, 127.72, 127.58, 127.00, 125.71, 72.54, 68.27, 65.96, 65.25, 65.06, 64.89, 64.84, 49.39, 49.37, 44.70, 44.67, 36.84, 34.04, 33.98, 33.95, 31.60, 31.52, 31.51, 29.33, 29.31, 29.29, 29.14, 29.13, 29.08, 27.25, 27.23, 26.91, 26.84, 26.10, 25.64, 25.62, 24.93, 24.73, 24.64, 24.55, 24.53, 24.49, 22.58, 22.56, 14.10, 14.09. HRMS (ESI) m/z [M+H]⁺ calcd for [C₉₄H₁₆₁N₄O₁₂]+1538.2111, obs 1538.2120.

AMG102. ¹H NMR (600 MHz, CDCl₃) δ 5.37 (ddp, J=33.1, 25.1, 7.8 Hz, 24H), 4.43 (dd, J=12.3, 3.1 Hz, 1H), 4.27-3.97 (m, 5H), 3.93-3.77 (m, 1H), 2.84 (dt, J=27.6, 5.6 Hz, 12H), 2.75-2.48 (m, 3H), 2.35 (tq, J=12.2, 6.1, 5.1 Hz, 10H), 2.20-1.98 (m, 13H), 1.43-1.17 (m, 33H), 1.07-0.84 (m, 12H). ¹³C NMR (151 MHz, CDCl₃) δ 173.75, 131.97, 130.24, 128.30, 128.24, 127.75, 127.12, 74.12, 72.81, 65.99, 65.93, 64.78, 49.41, 44.67, 37.29, 34.19, 34.14, 34.12, 34.08, 34.06, 34.03, 30.20, 29.60, 29.58, 29.51, 29.49, 29.32, 29.24, 29.22, 29.14, 29.10, 29.06, 29.03, 29.00, 28.97, 27.22, 27.20, 26.11, 26.00, 25.62, 25.54, 25.52, 25.32, 24.91, 24.89, 24.87, 24.84, 24.82, 24.79, 20.56, 14.30, 9.75. HRMS (ESI) m/z [M+H]⁺ calcd for [C₉₄H₁₆₁N₄O₁₂]⁺ 1538.2111, obs 1538.2119.

AMG103. ¹H NMR (600 MHz, CDCl₃) δ 5.49-5.19 (m, 16H), 4.16-3.96 (m, 7H), 3.96-3.60 (m, 3H), 2.78 (t, J=7.0 Hz, 8H), 2.35 (p, J=7.2 Hz, 9H), 2.15 (dtd, J=26.2, 13.2, 8.5 Hz, 3H), 2.06 (q, J=7.3 Hz, 15H), 1.81-1.67 (m, 5H), 1.62 (q, J=7.3 Hz, 10H), 1.33 (dtt, J=16.2, 10.3, 5.8 Hz, 56H), 0.90 (t, J=6.9 Hz, 12H). ¹³C NMR (151 MHz, CDCl₃) δ 173.81, 173.80, 173.74, 173.73, 130.23, 130.03, 130.01, 128.07, 127.90, 68.66, 66.30, 66.12, 65.98, 65.92, 65.82, 58.28, 57.66, 53.28, 53.12, 51.68, 50.94, 49.40, 34.19, 34.16, 34.14, 34.12, 34.08, 34.03, 31.53, 29.63, 29.35, 29.33, 29.32, 29.24, 29.22, 29.21, 29.14, 29.09, 27.24, 27.21, 25.64, 25.62, 24.91, 24.89, 24.87, 22.59, 22.57, 14.10. HRMS (ESI) m/z [M+H]⁺ calcd for [C₉₄H₁₆₉N₄O₁₂]⁺ 1546.2737, obs 1546.2751.

AMG104. ¹H NMR (600 MHz, CDCl₃) δ 5.35 (p, J=5.2 Hz, 8H), 4.06 (ddp, J=26.2, 22.2, 5.7, 4.8 Hz, 7H), 3.95-3.71 (m, 2H), 3.06 (tq, J=31.9, 10.7, 9.7 Hz, 3H), 2.93-2.48 (m, 3H), 2.47-2.27 (m, 9H), 2.15 (tq, J=20.3, 8.7, 6.7 Hz, 4H), 2.02 (q, J=6.5 Hz, 17H), 1.84-1.68 (m, 3H), 1.63 (p, J=7.4 Hz, 10H), 1.30 (dd, J=20.4, 7.3 Hz, 85H), 0.89 (t, J=6.9 Hz, 12H). ¹³C NMR (151 MHz, CDCl₃) δ 173.83, 173.81, 173.76, 173.74, 130.02, 129.73, 129.71, 66.11, 65.97, 65.91, 65.81, 63.35, 58.27, 53.12, 34.40, 34.19, 34.17, 34.15, 34.13, 34.09, 34.04, 31.95, 31.91, 29.81, 29.79, 29.77, 29.72, 29.68, 29.67, 29.65, 29.53, 29.52, 29.50, 29.49, 29.46, 29.39, 29.33, 29.30, 29.25, 29.22, 29.17, 29.14, 29.10, 27.28, 27.25, 27.23, 27.19, 24.92, 24.90, 24.88, 22.69, 14.20, 14.19, 14.17, 14.16, 14.14. HRMS (ESI) m/z [M+H]⁺ calcd for [C₉₄H₁₇₇N₄O₁₂]⁺ 1554.3363, obs 1554.3359.

AMG105. ¹H NMR (600 MHz, CDCl₃) δ 4.19-3.98 (m, 8H), 3.98-3.77 (m, 3H), 3.73 (q, J=7.1 Hz, 1H), 3.21-2.97 (m, 3H), 2.86-2.50 (m, 3H), 2.47-2.29 (m, 6H), 2.25-2.07 (m, 10H), 2.02-1.89 (m, J=6.6 Hz, 5H), 1.71 (ddt, J=37.8, 23.4, 8.3 Hz, 4H), 1.54 (dp, J=13.3, 6.7 Hz, 5H), 1.39 (dp, J=14.0, 7.7, 7.3 Hz, 10H), 1.34-1.20 (m, 33H), 1.16 (tdd, J=9.7, 7.4, 3.9 Hz, 8H), 1.12-1.01 (m, 20H), 0.94 (dd, J=6.8, 3.3 Hz, 15H), 0.88 (d, J=6.7 Hz, 27H), 0.85 (d, J=6.7 Hz, 32H). ¹³C NMR (151 MHz, CDCl₃) δ 173.31, 173.25, 173.22, 66.21, 66.00, 65.83, 65.74, 58.42, 41.69, 41.67, 39.37, 37.49, 37.46, 37.42, 37.40, 37.35, 37.30, 37.27, 37.18, 37.10, 32.80, 32.78, 32.77, 30.38, 30.35, 30.33, 30.31, 27.98, 27.96, 24.82, 24.81, 24.48, 24.40, 24.38, 22.74, 22.73, 22.65, 22.63, 22.61, 19.80, 19.77, 19.75, 19.73, 19.70, 19.68, 19.62. HRMS (ESI) m/z [M+H]⁺ calcd for [C₁₀₂H₂₀₁N₄O₁₂]⁺ 1674.5241, obs 1674.5241.

AMG106. ¹H NMR (600 MHz, CDCl₃) δ 4.07 (t, J=6.7 Hz, 8H), 3.73-3.45 (m, 4H), 3.07 (dq, J=17.1, 9.3, 7.3 Hz, 1H), 2.32 (tq, J=9.4, 5.3, 4.7 Hz, 6H), 2.17-1.97 (m, 4H), 1.69 (dt, J=19.0, 10.5 Hz, 3H), 1.61 (dp, J=20.8, 6.9 Hz, 18H), 1.52-1.39 (m, 17H), 1.28 (d, J=22.7 Hz, 135H), 0.89 (t, J=7.0 Hz, 24H). ¹³C NMR (151 MHz, CDCl₃) δ 176.74, 70.48, 68.62, 66.79, 64.14, 62.32, 61.99, 61.67, 53.73, 53.54, 53.18, 52.20, 45.87, 35.04, 35.01, 34.90, 34.72, 34.56, 34.51, 32.57, 32.55, 31.91, 31.88, 31.72, 31.70, 29.86, 29.83, 29.79, 29.58, 29.55, 29.53, 29.50, 29.47, 29.43, 29.42, 29.40, 29.39, 29.30, 29.27, 29.24, 29.23, 29.20, 28.73, 27.52, 27.48, 27.44, 25.99, 25.90, 25.88, 25.83, 25.79, 25.77, 25.73, 22.68, 22.61, 22.59, 22.57, 14.17, 14.14, 14.10. HRMS (ESI) m/z [M+H]⁺ calcd for [C₁₁₄H₂₂₅N₄O₁₂]⁺ 1842.7119, obs 1842.7125.

AMG107. ¹H NMR (600 MHz, CDCl₃) δ 4.08 (dd, J=11.2, 5.1 Hz, 10H), 4.03 (dd, J=11.2, 6.3 Hz, 10H), 3.78 (s, 1H), 3.22-2.95 (m, 2H), 2.80-2.40 (m, 3H), 2.36 (t, J=6.9 Hz, 7H), 2.31 (t, J=7.6 Hz, 17H), 2.17 (dqd, J=26.2, 13.5, 12.8, 7.6 Hz, 3H), 1.99 (hept, J=6.0 Hz, 4H), 1.79-1.68 (m, 6H), 1.62 (p, J=7.4 Hz, 26H), 1.38 (d, J=3.7 Hz, 17H), 1.30 (tt, J=13.4, 5.8 Hz, 67H), 0.89 (t, J=6.9 Hz, 24H). ¹³C NMR (151 MHz, CDCl₃) δ 173.83, 173.46, 173.40, 77.26, 77.04, 76.83, 66.29, 66.15, 66.05, 63.85, 53.10, 37.20, 34.27, 33.89, 33.86, 33.82, 33.78, 31.70, 31.69, 31.67, 31.65, 29.12, 29.10, 29.09, 29.07, 28.93, 28.91, 28.89, 28.88, 27.94, 26.35, 26.33, 26.29, 24.96, 24.94, 24.93, 24.92, 24.90, 24.89, 22.60, 22.58, 22.57, 14.11, 14.10, 14.08. MALDI-MS m/z [M+H]⁺ calcd for [C₁₁₈H₂₁₇N₄O₂₈]⁺ 2138.5679, obs 2138.57.

AMG108. ¹H NMR (600 MHz, CDCl₃) δ 4.16-3.95 (m, 14H), 3.95-3.58 (m, 3H), 3.20-2.94 (m, 2H), 2.94-2.47 (m, 4H), 2.45-2.34 (m, 8H), 2.31 (qd, J=9.3, 8.3, 3.7 Hz, 5H), 2.16 (ddtd, J=31.9, 19.3, 13.5, 5.9 Hz, 3H), 1.88-1.61 (m, 20H), 1.58 (dt, J=15.3, 7.1 Hz, 8H), 1.42 (ddd, J=23.5, 12.0, 5.6 Hz, 17H), 1.27 (dh, J=28.2, 7.4 Hz, 50H), 0.87 (td, J=7.1, 2.9 Hz, 24H). ¹³C NMR (151 MHz, CDCl₃) δ 176.63, 173.45, 173.40, 173.37, 69.60, 68.64, 66.45, 66.23, 66.09, 66.07, 66.01, 65.10, 63.76, 63.72, 58.27, 57.62, 53.44, 53.35, 53.25, 53.10, 50.89, 45.87, 45.77, 33.94, 33.91, 33.87, 33.83, 32.51, 32.48, 32.20, 32.17, 31.69, 29.64, 29.62, 29.21, 29.19, 28.43, 28.41, 27.41, 25.53, 25.51, 24.49, 24.47, 24.44, 22.61, 22.58, 22.57, 22.55, 22.54, 14.10, 14.08, 13.99, 13.97, 13.95. HRMS (ESI) m/z [M+H]⁺ calcd for [C₉₄H₁₇₇N₄O₂₀]⁺ 1682.2956, obs 1682.2958.

AMG109. ¹H NMR (600 MHz, CDCl₃) δ 4.16-3.95 (m, 14H), 3.93-3.75 (m, 3H), 3.22-2.97 (m, 2H), 2.85-2.48 (m, 9H), 2.47-2.33 (m, 8H), 2.30 (td, J=9.2, 4.7 Hz, 4H), 2.16 (ddt, J=26.6, 19.8, 9.9 Hz, 4H), 1.83-1.62 (m, 21H), 1.58 (tt, J=12.9, 5.0 Hz, 8H), 1.41 (tt, J=15.4, 7.0 Hz, 18H), 1.27 (d, J=15.9 Hz, 85H), 0.88 (t, J=7.0 Hz, 24H). ¹³C NMR (151 MHz, CDCl₃) δ 176.64, 173.45, 173.38, 66.24, 66.11, 66.02, 63.76, 53.11, 45.90, 45.80, 33.94, 33.87, 33.83, 32.50, 32.47, 31.86, 31.84, 31.69, 29.56, 29.54, 29.44, 29.43, 29.25, 29.24, 29.22, 29.20, 28.43, 27.46, 27.42, 25.54, 25.51, 24.50, 24.48, 24.45, 22.66, 22.59, 22.57, 14.14, 14.12, 14.09. HRMS (ESI) m/z [M+H]⁺ calcd for [C₁₁₀H₂₀₉N₄O₂₀]⁺ 1906.5460, obs 1906.5465.

AMG1010. ¹H NMR (600 MHz, CDCl₃) δ 4.17-3.94 (m, 11H), 2.99-2.48 (m, 5H), 2.37 (t, J=7.5 Hz, 6H), 2.34-2.22 (m, 4H), 1.67 (dt, J=14.9, 7.4 Hz, 16H), 1.58 (tt, J=12.9, 5.3 Hz, 7H), 1.42 (ddd, J=21.1, 10.5, 5.7 Hz, 15H), 1.26 (s, 108H), 0.89 (t, J=7.0 Hz, 24H). ¹³C NMR (151 MHz, CDCl₃) δ 176.67, 173.46, 66.71, 66.05, 63.79, 45.80, 34.31, 34.04, 33.98, 33.95, 33.85, 32.50, 31.98, 31.94, 31.92, 31.90, 31.88, 31.87, 31.85, 29.66, 29.65, 29.61, 29.57, 29.53, 29.52, 29.50, 29.45, 29.44, 29.42, 29.41, 29.39, 29.38, 29.35, 29.33, 29.31, 29.30, 29.28, 29.27, 29.25, 29.23, 29.22, 28.44, 27.58, 27.47, 25.55, 24.63, 24.55, 24.53, 24.51, 24.50, 24.47, 22.69, 22.67, 14.14. MALDI-MS m/z [M+H]⁺ calcd for [C₁₂₆H₂₄₁N₄O₂₀]⁺ 2130.7964, obs 2130.79.

AMG10C12. ¹H NMR (500 MHz, CDCl₃) δ 4.19-3.96 (m, 7H), 3.87 (dddt, J=26.3, 20.6, 10.8, 4.8 Hz, 2H), 3.78-3.60 (m, 1H), 3.25-2.96 (m, 2H), 2.96-2.47 (m, 3H), 2.46-2.26 (m, 10H), 2.24-2.09 (m, 4H), 1.82-1.68 (m, 3H), 1.63 (q, J=7.3 Hz, 11H), 1.29 (d, J=13.9 Hz, 75H), 0.90 (t, J=6.9 Hz, 12H). ¹³C NMR (126 MHz, CDCl₃) δ 173.83, 173.77, 68.67, 66.10, 65.96, 65.90, 65.78, 58.46, 58.31, 57.69, 53.13, 51.70, 50.97, 34.47, 34.43, 34.23, 34.20, 34.18, 34.16, 34.12, 34.07, 31.92, 31.90, 31.88, 29.63, 29.50, 29.48, 29.43, 29.35, 29.31, 29.20, 29.17, 29.13, 25.95, 25.19, 24.95, 24.92, 24.90, 22.69, 22.67, 22.66, 18.45, 14.13, 14.11. HRMS (ESI) m/z [M+H]⁺ calcd for [C₇₀H₁₃₇N₄O₁₂]⁺ 1226.0233, obs 1226.0238.

AMG10C16. ¹H NMR (500 MHz, CDCl₃) δ 4.17-3.96 (m, 6H), 3.95-3.68 (m, 2H), 3.24-2.92 (m, 2H), 2.92-2.48 (m, 3H), 2.48-2.28 (m, 9H), 2.28-2.06 (m, 4H), 1.85-1.52 (m, 14H), 1.27 (s, 97H), 0.90 (t, J=6.8 Hz, 12H). ¹³C NMR (126 MHz, CDCl₃) δ 173.85, 173.83, 173.77, 68.67, 66.11, 65.96, 65.90, 58.31, 57.76, 53.13, 50.97, 34.43, 34.23, 34.18, 34.12, 34.07, 31.94, 29.71, 29.68, 29.65, 29.52, 29.38, 29.35, 29.32, 29.21, 29.19, 29.14, 25.96, 24.95, 24.93, 24.91, 22.71, 14.14. HRMS (ESI) m/z [M+H]⁺ calcd for [C₈₆H₁₆₉N₄O₁₂]⁺ 1450.2737, obs 1450.2741.

AMG124. ¹H NMR (500 MHz, CDCl₃) δ 5.62-5.12 (m, 8H), 4.12 (dh, J=11.1, 5.9 Hz, 2H), 4.04 (dt, J=10.7, 5.5 Hz, 2H), 4.00-3.89 (m, 3H), 3.14-2.65 (m, 3H), 2.65-2.42 (m, 2H), 2.36 (p, J=10.4, 9.3 Hz, 5H), 2.20 (td, J=22.7, 22.0, 10.3 Hz, 2H), 2.03 (q, J=6.5 Hz, 10H), 1.63 (q, J=7.4 Hz, 5H), 1.32 (t, J=14.4 Hz, 50H), 0.90 (t, J=6.8 Hz, 12H). ¹³C NMR (126 MHz, CDCl₃) δ 173.76, 130.01, 129.99, 129.76, 129.74, 68.63, 66.75, 66.58, 66.14, 66.06, 65.89, 65.48, 58.71, 58.27, 54.24, 53.28, 52.50, 51.14, 44.68, 34.18, 34.14, 34.10, 31.92, 29.79, 29.75, 29.55, 29.37, 29.34, 29.27, 29.25, 29.18, 29.15, 29.11, 27.24, 27.21, 24.96, 24.91, 24.88, 22.70, 14.16, 14.14. HRMS (ESI) m/z [M+H]⁺ calcd for [C₉₂H₁₇₃N₄O₁₂]⁺ 1526.3050, obs 1526.3054.

AMG134. ¹H NMR (500 MHz, CDCl₃) δ 5.44-5.25 (m, 6H), 4.22-4.08 (m, 2H), 4.02 (dddd, J=13.6, 9.8, 6.6, 3.1 Hz, 3H), 3.83-3.71 (m, 1H), 2.87-2.45 (m, 4H), 2.42-2.19 (m, 7H), 2.03 (q, J=6.5 Hz, 15H), 1.66 (dq, J=14.9, 7.3 Hz, 10H), 1.44-1.11 (m, 69H), 0.90 (t, J=6.8 Hz, 9H). ¹³C NMR (126 MHz, CDCl₃) δ 173.82, 130.06, 130.03, 130.01, 129.76, 129.74, 129.72, 71.35, 66.65, 66.56, 66.37, 65.64, 65.40, 64.34, 58.58, 56.04, 49.28, 34.44, 34.20, 34.18, 31.94, 31.92, 29.78, 29.72, 29.54, 29.52, 29.46, 29.33, 29.29, 29.21, 29.16, 29.14, 27.24, 27.19, 27.17, 24.95, 24.92, 23.62, 23.51, 22.70, 22.68, 14.13, 14.11. HRMS (ESI) m/z [M+H]⁺ calcd for [C₇₂H₁₃₆N₃₀₉]⁺ 1187.0277, obs 1187.0279.

AMG1041. ¹H NMR (500 MHz, CDCl₃) δ 5.41 (dt, J=14.3, 7.4 Hz, 4H), 5.33 (dd, J=13.0, 5.9 Hz, 5H), 4.18-3.96 (m, 7H), 3.96-3.58 (m, 3H), 3.06 (tdd, J=32.3, 16.8, 9.2 Hz, 2H), 2.90-2.46 (m, 3H), 2.36 (qt, J=12.9, 5.6 Hz, 9H), 2.26-2.12 (m, 3H), 2.08 (q, J=7.4 Hz, 8H), 2.01 (q, J=7.0 Hz, 9H), 1.81-1.57 (m, 13H), 1.44-1.12 (m, 35H), 0.89 (t, J=6.6 Hz, 12H). ¹³C NMR (126 MHz, CDCl₃) δ 173.65, 173.57, 131.25, 131.20, 131.18, 131.16, 131.13, 131.10, 128.37, 128.33, 128.31, 128.29, 128.22, 68.64, 66.34, 66.16, 66.02, 65.96, 65.91, 65.87, 58.31, 57.69, 53.38, 53.28, 53.11, 51.69, 50.95, 33.86, 33.82, 33.63, 33.60, 33.56, 33.54, 33.52, 33.50, 33.48, 31.78, 29.67, 28.99, 27.25, 26.56, 26.51, 25.95, 25.18, 24.90, 24.87, 22.68, 22.65, 14.17, 14.15, 14.12, 14.10. HRMS (ESI) m/z [M+H]⁺ calcd for [C₇₀H₁₂₉N₄O₁₂]⁺ 1217.9607, obs 1217.9609.

AMG1042. ¹H NMR (500 MHz, CDCl₃) δ 5.44-5.26 (m, 8H), 4.18-3.95 (m, 7H), 3.94 (s, 0H), 3.23-2.94 (m, 2H), 2.94-2.47 (m, 3H), 2.43-2.28 (m, 9H), 2.17 (dqd, J=25.8, 11.0, 10.1, 5.6 Hz, 3H), 2.03 (dq, J=9.6, 6.1, 4.7 Hz, 17H), 1.83-1.52 (m, 14H), 1.43-1.17 (m, 51H), 0.99-0.82 (m, 12H). ¹³C NMR (126 MHz, CDCl₃) δ 173.81, 173.79, 173.74, 173.72, 129.96, 129.74, 129.73, 68.66, 66.29, 66.11, 65.97, 65.92, 65.86, 65.81, 58.30, 57.68, 53.38, 53.28, 53.12, 51.70, 50.96, 34.40, 34.20, 34.17, 34.15, 34.13, 34.09, 34.05, 34.02, 31.96, 31.92, 29.71, 29.22, 29.19, 29.15, 29.12, 27.16, 26.97, 26.96, 26.92, 25.95, 25.19, 24.92, 24.90, 24.88, 22.35, 22.31, 14.02, 13.98. HRMS (ESI) m/z [M+H]⁺ calcd for [C₇₈H₁₄₅N₄O₁₂]⁺ 1330.0859, obs 1330.0861.

AMG1043. ¹H NMR (500 MHz, CDCl₃) δ 5.45-5.26 (m, 8H), 4.06 (dddd, J=24.0, 21.4, 11.6, 6.4 Hz, 6H), 3.96-3.63 (m, 2H), 3.21-2.91 (m, 2H), 2.87-2.42 (m, 3H), 2.42 (s, 0H), 2.17 (dtdd, J=26.0, 15.4, 11.0, 4.6 Hz, 4H), 2.02 (q, J=6.5 Hz, 18H), 1.82-1.54 (m, 13H), 1.31 (dt, J=11.3, 5.9 Hz, 72H), 0.90 (t, J=6.8 Hz, 12H). ¹³C NMR (126 MHz, CDCl₃) δ 173.81, 173.79, 173.74, 173.72, 130.02, 129.74, 129.72, 68.66, 66.29, 66.12, 65.98, 65.92, 65.81, 58.30, 57.76, 53.28, 53.12, 51.69, 50.96, 34.20, 34.17, 34.15, 34.13, 34.09, 34.05, 31.79, 29.74, 29.72, 29.24, 29.21, 29.16, 29.14, 29.10, 28.99, 27.23, 27.18, 24.92, 24.90, 24.88, 22.67, 14.12. HRMS (ESI) m/z [M+H]⁺ calcd for [C₈₆H₁₆₁N₄O₁₂]⁺ 1442.2111, obs 1442.2111.

AMG1044. ¹H NMR (500 MHz, CDCl₃) δ 5.45-5.24 (m, 8H), 4.19-3.94 (m, 7H), 3.90 (d, J=33.3 Hz, 1H), 3.07 (tdd, J=34.3, 17.6, 7.3 Hz, 2H), 2.89-2.49 (m, 3H), 2.49-2.27 (m, 9H), 2.25-2.09 (m, 3H), 2.03 (q, J=6.5 Hz, 17H), 1.85-1.54 (m, 13H), 1.50-1.09 (m, 86H), 0.90 (t, J=6.7 Hz, 12H). ¹³C NMR (126 MHz, CDCl₃) δ 173.81, 173.74, 129.94, 129.83, 68.66, 66.28, 66.11, 65.97, 65.91, 65.80, 58.30, 57.69, 53.38, 53.29, 53.12, 51.70, 50.96, 34.42, 34.21, 34.18, 34.17, 34.15, 34.11, 34.06, 34.03, 31.84, 31.83, 31.79, 29.78, 29.75, 29.67, 29.64, 29.63, 29.52, 29.48, 29.33, 29.30, 29.20, 29.18, 29.13, 29.10, 29.09, 29.07, 29.06, 29.04, 29.03, 28.99, 28.98, 28.95, 28.93, 28.92, 27.38, 27.35, 27.34, 27.32, 27.31, 27.23, 27.21, 27.12, 25.95, 25.19, 24.94, 24.91, 24.89, 22.72, 22.70, 22.69, 22.67, 22.63, 22.62, 14.12. HRMS (ESI) m/z [M+H]⁺ calcd for [C₉₄H₁₇₇N₄O₁₂]⁺ 1554.3363, obs 1554.3366.

AMG1047. ¹H NMR (600 MHz, CDCl₃) δ 5.34 (tdt, J=18.6, 12.0, 6.8 Hz, 8H), 4.13-3.94 (m, 7H), 3.93-3.61 (m, 5H), 3.15-2.91 (m, 1H), 2.88-2.45 (m, 2H), 2.43-2.24 (m, 9H), 2.13 (dq, J=14.2, 10.1, 8.3 Hz, 3H), 1.99 (ddq, J=27.7, 20.0, 7.0 Hz, 17H), 1.81-1.54 (m, 10H), 1.43-1.12 (m, 32H), 0.87 (td, J=7.0, 3.1 Hz, 12H). ¹³C NMR (151 MHz, CDCl₃) δ 173.68, 173.66, 173.60, 130.49, 129.05, 68.64, 66.12, 65.98, 65.93, 65.83, 63.35, 58.30, 57.75, 53.38, 53.28, 53.12, 51.69, 50.95, 34.35, 34.30, 34.16, 34.10, 34.07, 34.06, 34.04, 34.02, 34.00, 33.96, 32.56, 32.21, 32.19, 31.55, 31.52, 31.50, 31.47, 31.41, 31.30, 29.70, 29.67, 29.40, 29.38, 29.36, 29.35, 29.33, 29.32, 29.28, 29.24, 29.22, 29.18, 29.10, 29.08, 27.20, 27.17, 26.85, 25.94, 25.18, 24.56, 24.54, 24.41, 22.62, 22.57, 22.54, 22.33, 14.13, 14.12, 14.09, 14.07, 14.04, 14.02, 13.93. ESI-MS m/z [M+H]⁺ calcd for [C₇₀H₁₂₉N₄O₁₂]⁺ 1217.9607, obs 1217.9622.

AMG1048. ¹H NMR (600 MHz, CDCl₃) δ 5.41-5.28 (m, 8H), 4.14-3.96 (m, 7H), 3.96-3.58 (m, 3H), 3.17-2.95 (m, 1H), 2.57 (ddt, J=40.8, 19.4, 6.4 Hz, 1H), 2.40-2.28 (m, 8H), 2.23-2.09 (m, 3H), 2.02 (h, J=5.8, 5.4 Hz, 14H), 1.98 (q, J=6.6 Hz, 2H), 1.76 (s, 1H), 1.72-1.68 (m, 2H), 1.68-1.60 (m, 9H), 1.34 (ddt, J=17.8, 7.1, 3.9 Hz, 34H), 0.91 (dtd, J=7.2, 5.1, 1.9 Hz, 12H). ¹³C NMR (151 MHz, CDCl₃) δ 173.73, 173.68, 130.16, 130.14, 129.45, 129.43, 70.23, 68.64, 66.34, 66.29, 66.11, 65.97, 65.92, 65.81, 65.18, 63.35, 58.30, 57.71, 53.37, 53.28, 53.11, 51.69, 50.95, 34.36, 34.16, 34.13, 34.11, 34.09, 34.05, 34.00, 33.98, 32.38, 32.26, 31.93, 31.89, 31.88, 31.80, 29.69, 29.42, 29.40, 29.36, 29.26, 28.81, 28.79, 28.75, 28.73, 28.66, 28.63, 27.00, 26.98, 26.96, 26.94, 26.92, 26.89, 26.88, 25.93, 25.18, 24.83, 24.80, 24.78, 22.33, 22.30, 22.29, 22.26, 22.24, 22.21, 22.19, 14.01, 13.99, 13.96, 13.73. ESI-MS m/z [M+H]⁺ calcd for [C₇₀H129N₄O₁₂]⁺ 1217.9607, obs 1217.9611.

AMG1049. ¹H NMR (600 MHz, CDCl₃) δ 5.41-5.32 (m, 8H), 4.15-3.96 (m, 6H), 3.92-3.75 (m, 1H), 3.12 (d, J=8.8 Hz, 1H), 3.06 (t, J=8.9 Hz, 1H), 2.81-2.46 (m, 1H), 2.35 (dtd, J=9.4, 7.6, 4.7 Hz, 7H), 2.22-2.10 (m, 1H), 2.01 (ddd, J=14.8, 7.3, 5.3 Hz, 14H), 1.78-1.67 (m, 2H), 1.63 (qd, J=9.6, 9.2, 4.6 Hz, 10H), 1.42-1.21 (m, 34H), 1.25 (s, 7H), 0.94-0.86 (m, 12H). ¹³C NMR (151 MHz, CDCl₃) δ 173.78, 173.77, 173.71, 129.84, 129.82, 129.80, 71.12, 68.65, 66.28, 66.11, 65.97, 65.91, 65.85, 65.81, 63.35, 58.30, 57.75, 57.67, 53.38, 53.28, 53.12, 51.69, 50.95, 34.70, 34.44, 34.39, 34.18, 34.16, 34.14, 34.12, 34.08, 34.03, 34.01, 32.51, 29.69, 29.57, 29.44, 29.41, 29.36, 29.34, 29.33, 29.29, 29.26, 29.14, 29.08, 29.05, 29.03, 29.01, 28.94, 28.92, 28.91, 28.89, 28.79, 28.76, 27.14, 25.94, 25.18, 24.89, 24.87, 24.85, 22.91, 22.88, 22.84, 22.73, 13.87, 13.82, 13.66. ESI-MS m/z [M+H]⁺ calcd for [C₇₀H₁₂₉N₄O₁₂]⁺ 1217.9607, obs 1217.9612.

AMG1050. ¹H NMR (600 MHz, CDCl₃) δ 5.34 (tdd, J=17.9, 11.0, 6.9 Hz, 8H), 4.14-3.97 (m, 6H), 3.90-3.78 (m, 2H), 3.12 (s, 1H), 3.06 (dd, J=16.1, 7.1 Hz, 1H), 2.80-2.50 (m, 12H), 2.41 (ddt, J=16.0, 10.1, 2.7 Hz, 1H), 2.34 (h, J=6.8, 6.2 Hz, 8H), 2.22-2.09 (m, 3H), 2.03 (dq, J=14.4, 7.3 Hz, 15H), 1.75 (s, 1H), 1.70 (q, J=6.5 Hz, 1H), 1.62 (q, J=7.3 Hz, 10H), 1.39-1.23 (m, 8H), 0.96 (t, J=7.5 Hz, 12H). ¹³C NMR (151 MHz, CDCl₃) δ 173.80, 173.78, 173.72, 131.61, 129.18, 129.17, 68.65, 66.27, 66.10, 65.97, 65.91, 65.85, 65.80, 63.35, 58.29, 57.67, 53.37, 53.27, 53.11, 51.69, 50.96, 34.39, 34.19, 34.17, 34.15, 34.13, 34.09, 34.04, 34.01, 32.52, 29.69, 29.65, 29.58, 29.20, 29.17, 29.14, 29.12, 29.08, 28.98, 27.05, 25.94, 25.59, 25.18, 24.91, 24.89, 24.87, 20.54, 20.51, 14.40, 14.37, 14.01. ESI-MS m/z [M+H]⁺ calcd for [C₇₀H₁₂₉N₄O₁₂]⁺ 1217.9607, obs 1217.9620.

AMG1051. ¹H NMR (600 MHz, CDCl₃) δ 5.41 (dddd, J=25.5, 18.2, 11.6, 6.7 Hz, 8H), 4.05 (dddd, J=30.8, 16.0, 12.9, 8.2 Hz, 6H), 3.95-3.55 (m, 3H), 3.16-3.02 (m, 2H), 2.87-2.48 (m, 2H), 2.44-2.27 (m, 9H), 2.16 (ddt, J=20.2, 13.4, 8.1 Hz, 3H), 2.03 (q, J=7.1 Hz, 8H), 1.79-1.67 (m, 3H), 1.66-1.62 (m, 8H), 1.61 (d, J=6.7 Hz, 13H), 1.41-1.09 (m, 46H). ¹³C NMR (151 MHz, CDCl₃) δ 173.80, 173.74, 130.79, 123.65, 70.23, 68.65, 66.26, 66.09, 65.96, 65.90, 65.84, 65.79, 63.35, 62.98, 58.29, 57.67, 53.37, 53.28, 53.12, 51.69, 50.95, 34.45, 34.40, 34.20, 34.18, 34.16, 34.14, 34.10, 34.05, 34.03, 32.58, 29.69, 29.58, 29.53, 29.45, 29.37, 29.35, 29.31, 29.28, 29.26, 29.22, 29.17, 29.14, 29.12, 29.09, 26.81, 25.93, 25.18, 24.92, 24.90, 24.88, 17.93, 12.76. ESI-MS m/z [M+H]⁺ calcd for [C₇₀H₁₂₉N₄O₁₂]⁺ 1217.9607, obs 1217.9611.

AMG1053. ¹H NMR (500 MHz, CDCl₃) δ 5.48-5.28 (m, 16H), 4.18-3.97 (m, 6H), 3.96-3.59 (m, 5H), 3.17-2.96 (m, 2H), 2.81 (t, J=6.9 Hz, 7H), 2.76-2.50 (m, 3H), 2.45-2.36 (m, 16H), 2.33 (d, J=6.3 Hz, 2H), 2.24-1.98 (m, 12H), 1.88-1.52 (m, 4H), 1.34 (dh, J=7.3, 3.8 Hz, 16H), 0.91 (td, J=7.2, 2.9 Hz, 13H). ¹³C NMR (126 MHz, CDCl₃) δ 173.14, 173.11, 173.08, 173.06, 130.55, 130.52, 129.77, 129.74, 127.57, 127.55, 127.48, 127.45, 127.43, 66.42, 66.24, 66.05, 65.91, 63.31, 57.72, 53.11, 34.30, 34.10, 34.03, 33.98, 33.96, 33.94, 31.81, 31.68, 30.35, 27.79, 26.95, 26.84, 25.60, 22.75, 22.72, 22.34, 22.32, 22.21, 14.07, 14.02, 13.99. ESI-MS m/z [M+H]⁺ calcd for [C₇₀H₁₂₁N₄O₁₂]⁺ 1209.8981, obs 1209.8990.

AMG1054. ¹H NMR (500 MHz, CDCl₃) δ 5.52-5.19 (m, 16H), 4.15-3.95 (m, 4H), 3.83 (s, 3H), 3.80 (d, J=3.5 Hz, OH), 3.09 (dt, J=34.6, 9.4 Hz, 2H), 2.77 (t, J=6.7 Hz, 6H), 2.43-2.29 (m, 6H), 2.12 (tdd, J=12.9, 8.2, 4.4 Hz, 8H), 2.04 (q, J=7.0 Hz, 7H), 1.71 (p, J=7.5 Hz, 7H), 1.44-1.29 (m, 6H), 0.91 (td, J=7.4, 4.5 Hz, 9H). ¹³C NMR (126 MHz, CDCl₃) δ 173.59, 173.51, 130.16, 129.25, 129.22, 128.66, 128.63, 128.61, 127.85, 127.83, 66.36, 66.18, 66.04, 65.98, 63.35, 58.28, 57.72, 53.38, 53.11, 51.66, 50.94, 33.78, 33.59, 33.56, 33.53, 33.51, 33.48, 33.47, 33.45, 31.88, 30.40, 29.29, 29.26, 29.25, 29.20, 26.55, 26.51, 25.63, 25.61, 25.15, 24.79, 24.77, 24.76, 24.64, 22.77, 22.63, 13.82, 13.78, 13.76. ESI-MS m/z [M+H]⁺ calcd for [C₇₀H₁₂₁N₄O₁₂]⁺ 1209.8981, obs 1209.9112.

AMG1055. ¹H NMR (500 MHz, CDCl₃) δ 5.45-5.25 (m, 16H), 4.15-3.95 (m, 8H), 3.86 (s, 3H), 3.09 (dt, J=34.8, 9.3 Hz, 3H), 2.78 (t, J=6.1 Hz, 8H), 2.75-2.54 (m, 3H), 2.36 (qd, J=7.5, 6.3, 3.7 Hz, 10H), 2.23-1.96 (m, 18H), 1.76-1.57 (m, 12H), 1.48-1.34 (m, 10H), 1.28 (d, J=11.0 Hz, 1H), 1.03-0.87 (m, 13H). 13C NMR (126 MHz, CDCl₃) δ 173.66, 173.64, 173.58, 131.90, 129.39, 129.37, 128.56, 128.53, 128.52, 127.22, 127.21, 74.09, 70.23, 68.64, 66.32, 66.14, 66.00, 65.95, 65.86, 63.35, 58.30, 57.68, 53.38, 53.28, 53.12, 51.68, 50.93, 34.97, 34.28, 34.07, 34.03, 33.98, 33.94, 32.15, 30.31, 29.13, 29.11, 29.07, 28.97, 26.86, 26.84, 25.54, 25.50, 24.55, 24.53, 24.41, 22.55, 20.55, 20.45, 14.30, 14.29, 13.76. ESI-MS m/z [M+H]⁺ calcd for [C₇₀H₁₂₁N₄O₁₂]⁺ 1209.8981, obs 1209.9113.

AMG1056. ¹H NMR (500 MHz, CDCl₃) δ 5.51-5.42 (m, 4H), 5.42-5.28 (m, 12H), 4.12-3.98 (m, 6H), 3.96-3.77 (m, 2H), 3.84 (s, 4H), 3.08 (tt, J=20.6, 7.4 Hz, 3H), 2.78 (t, J=6.5 Hz, 7H), 2.76-2.49 (m, 3H), 2.34 (pd, J=8.2, 7.4, 2.6 Hz, 10H), 2.24-2.11 (m, 3H), 2.07 (q, J=6.6 Hz, 8H), 1.72 (s, 3H), 1.75-1.60 (m, 20H), 1.43-1.29 (m, 18H). ¹³C NMR (126 MHz, CDCl₃) δ 173.74, 173.72, 173.66, 129.83, 129.81, 129.79, 128.85, 128.10, 128.08, 124.01, 68.65, 66.31, 66.13, 65.99, 65.93, 65.83, 63.35, 58.29, 57.73, 53.37, 53.27, 53.12, 51.67, 50.94, 34.40, 34.35, 34.14, 34.12, 34.10, 34.08, 34.04, 33.99, 32.34, 30.44, 30.07, 29.32, 29.29, 29.26, 29.14, 28.80, 28.78, 28.73, 27.03, 26.91, 25.93, 25.89, 25.33, 25.29, 25.28, 25.17, 24.81, 24.79, 24.77, 17.93, 12.79, 12.77. ESI-MS m/z [M+H]⁺ calcd for [C₇₀H₁₂₁N₄O₁₂]⁺ 1209.8981, obs 1209.8995.

AMG1058. ¹H NMR (600 MHz, CDCl₃) δ 5.41 (dtd, J=10.2, 7.6, 7.1, 1.3 Hz, 4H), 5.37-5.30 (m, 3H), 4.48-4.04 (m, 8H), 2.79 (s, 2H), 2.11-2.06 (m, 7H), 2.03 (q, J=7.1 Hz, 11H), 1.78-1.74 (m, 5H), 1.68 (ddd, J=12.4, 5.0, 3.4 Hz, 5H), 1.47-1.39 (m, 11H), 1.38-1.23 (m, 29H), 0.91 (td, J=7.0, 2.3 Hz, 12H). ¹³C NMR (126 MHz, CDCl₃) δ 173.74, 173.72, 130.71, 130.69, 128.92, 128.89, 70.29, 65.18, 63.35, 57.38, 34.10, 31.79, 29.69, 29.00, 28.19, 28.17, 27.28, 26.71, 25.95, 25.92, 22.66, 24.77, 17.93, 14.12, 12.77. ESI-MS m/z [M+H]⁺ calcd for [C₇₀H₁₂₉N₄O₁₂]⁺ 1217.9607, obs 1217.9610.

AMG1059. ¹H NMR (600 MHz, CDCl₃) δ 5.44-5.37 (m, 4H), 5.37-5.30 (m, 4H), 4.08 (t, J=6.8 Hz, 8H), 2.76 (t, J=7.4 Hz, 9H), 2.53-2.47 (m, 16H), 2.43 (t, J=7.3 Hz, 8H), 2.05 (dqd, J=28.0, 7.3, 1.4 Hz, 18H), 1.71-1.61 (m, 9H), 1.58 (q, J=6.9 Hz, 4H), 1.47-1.39 (m, 9H), 1.39-1.33 (m, 4H), 1.35-1.24 (m, 17H), 0.93-0.87 (m, 13H). ¹³C NMR (151 MHz, CDCl₃) δ 172.86, 130.60, 129.02, 64.40, 51.25, 51.17, 50.60, 32.60, 32.58, 31.78, 31.77, 31.75, 29.70, 28.99, 28.98, 28.26, 28.24, 27.27, 27.25, 26.76, 26.74, 26.05, 26.03, 24.00, 22.66, 22.64, 14.12, 14.10. ESI-MS m/z [M+H]⁺ calcd for [C₇₀H₁₂₉N_{408]}⁺ 1153.9810, obs 1153.9820.

AMG1041-OHD. 1H NMR (600 MHz, CDCl₃) δ 5.46-5.38 (m, 4H), 5.38-5.30 (m, 4H), 4.12 (t, J=6.6 Hz, 8H), 2.51 (d, J=7.7 Hz, 15H), 2.46 (t, J=7.2 Hz, 8H), 2.32 (t, J=7.6 Hz, 8H), 2.09 (qd, J=7.3, 1.5 Hz, 8H), 2.02 (qd, J=7.2, 1.5 Hz, 8H), 1.73 (dq, J=35.3, 7.1 Hz, 15H), 1.60 (p, J=7.0 Hz, 4H), 1.39-1.30 (m, 8H), 1.29 (dtd, J=9.1, 6.4, 3.5 Hz, 12H), 0.90 (t, J=7.0 Hz, 10H). ¹³C NMR (151 MHz, CDCl₃) δ 173.68, 131.14, 131.12, 128.36, 128.35, 62.80, 51.79, 51.54, 51.22, 33.78, 31.78, 29.67, 28.99, 28.97, 27.25, 26.74, 26.59, 24.98, 23.62, 22.66, 14.14, 14.12. ESI-MS m/z [M+H]⁺ calcd for [C₇₀H₁₂₉N₄₀₈]⁺1153.9810, obs 1153.9865.

### Synthesis of LNPs

LNPs for in vitro studies and intramuscular injections were prepared by hand mixing. The lipid components were dissolved in ethanol and, separately, mRNA encoding for firefly luciferase (fLuc) was diluted into a solution of 10 mM citrate pH 3.0 at a concentration of 133 ng/L. The two solutions were mixed in a 3:1 water:ethanol volume ratio by repeatedly pipetting up and down. For intravenous studies, nanoparticles were instead formed by mixing the ethanol and aqueous phases with a microfluidic chip as described previously.

### Evaluation of the LNPs In Vitro

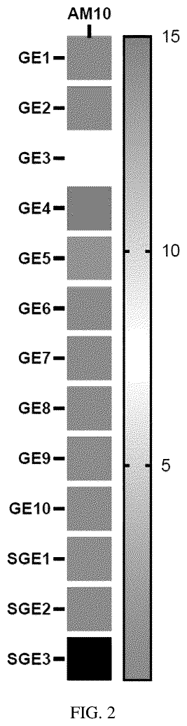

HeLa cells were plated on a white, flat-bottom 96 well plate (Corning, Corning, NY) at a density of 15k cells/well and allowed to recover for 24 hrs. Each well was then dosed with 150 ng of mRNA, and, after 24 hrs., luciferase expression was determined using a Bright-Glo assay (Promega, Madison, WI). The first generation of ionizable lipids was screened in this manner and the results are shown as performance relative to a positive control MD-1³ in a heatmap in FIG. 2.

### Evaluation of the LNPs for T Cell Transfection

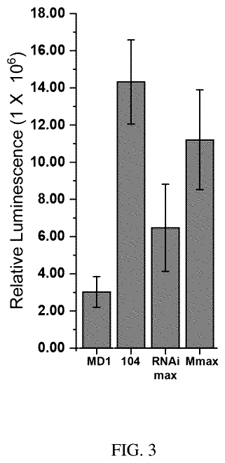

Based on high transfection potency in Hela cells, some of the potent lipids were tested for mRNA delivery efficacy in Jurkat cells, an immortalized human T-cell line for potential CAR-T therapeutic applications. Jurkat cells were cultured in a RPMI-1640 medium with L-glutamine supplemented with 10% bovine serum and 1% penicillin-streptomycin. Jurkat cells were plated on a white, flat-bottom 96 well plate (Corning, Corning, NY) at a density of 100k cells/well and co-incubated dosed with 150 ng of mRNA. Luciferase expression in the cells was determined after 24 hours using a Bright-Glo assay (Promega, Madison, WI) and normalized to cell viability (Multi-tox, Promega). The ionizable lipid AMG104 demonstrated higher mRNA transfection to commercially used lipofectamine positive controls (FIG. 3). Importantly, AMG104 showed increased transfection in jurkat cells as compared to MD-1 lipid, which is one of the most potent lipids used for mRNA delivery.

### Evaluation of the LNPs Via Intramuscular Injection

The in vivo potency of the ionizable lipids was screened through intramuscular injection of LNPs loaded with mRNA encoding firefly luciferase protein. These LNPs were formulated with a molar composition of (35:16:46.5:2.5) using one of the synthesized ionizable lipids, DOPE as a helper lipid, cholesterol, and DMPE-PEG. Ionizable lipid AMG104 was chosen due to high in vitro mRNA transfection ability. mRNA-loaded LNPs were intramuscularly administered in mice at a dose of 0.25 mg/kg (5 μg) and expression of luciferase protein was monitored at 6 hours (0.25 day), and day 1, 2, 3, 5 and 7. LNPs formulated with highly potent, non-degradable MD-1 ionizable lipid with same molar composition was used as a positive control. Lipid AMG104 elicited high luciferase protein expression in mice as compared to the other lipids and their potency was comparable to that of MD1, which is one of the most potent ionizable lipid for mRNA delivery. Other ionizable lipids also showed potent luciferase expression, showing high correlation between the in vitro and intramuscular potency of the ionizable lipids.

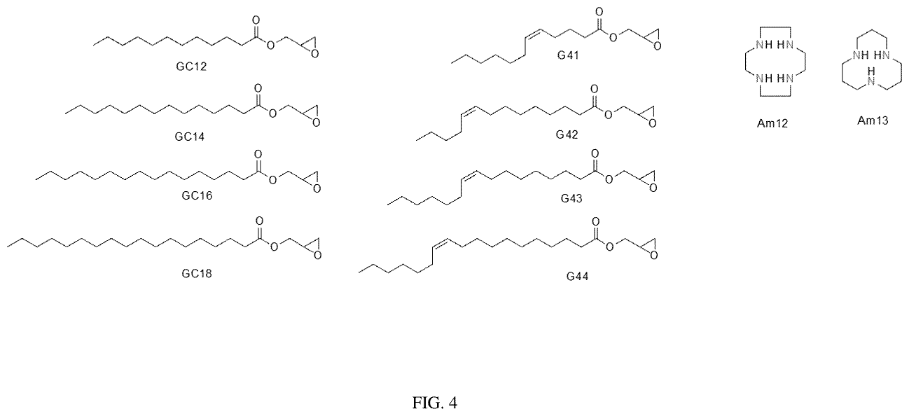

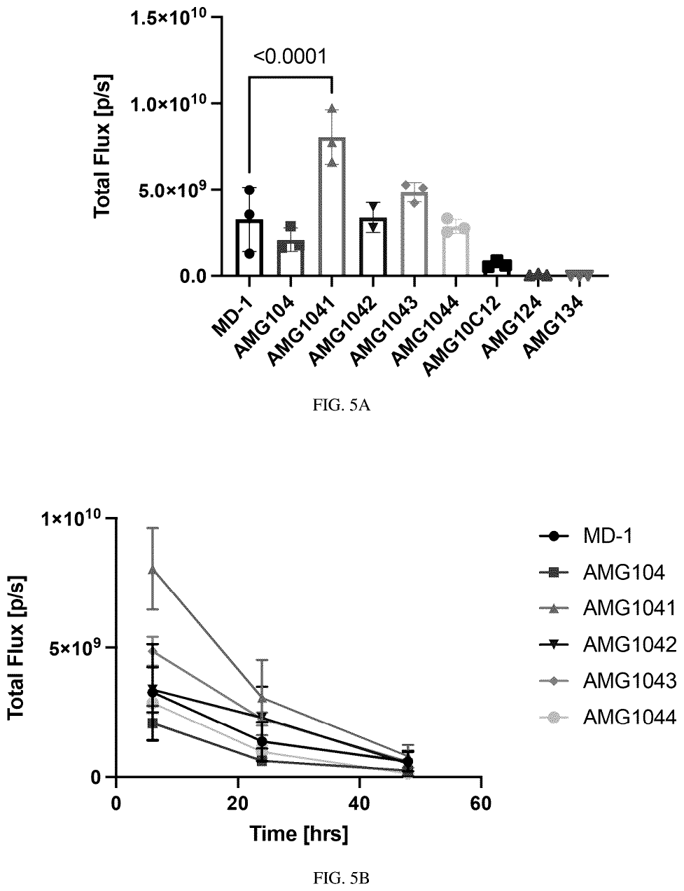

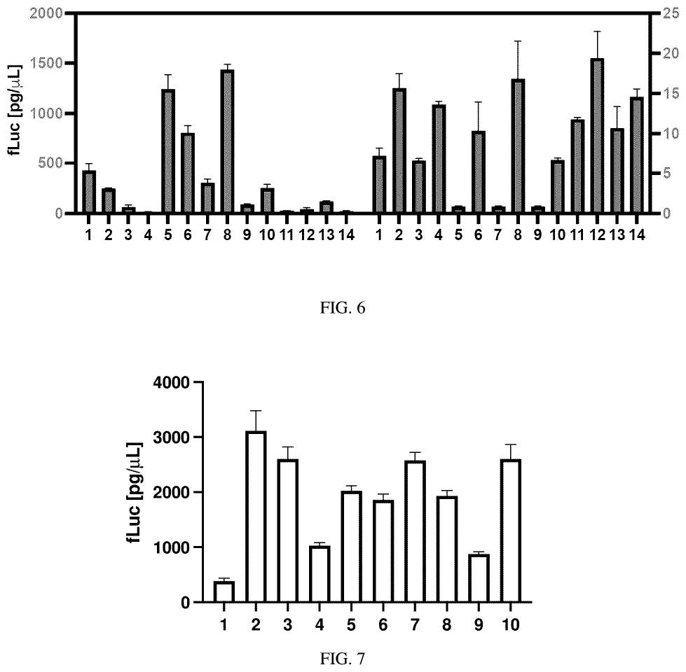

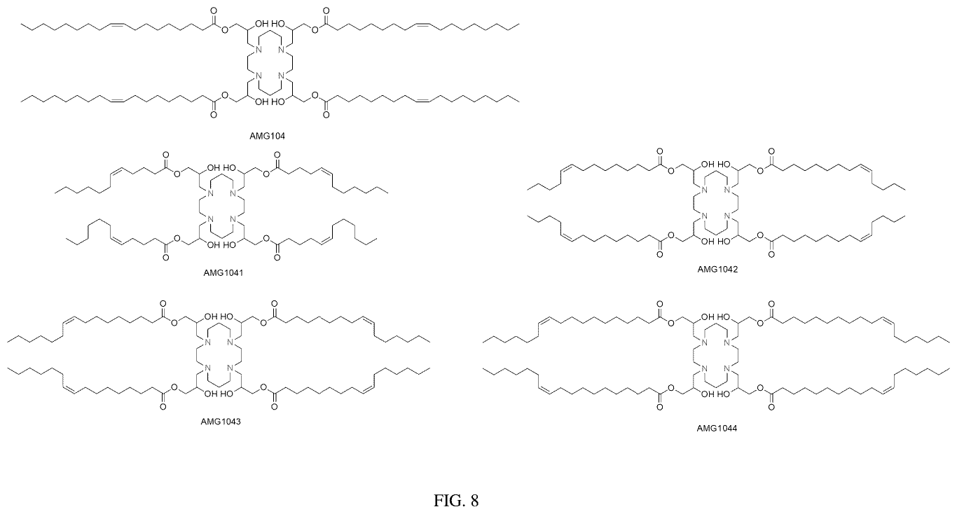

Additional lipids were synthesized varying length of the hydrophobic tail with or without one alkene or by varying the location of the internal alkene near or away from the ester linkage. This resulted in another 8 lipids with the tails shown in FIG. 4. The number of carbons on the amine headgroup was also varied (FIG. 4). These headgroups in the lipids AMG124 and AM134 produced lower luciferase expression and luminescence (quantified as total flux) (FIG. 5A). As shown in FIG. 5A, lipid AMG1041 stood out and exhibited 2-3 times higher luciferase expression while lipids AMG1042, 1043, and 1044 were nearly as potent as one of the validated top performing lipids, MD-1. The luciferase expression was monitored at 6 h, 24 h, and 48 h time points after intramuscular administration of Fluc mRNA dosed at 0.25 mg/kg formulated with various LNPs containing these lipids.

### Design of Experiment (DOE) Optimization of the Formulations for the LNPs

For intravenous experiments, design of experiments (DOE) was used to systematically vary the ratios of each of the LNP components for lipid AMG104. First, an initial screening of each of the components was performed using a definitive screening design. This experimental design allows for identification of the LNP components which have the greatest effect on delivery potency. Two standard control formulations were included for reference, shown as DOPE formulation 1 and DSPC formulation 1 in Tables 1 and 2 below. Separate screens were performed for each of the helper lipids DOPE and DSPC. The composition of each formulation is shown in Table 1 below. All screening was performed in vitro as described above.

TABLE 1
Range of parameters used to create the
definitive screening for lipid AMG104

| | DOPE/DSPC |
|---|---|
| Parameter | Screening Value |
| Ionizable lipid:mRNA weight ratio | 5:1 to 15:1 |
| Ionizable lipid molar % | 30% to 60% |
| Phospholipid molar % | 10% to 30% |
| DMPE-PEG2k molar % | 1.5% to 3% |
| Cholesterol molar % | Rest |

TABLE 2
Definitive screening formulations for lipid AMG104

| | | | | | |
|---|---|---|---|---|---|
| | | | Ionizable | | ionizable |
| Formu- | Helper | DMPE-PEG | Lipid | Cholesterol | lipid:mRNA |
| lation | % | % | % | % | ratio |

DOPE Helper Lipid

| | | | | | |
|---|---|---|---|---|---|
| 1 | 16 | 2.5 | 35 | 46.5 | 10 |
| 2 | 20 | 2.25 | 45 | 32.75 | 10 |
| 3 | 20 | 3 | 60 | 17 | 15 |
| 4 | 10 | 2.25 | 60 | 27.75 | 5 |
| 5 | 30 | 3 | 30 | 37 | 10 |
| 6 | 20 | 1.5 | 30 | 48.5 | 5 |
| 7 | 30 | 1.5 | 45 | 23.5 | 5 |
| 8 | 30 | 2.25 | 30 | 37.75 | 15 |
| 9 | 30 | 3 | 60 | 7 | 5 |
| 10 | 30 | 1.5 | 60 | 8.5 | 15 |
| 11 | 10 | 1.5 | 60 | 28.5 | 10 |
| 12 | 10 | 3 | 30 | 57 | 5 |
| 13 | 10 | 1.5 | 30 | 58.5 | 15 |
| 14 | 10 | 3 | 45 | 42 | 15 |

DSPC Helper Lipid

| | | | | | |
|---|---|---|---|---|---|
| 1 | 10 | 1.5 | 50 | 38.5 | 5 |
| 2 | 20 | 2.25 | 45 | 32.75 | 10 |
| 3 | 20 | 3 | 60 | 17 | 15 |
| 4 | 10 | 2.25 | 60 | 27.75 | 5 |
| 5 | 30 | 3 | 30 | 37 | 10 |
| 6 | 20 | 1.5 | 30 | 48.5 | 5 |
| 7 | 30 | 1.5 | 45 | 23.5 | 5 |
| 8 | 30 | 2.25 | 30 | 37.75 | 15 |
| 9 | 30 | 3 | 60 | 7 | 5 |
| 10 | 30 | 1.5 | 60 | 8.5 | 15 |
| 11 | 10 | 1.5 | 60 | 28.5 | 10 |
| 12 | 10 | 3 | 30 | 57 | 5 |
| 13 | 10 | 1.5 | 30 | 58.5 | 15 |
| 14 | 10 | 3 | 45 | 42 | 15 |

For lipid AMG104, additional in vitro optimization was performed using DOE. Analysis of the definitive screening results revealed that of the effects screen, increasing helper lipid content and decreasing ionizable lipid content had the largest effect on delivery potency. A full factorial design was used to vary ionizable lipid content and helper lipid content. The design was shifted in the direction of the optimum identified in the definitive screening experiment. Other components were set to reasonable values—the DMPE-PEG content was set to 2.5% and the ionizable lipid:mRNA weight ratio was set to 10:1. The experimental design is shown in Tables 3 and 4 below.

TABLE 3
Range of parameters used to create the
full factorial DOE for lipid AMG104

| Parameter | Value |
|---|---|
| Ionizable lipid:mRNA weight ratio | 10:1 |
| Ionizable lipid molar % | 20% to 30% |
| Phospholipid molar % | 30% to 40% |
| DMPE-PEG2k molar % | 2.5% |
| Cholesterol molar % | Rest |

TABLE 4
Full factorial formulations for lipid AMG104

| | | Ionizable | |
|---|---|---|---|
| Formulation | Helper % | Lipid % | Cholesterol % |
| 1 (Control) | 16 | 35 | 46.5 |
| 2 | 35 | 30 | 32.5 |
| 3 | 30 | 30 | 37.5 |
| 4 | 40 | 20 | 37.5 |
| 5 | 35 | 25 | 37.5 |
| 6 | 30 | 20 | 47.5 |
| 7 | 40 | 30 | 27.5 |
| 8 | 30 | 25 | 42.5 |
| 9 | 35 | 20 | 42.5 |
| 10 | 40 | 25 | 32.5 |

The results revealed that formulation 2 performed significantly better than the control formulation 1. For AMG104, formulation 2 performed ˜8.2× better than the original formulation. AMG104 using formulation 2 provided extremely potent in vitro transfection, which may be useful for applications like in vitro to hematopoietic stem cells or immune cells.

### Evaluation of the LNPs Via Intravenous Injection (IV)

After screening the lipids in vitro, the top-performing lipids were screened in vivo. Nanoparticles were administered IV in mice at a dose of 0.75 mg/kg (15 μg total mRNA). 24 hrs after dosing, mice were injected intraperitoneally with a luciferin solution. After 15 minutes, the mice were sacrificed, and their heart, lungs, kidneys, liver, and spleen collected, and the luminescence imaged using an IVIS system. For all the tested lipids, luminescence was only observed in the liver and spleen.

### Further Derivatization of AMG1041

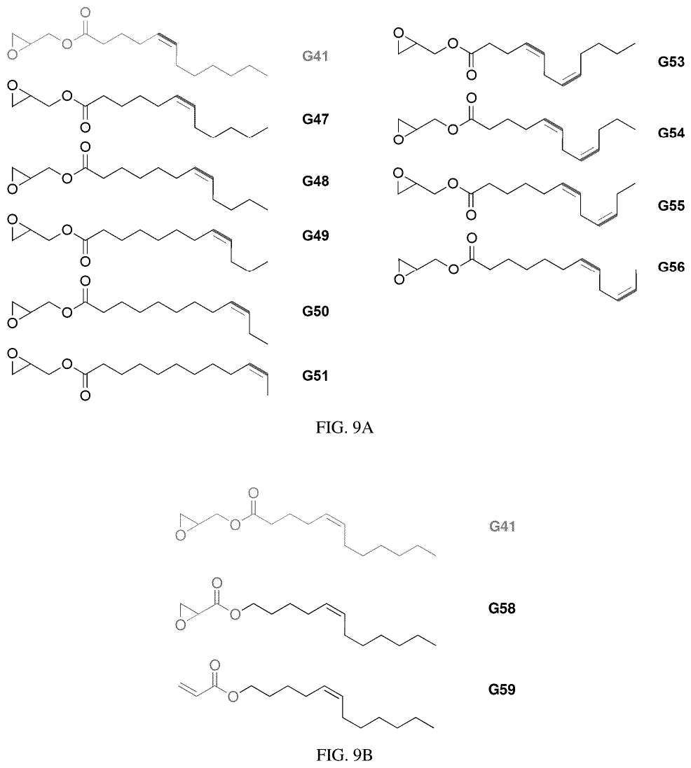

Further derivatization of the chemical structure of AMG1041 was performed by moving the Z-alkene along the G41 tail length. This process is called the “alkene walking” strategy to improve performance. Moreover, an additional Z-alkene was added, spaced by a single methylene to prevent π conjugation, and then a similar “alkene walking” strategy was applied. The structures of the relevant tails are shown in FIG. 9A.

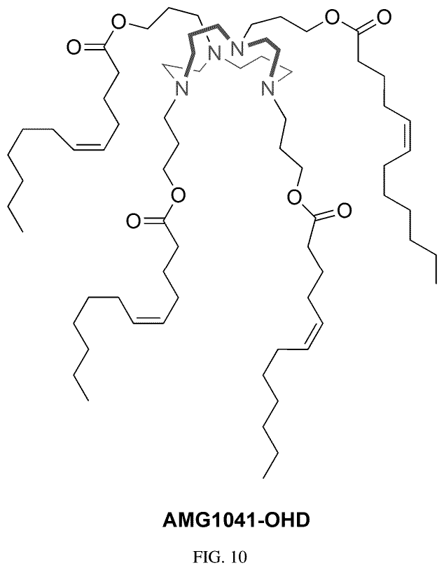

In addition, the importance of the OH group was evaluated. The importance of the orientation of the ester and OH was also evaluated (FIG. 10). The structures of the relevant tails are shown in FIG. 9B. The additional tails were also used to synthesize AMG1058, AMG1059, and AMG1041OHD. From a screening experiment using 0.1 μg FLuc mRNA dose, it was determined that the ester's specific orientation and the OH groups' location are important for G41 potency and that minor alterations can dramatically decrease potency.

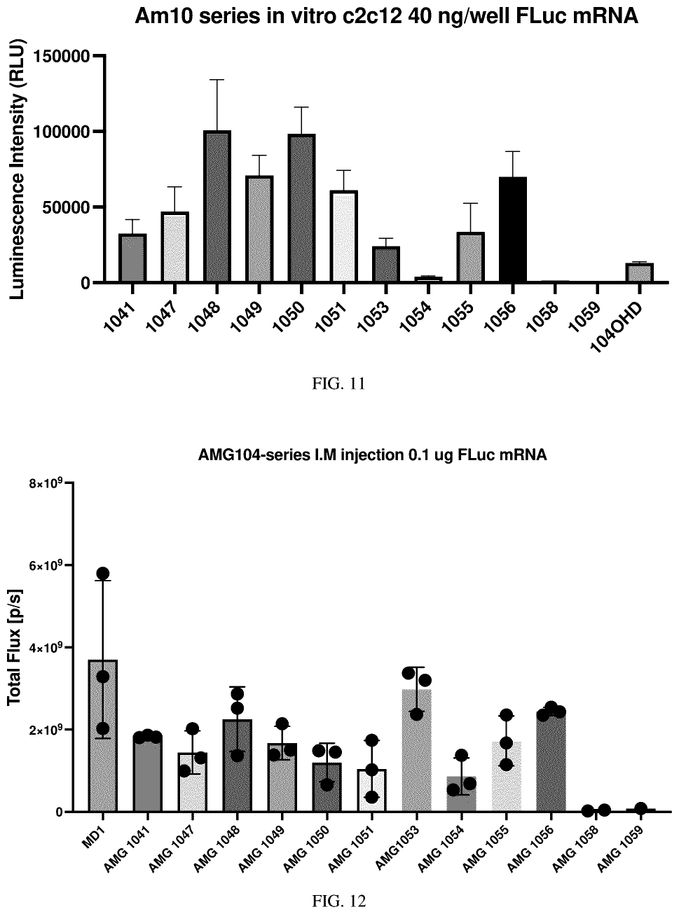

Initial in vitro testing in C2C12 myoblast cell line identified lipids that outperformed AMG1041, except for AMG1054, 1058, 1059, and 1041OHD (FIG. 11). The potency of these lipids was tested in vivo via intramuscular administration. AMG1041OHD was not tested in vivo due to difficulty in formulating these particles. The FLuc mRNA (0.1 μg) intramuscular route screening data is shown in FIG. 12 for the AMG1041 derivative lipids. AMG1053 performed nearly as well as the control sample with MD-1. AMG1048 and AMG1056 also performed similarly.

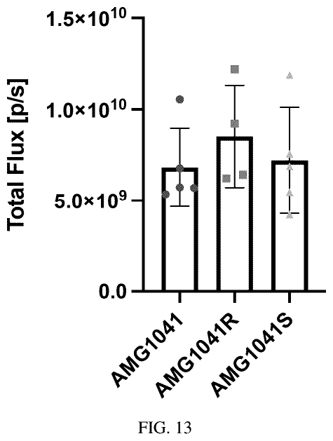

Chirally pure G41 tails did not necessarily indicate any superior activity over the diastereomeric mixture of AMG1041 based on FLuc mRNA transfection data from intramuscular injection (FIG. 13).

### Vaccine Application

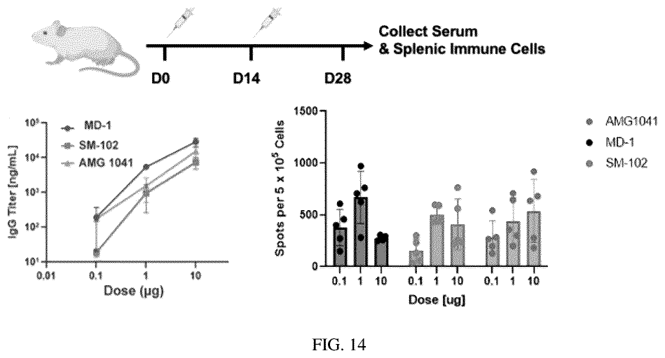

AMG1041 was tested for vaccination against ovalbumin. After intramuscular injection with varying Ova mRNA doses, antibody titers were measured at day 14, which showed similar potency compared to MD-1 and higher potency compared to SM-102. After day 28, a strong cellular response from Elispot assay was observed as compared to SM-102 and MD-1. See FIG. 14.

### Biodegradability

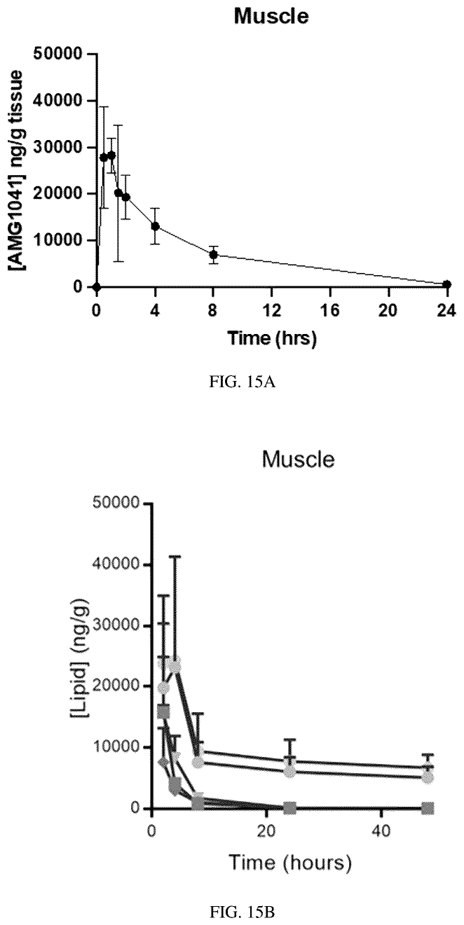

AMG1041 lipid clearance was monitored by LC-MS after intramuscular injection to BALB/C mice at various time points. AMG1041 was cleared within 24 hours from the injection site in the muscle (FIG. 15A). As a comparison, SM-102 was detectable even after 40 hours (FIG. 15B).⁵ https://www.ncbi.nlm.nih.gov/pmc/articles/PMC6383180/).

### REFERENCES

1 Love, K. T. et al. Lipid-like materials for low-dose, in vivo gene silencing. Proc. Natl. Acad. Sci. U.S.A. 107, 1864-1869, (2010).
2 Semple, S. C. et al. Rational design of cationic lipids for siRNA delivery. Nat. Biotechnol. 28, 172-176, (2010).
3 Kauffman, K. J. et al. Optimization of Lipid Nanoparticle Formulations for mRNA Delivery in Vivo with Fractional Factorial and Definitive Screening Designs. Nano Lett. 15, 7300-7306, (2015).
4 Billingsley, M. M. et al. Ionizable Lipid Nanoparticle-Mediated mRNA Delivery for Human CAR T Cell Engineering. Nano Lett. 20, 1578-1589, (2020).
5 Hassett, K. K. et al. Optimization of Lipid Nanoparticles for Intramuscular Administration of mRNA Vaccines. Mol. Ther. Nucleic Acids. 2019, 15, 1-11.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 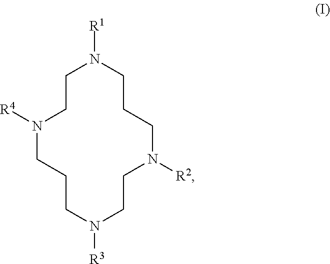
or a pharmaceutically acceptable salt thereof,
wherein:
each of R¹, R², R³, and R⁴ is independently hydrogen, a nitrogen protecting group, or 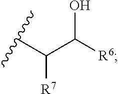
one of R⁶ and R⁷ is hydrogen, and the other is 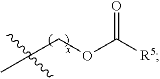
each instance of R⁵ is independently optionally substituted C4-C25 saturated or unsaturated aliphatic or optionally substituted C4-C25 saturated or unsaturated heteroaliphatic; and
each x independently is an integer from 1 to 10, inclusive;
provided that at least one of R¹, R², R³, and R⁴ is

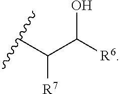

2-8. (canceled)

9. The compound of claim 1, or a pharmaceutically acceptable salt thereof, wherein each R⁵ is independently optionally substituted C₄-C₂₅ alkyl or optionally substituted C₄-C₂₅ alkenyl.

10-11. (canceled)

12. The compound of claim 1, or a pharmaceutically acceptable salt thereof, wherein each R⁵ is independently optionally substituted C₄-C₂₅ heteroalkyl or optionally substituted C₄-C₂₅ heteroalkenyl.

13. (canceled)

14. The compound of claim 1, or a pharmaceutically acceptable salt thereof, wherein each R⁵ is independently selected from the group consisting of:

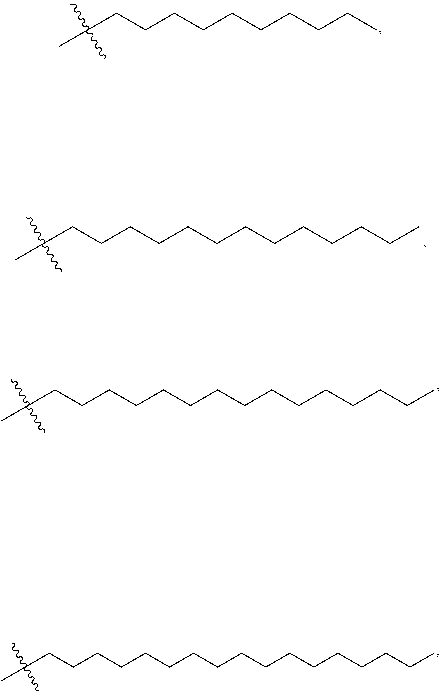 [SMILES: *CCCCCCCCCC ; *CCCCCCCCCCCCC ; *CCCCCCCCCCCCCCC ; *CCCCCCCCCCCCCCCCC]

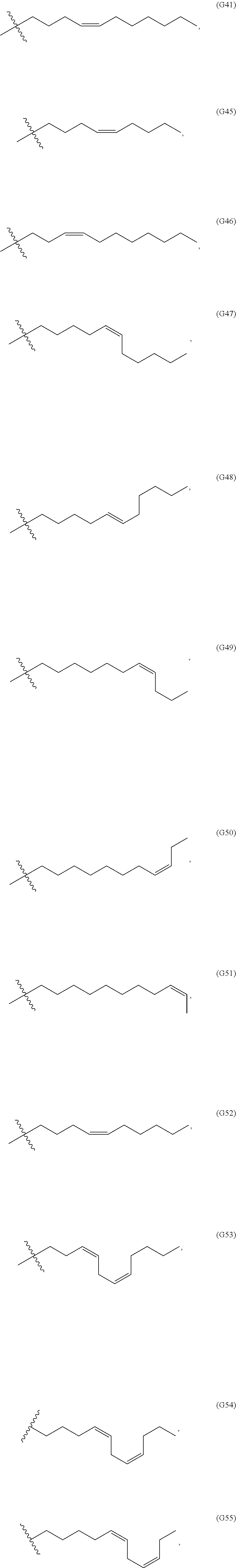

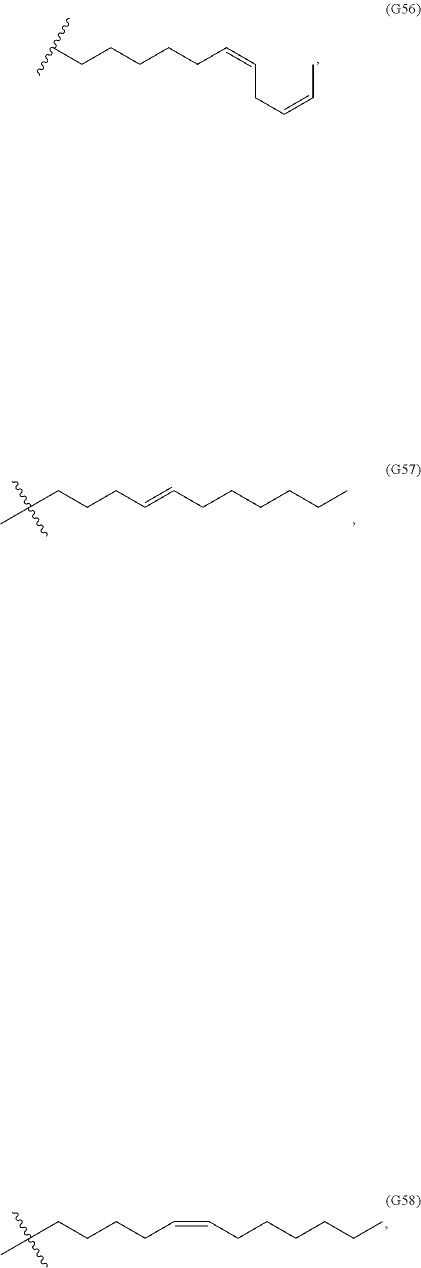 [SMILES: *CCCCC/C=C\C/C=C\C ; *CCC/C=C/CCCCCC ; *CCCC/C=C\CCCCCC]

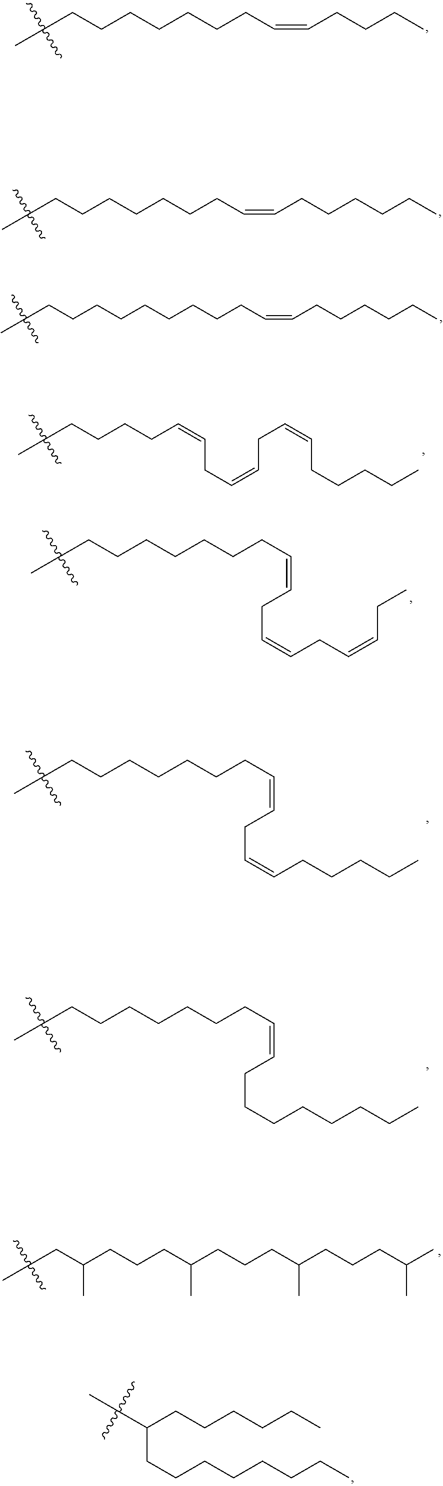

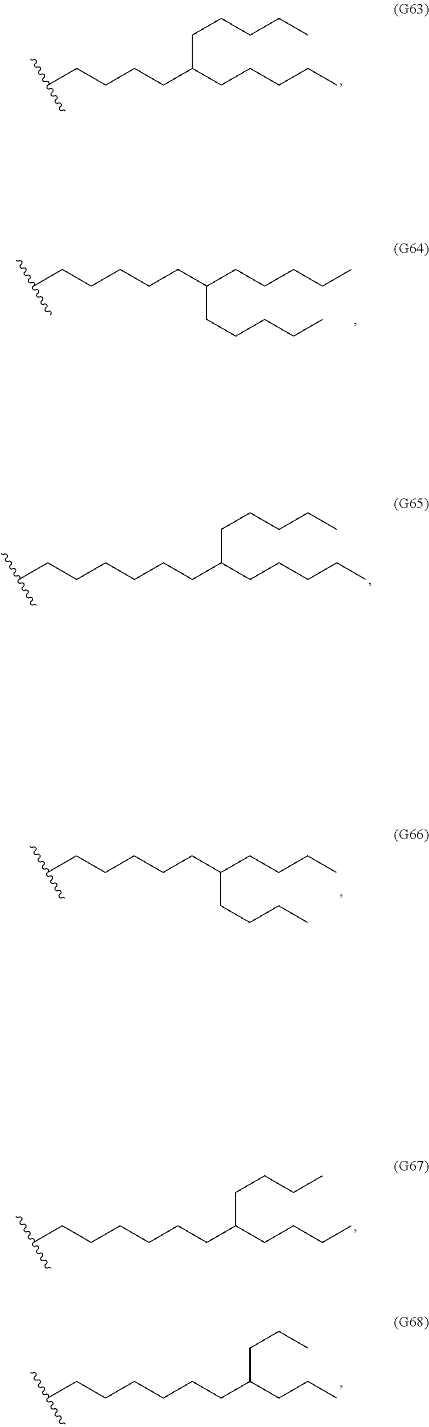 [SMILES: *CCCCC(CCCCC)CCCCC ; *CCCCCC(CCCCC)CCCCC ; *CCCCCCC(CCCCC)CCCCC ; *CCCCCC(CCCC)CCCC ; *CCCCCCC(CCCC)CCCC ; *CCCCCCC(CCC)CCC]

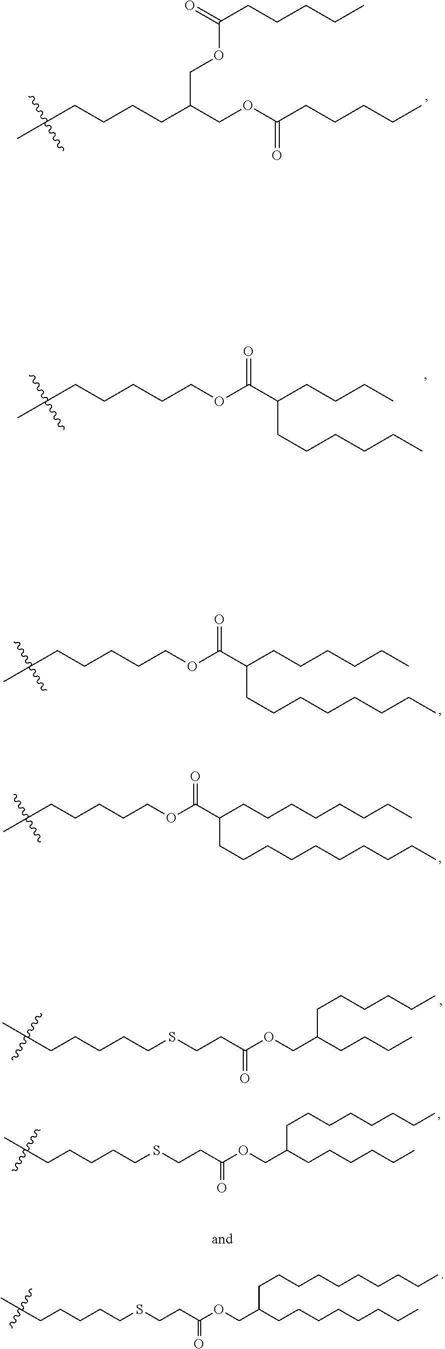

15-16. (canceled)

17. The compound of claim 1, selected from the group consisting of: 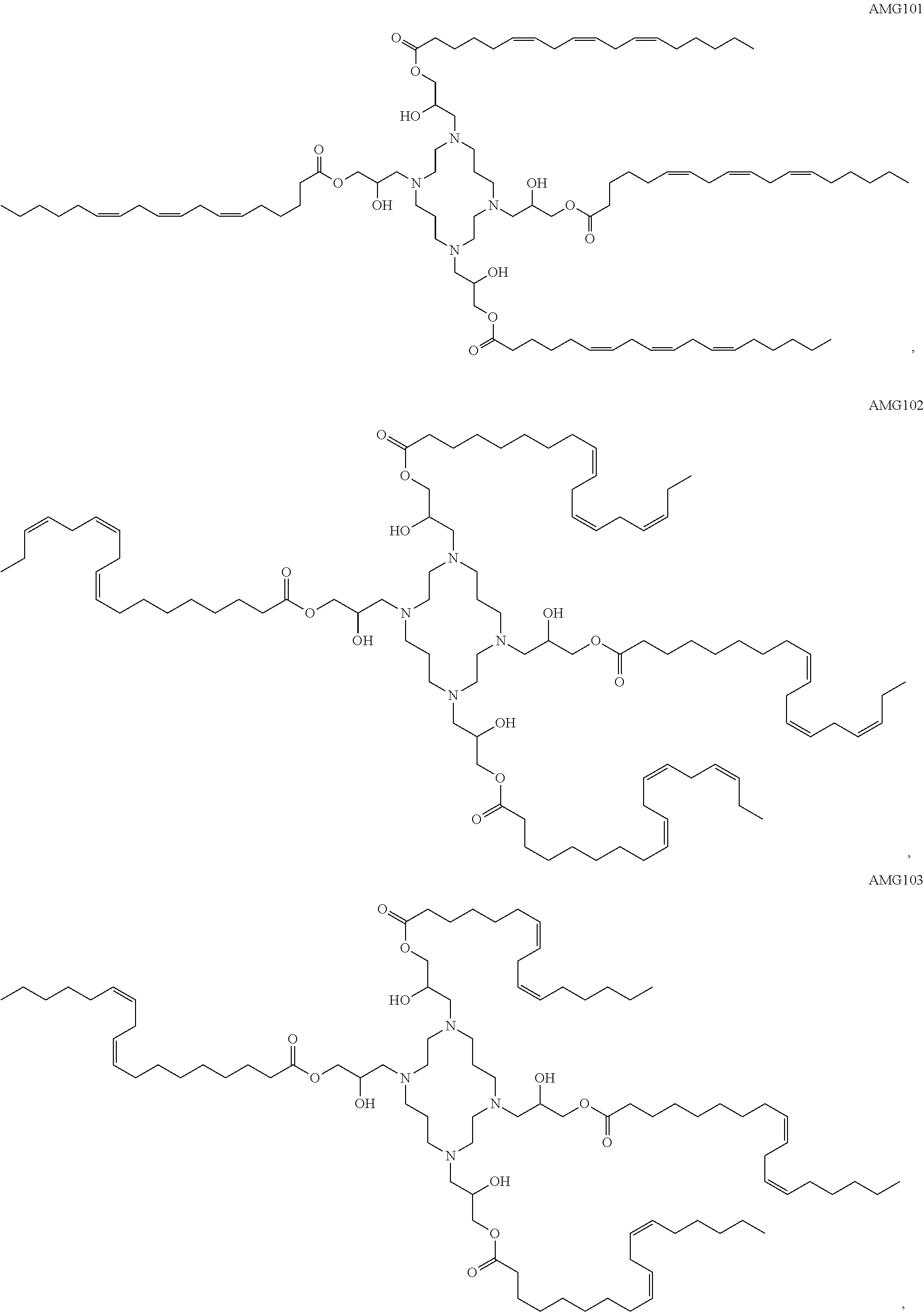 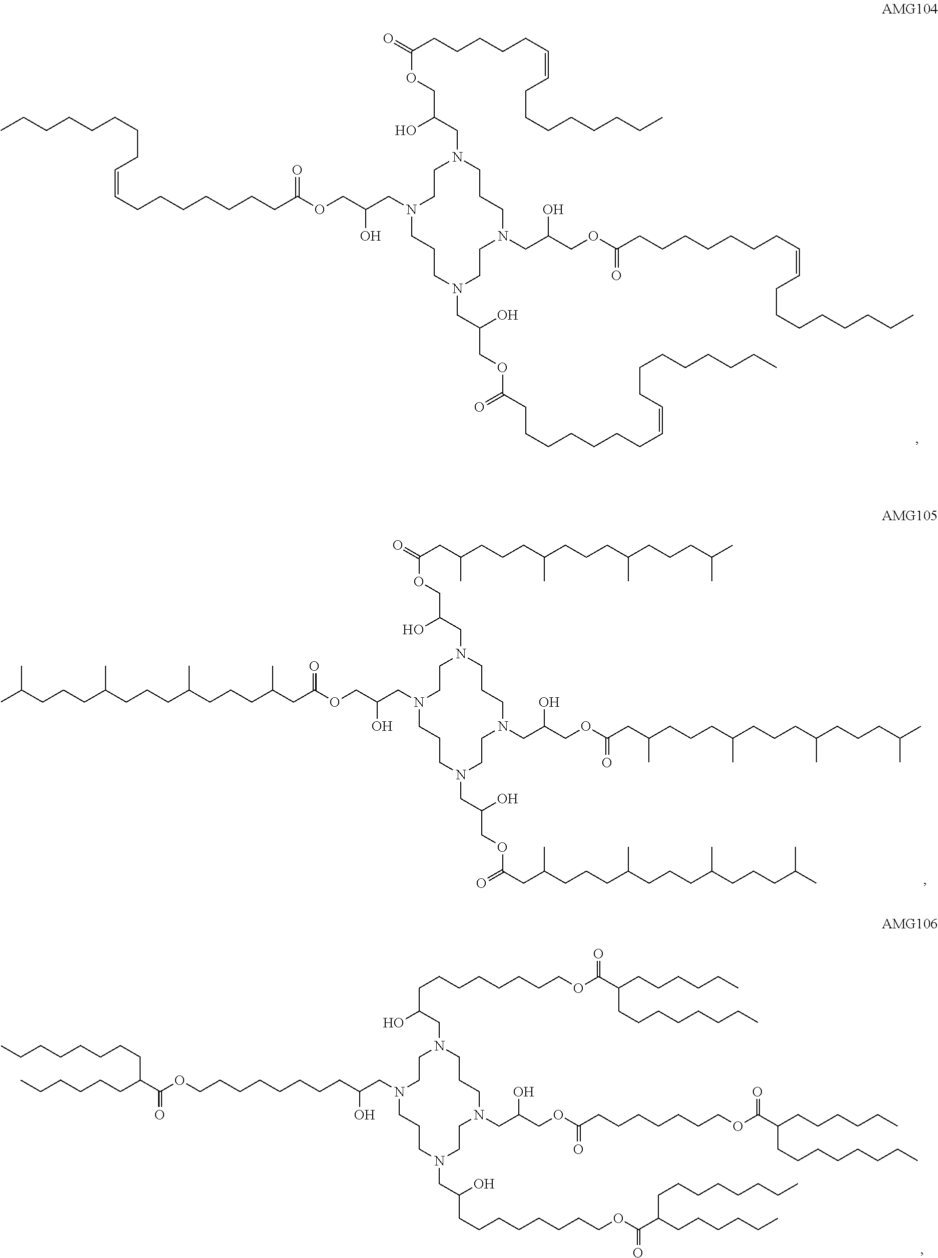 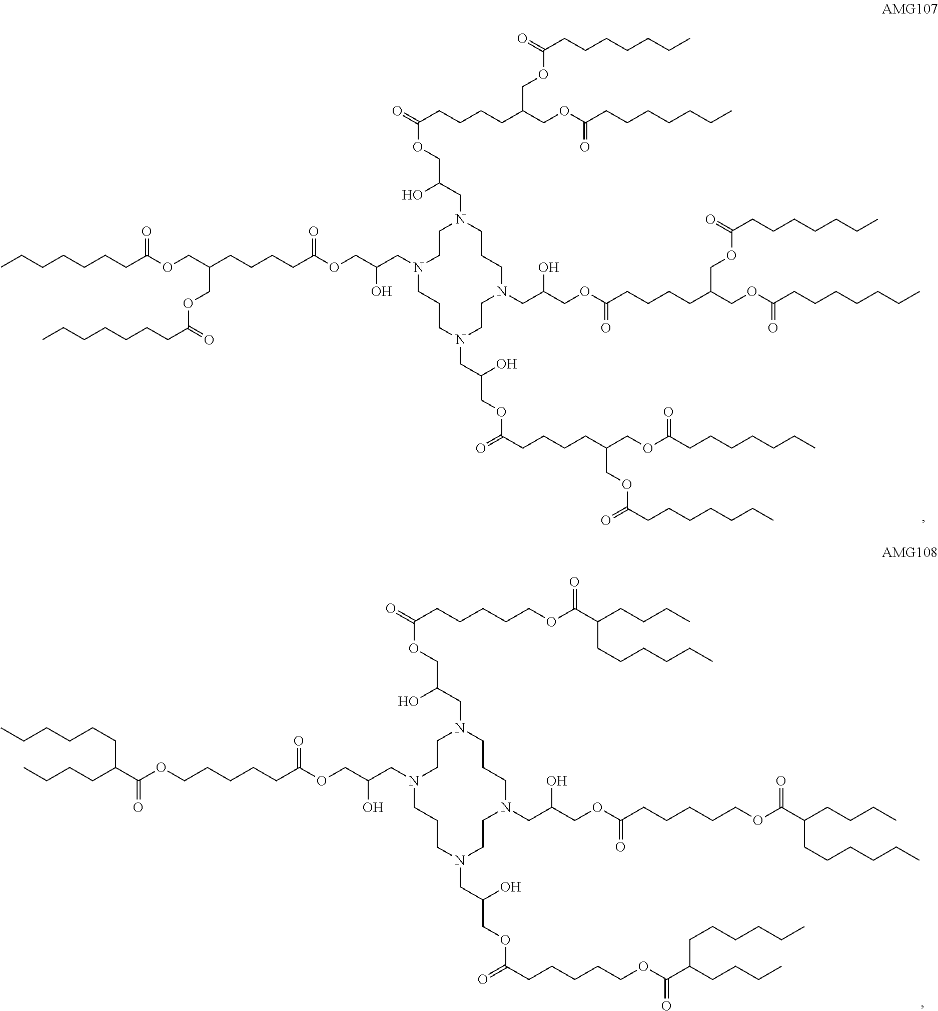 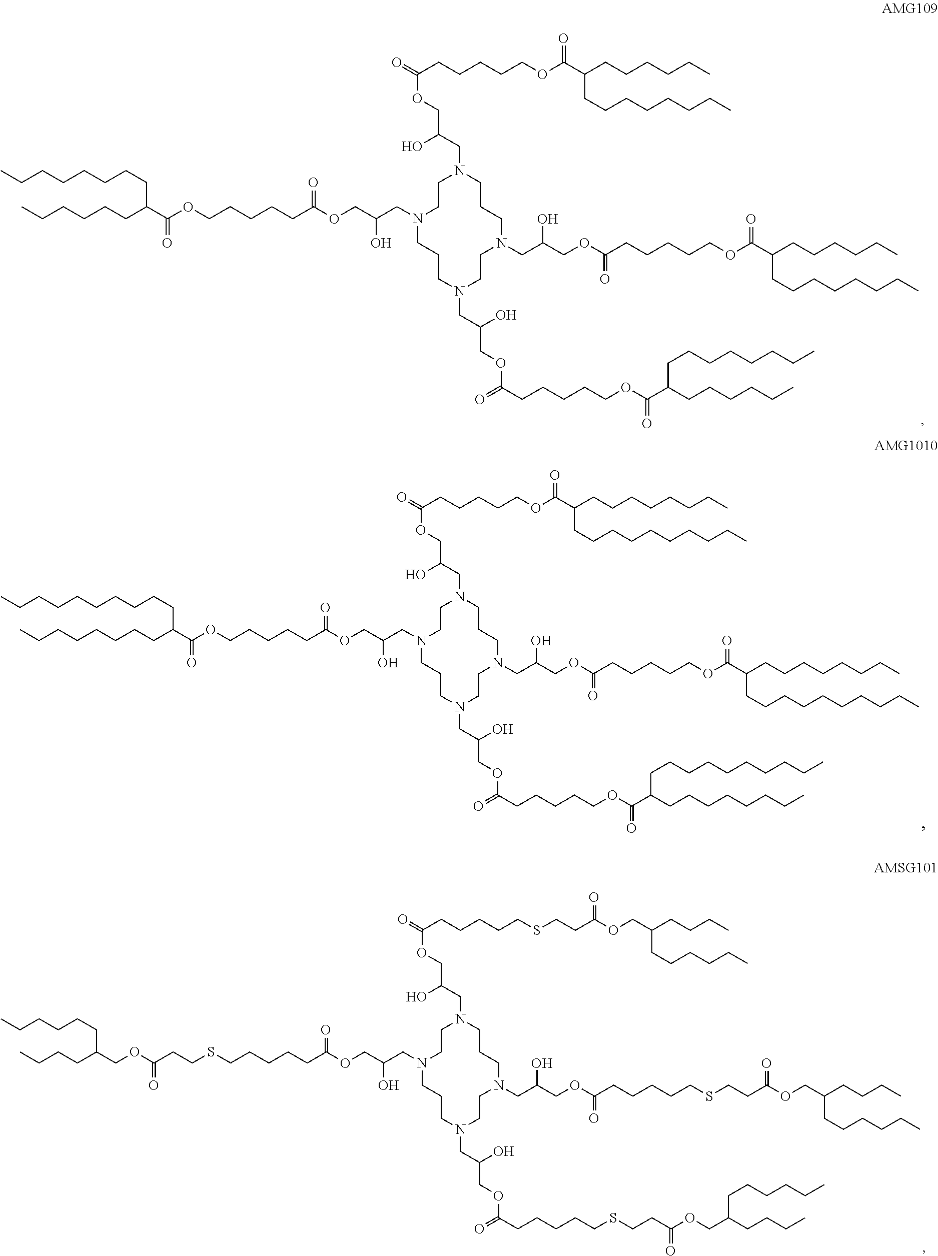 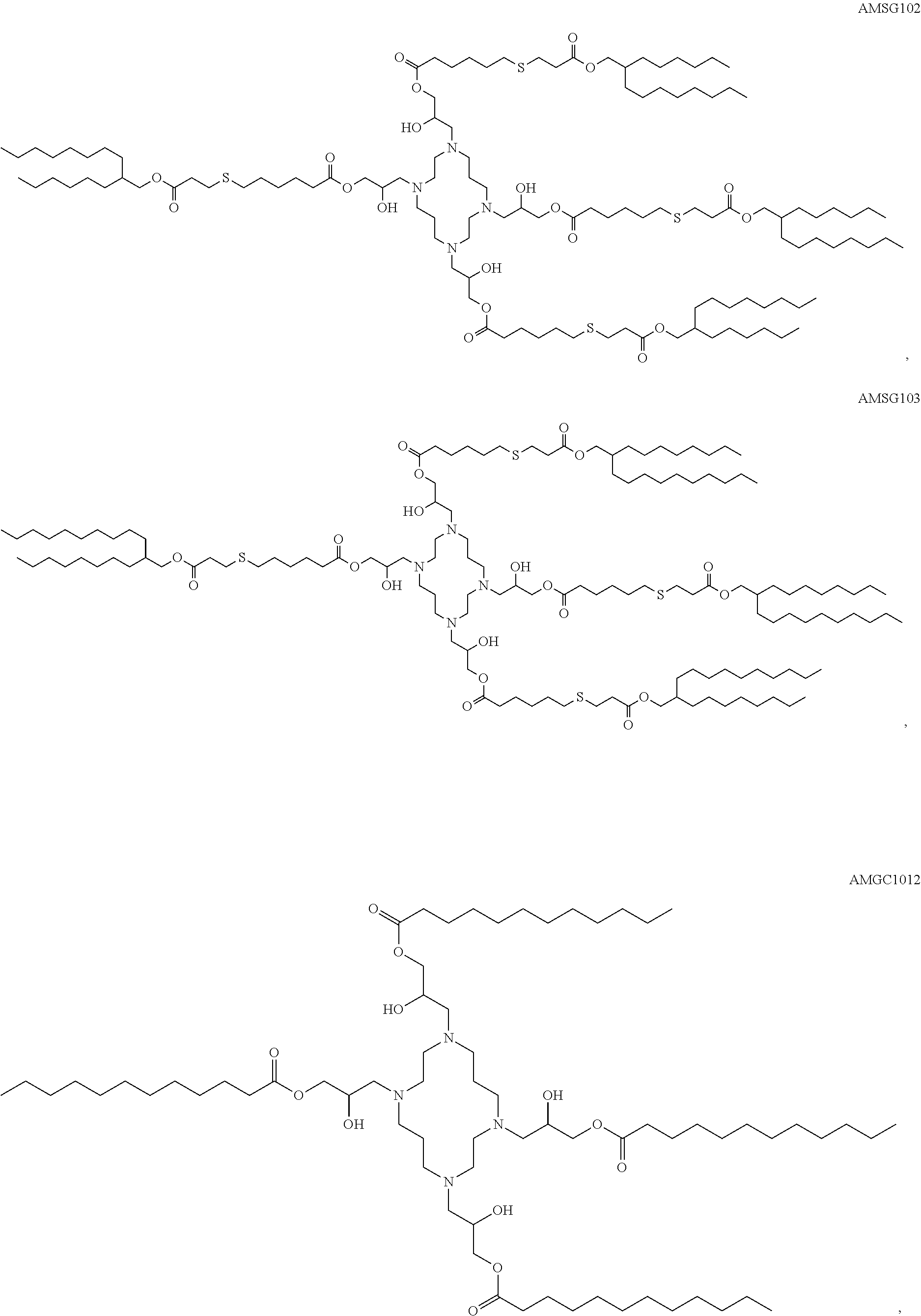 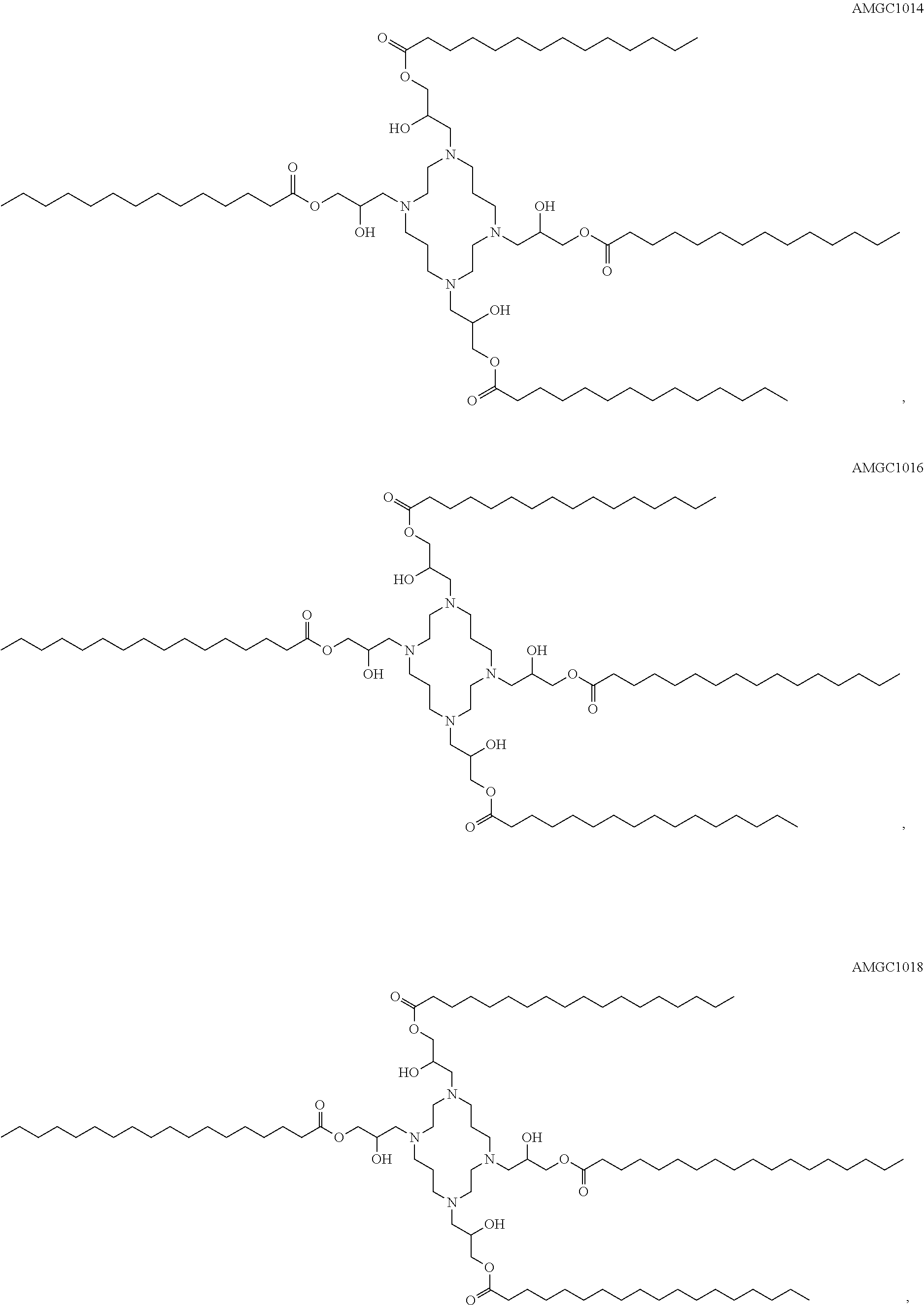 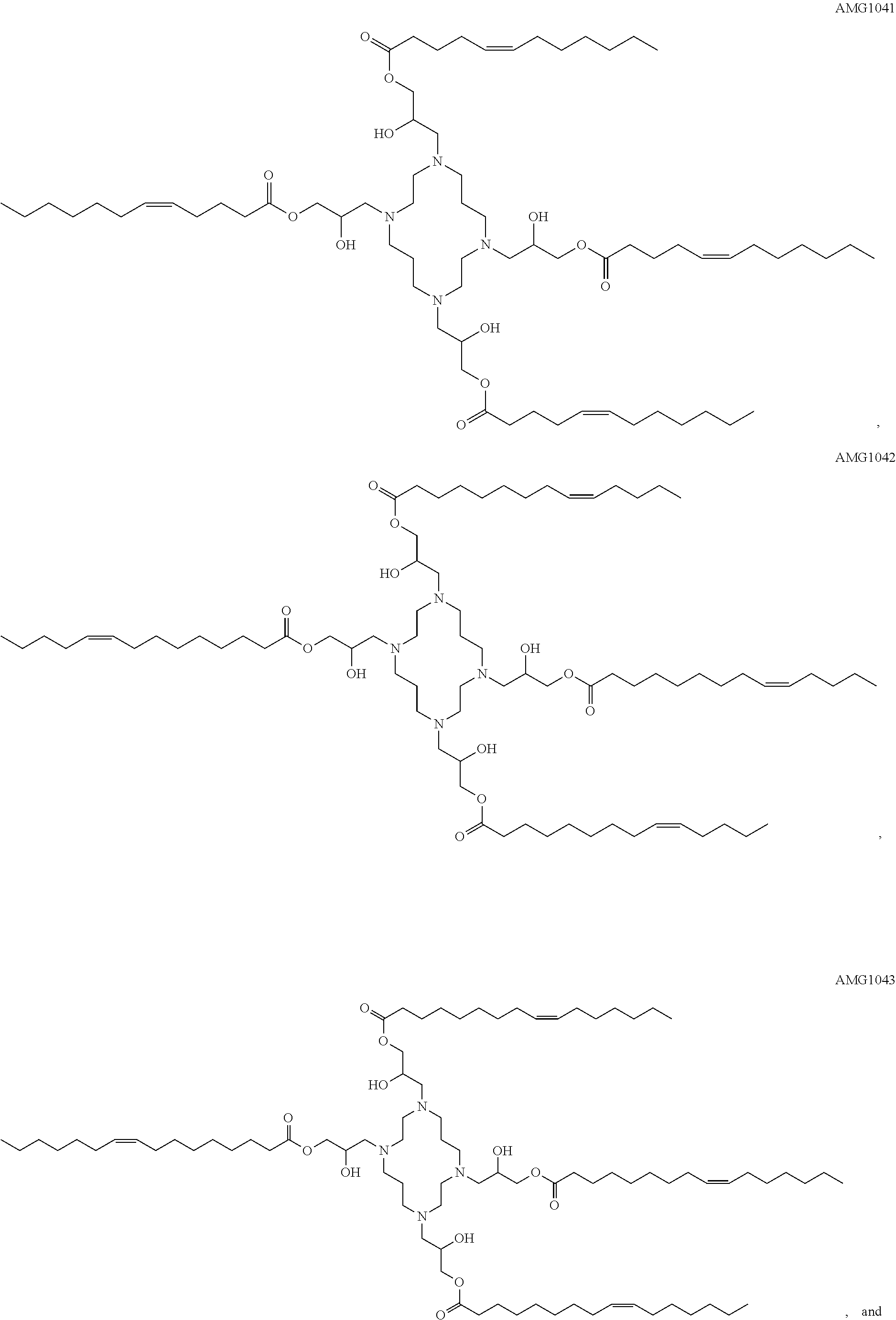 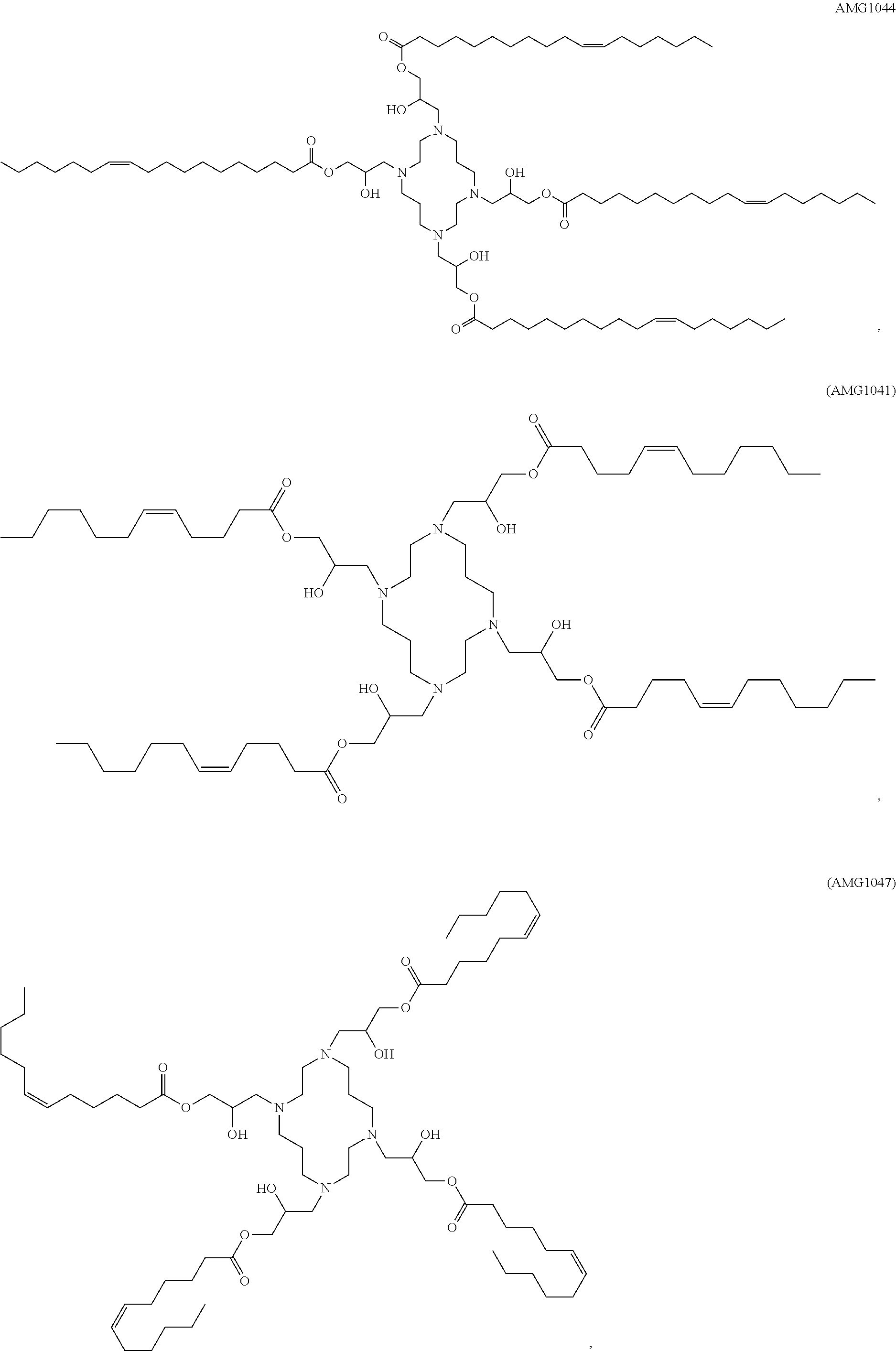 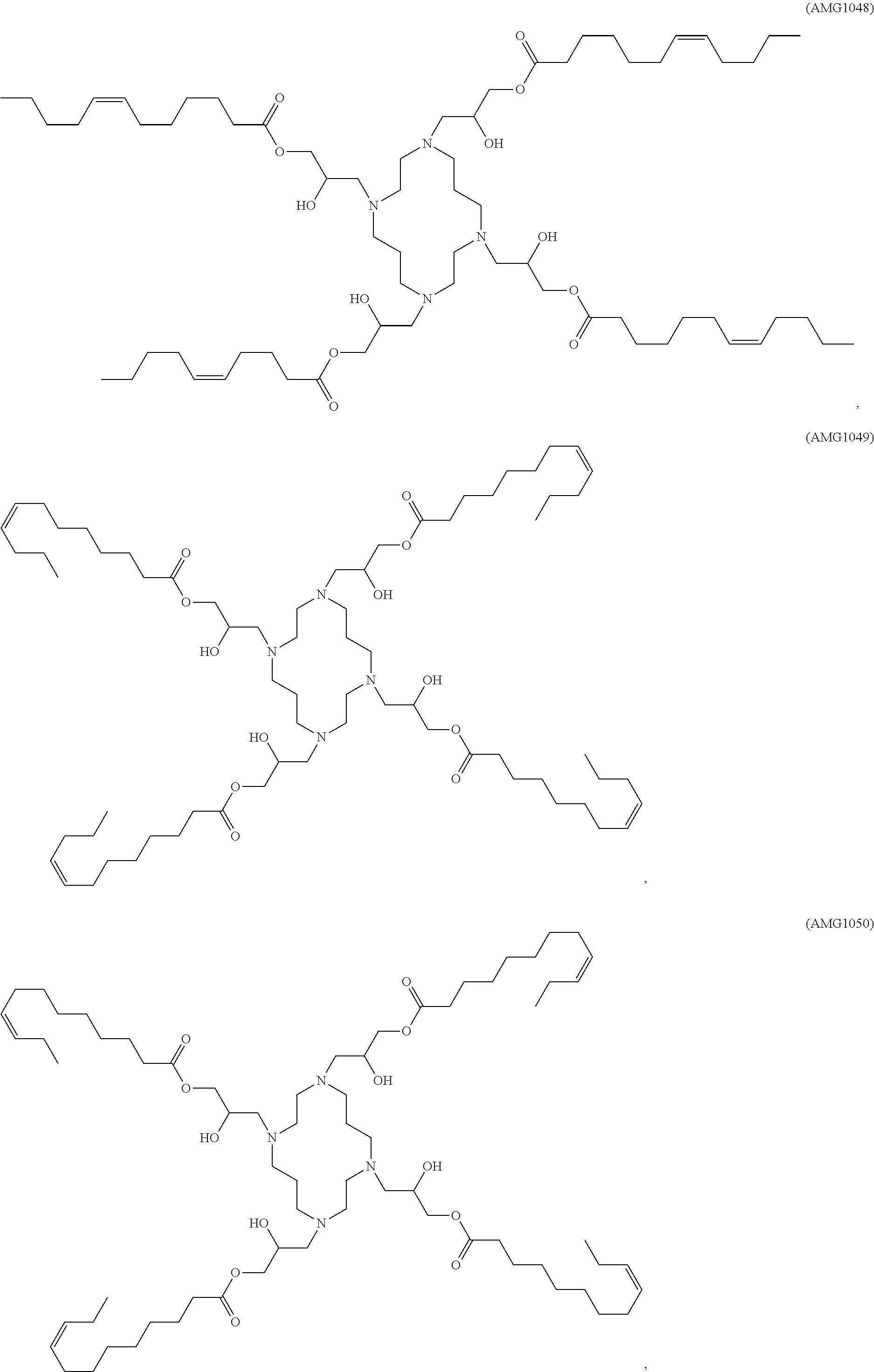 [SMILES: CCCC/C=C\CCCCCC(=O)OCC(O)CN1CCCN(CC(O)COC(=O)CCCCC/C=C\CCCC)CCN(CC(O)COC(=O)CCCCC/C=C\CCCC)CCCN(CC(O)COC(=O)CCC/C=C\CCCC)CC1 ; CCC/C=C\CCCCCCC(=O)OCC(O)CN1CCCN(CC(O)COC(=O)CCCCCC/C=C\CCC)CCN(CC(O)COC(=O)CCCCCC/C=C\CCC)CCCN(CC(O)COC(=O)CCCCCC/C=C\CCC)CC1 ; CC/C=C\CCCCCCCC(=O)OCC(O)CN1CCCN(CC(O)COC(=O)CCCCCCC/C=C\CC)CCN(CC(O)COC(=O)CCCCCCC/C=C\CC)CCCN(CC(O)COC(=O)CCCCCCC/C=C\CC)CC1] 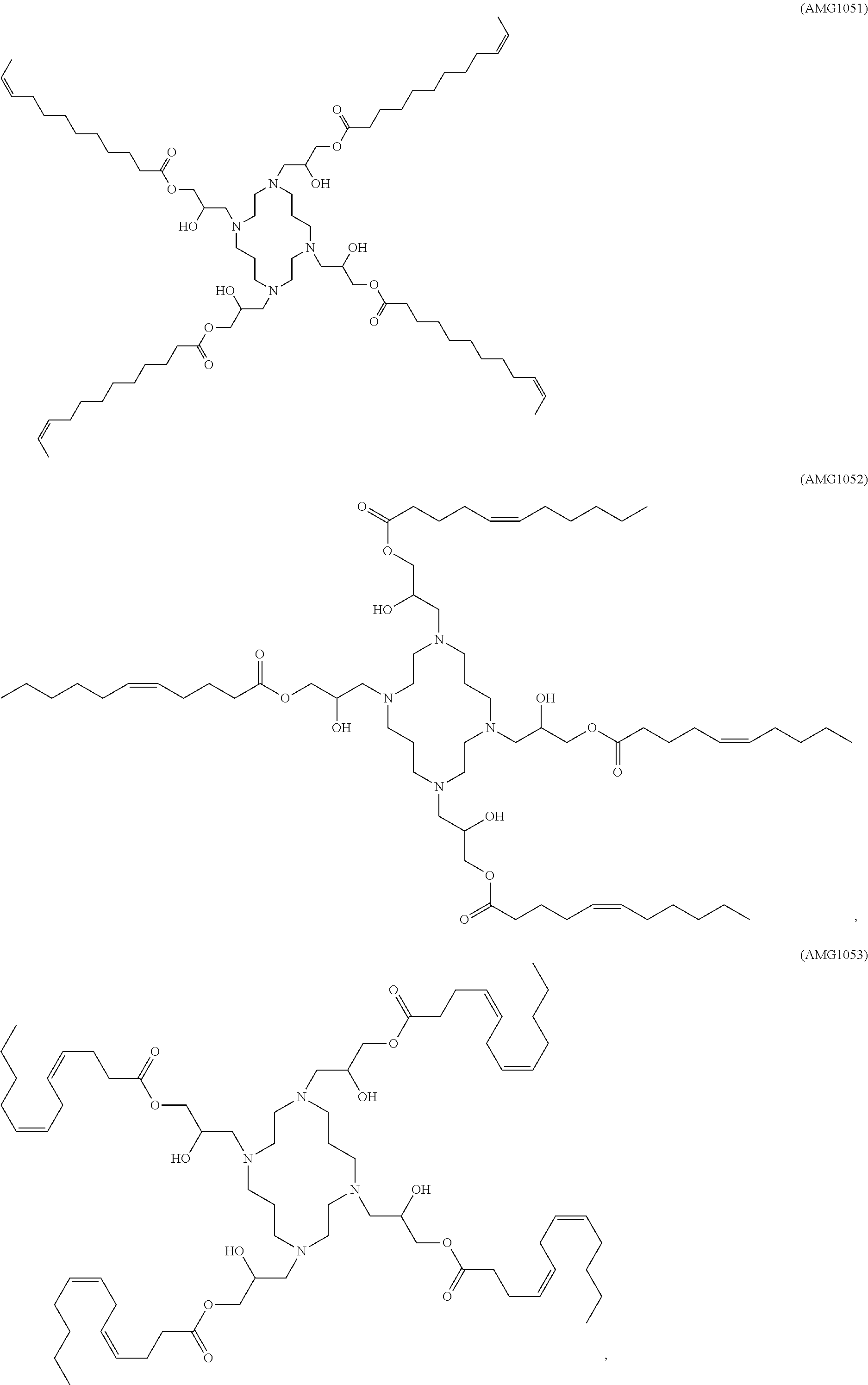 [SMILES: C/C=C\CCCCCCCCC(=O)OCC(O)CN1CCCN(CC(O)COC(=O)CCCCCCCC/C=C\C)CCN(CC(O)COC(=O)CCCCCCCC/C=C\C)CCCN(CC(O)COC(=O)CCCCCCCC/C=C\C)CC1 ; CCCC/C=C\CCCC(=O)OCC(O)CN1CCCN(CC(O)COC(=O)CCC/C=C\CCCCC)CCN(CC(O)COC(=O)CCC/C=C\CCCCC)CCCN(CC(O)COC(=O)CCC/C=C\CCCCC)CC1 ; CCCC/C=C\C/C=C\CCC(=O)OCC(O)CN1CCCN(CC(O)COC(=O)CC/C=C\C/C=C\CCCC)CCN(CC(O)COC(=O)CC/C=C\C/C=C\CCCC)CCCN(CC(O)COC(=O)CC/C=C\C/C=C\CCCC)CC1] 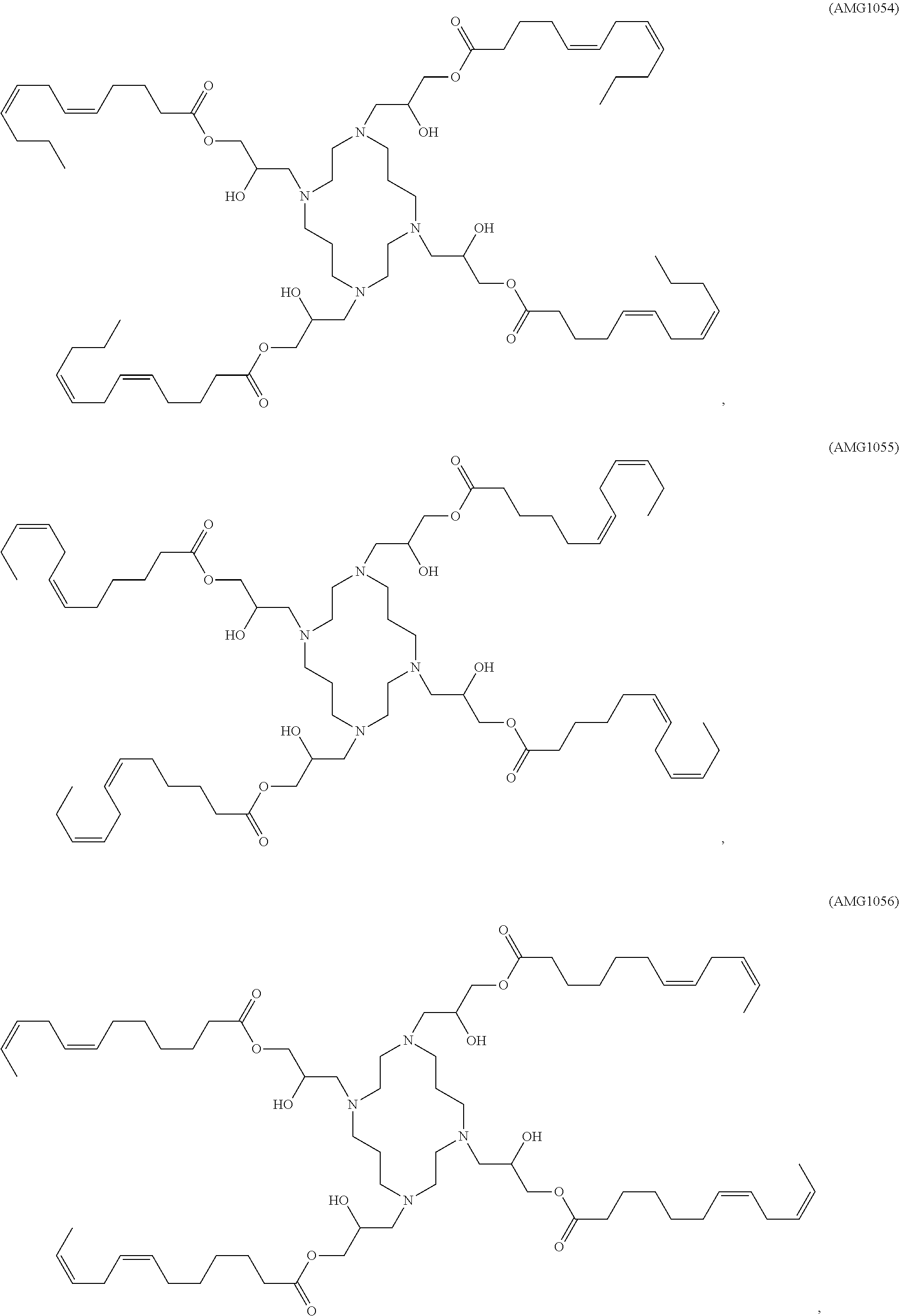 [SMILES: CCC/C=C\C/C=C\CCCC(=O)OCC(O)CN1CCCN(CC(O)COC(=O)CCC/C=C\C/C=C\CCC)CCN(CC(O)COC(=O)CCC/C=C\C/C=C\CCC)CCCN(CC(O)COC(=O)CCC/C=C\C/C=C\CCC)CC1 ; CC/C=C\C/C=C\CCCCC(=O)OCC(O)CN1CCCN(CC(O)COC(=O)CCCC/C=C\C/C=C\CC)CCN(CC(O)COC(=O)CCCC/C=C\C/C=C\CC)CCCN(CC(O)COC(=O)CCCC/C=C\C/C=C\CC)CC1 ; C/C=C\C/C=C\CCCCCC(=O)OCC(O)CN1CCCN(CC(O)COC(=O)CCCCC/C=C\C/C=C\C)CCN(CC(O)COC(=O)CCCCC/C=C\C/C=C\C)CCCN(CC(O)COC(=O)CCCCC/C=C\C/C=C\C)CC1] 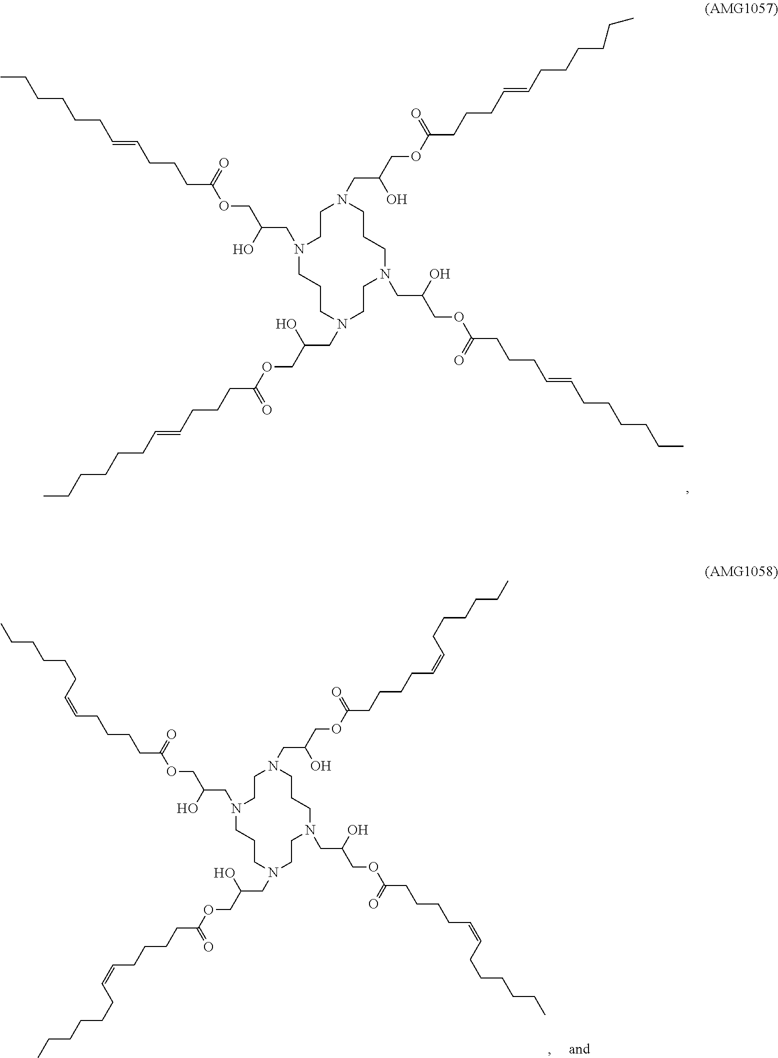 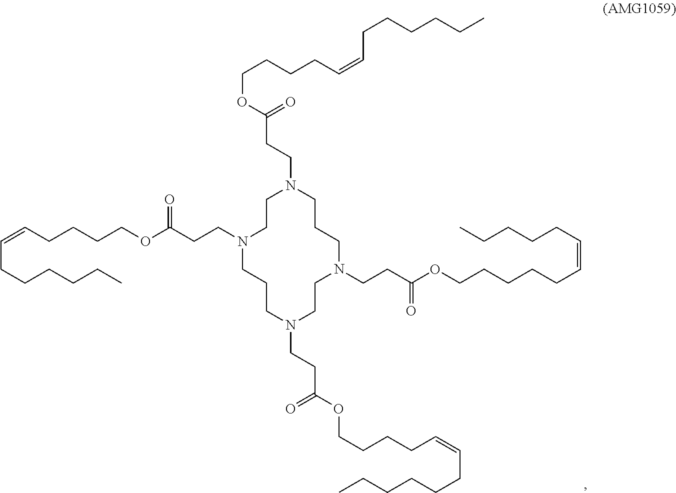 [SMILES: CCCCC/C=C\CCCCCOC(=O)CCN1CCCN(CCC(=O)OCCCC/C=C\CCCCCC)CCN(CCC(=O)OCCCC/C=C\CCCCCC)CCCN(CCC(=O)OCCCC/C=C\CCCCCC)CC1]
or a pharmaceutically acceptable salt thereof.

18. The compound of claim 1, selected from the group consisting of: 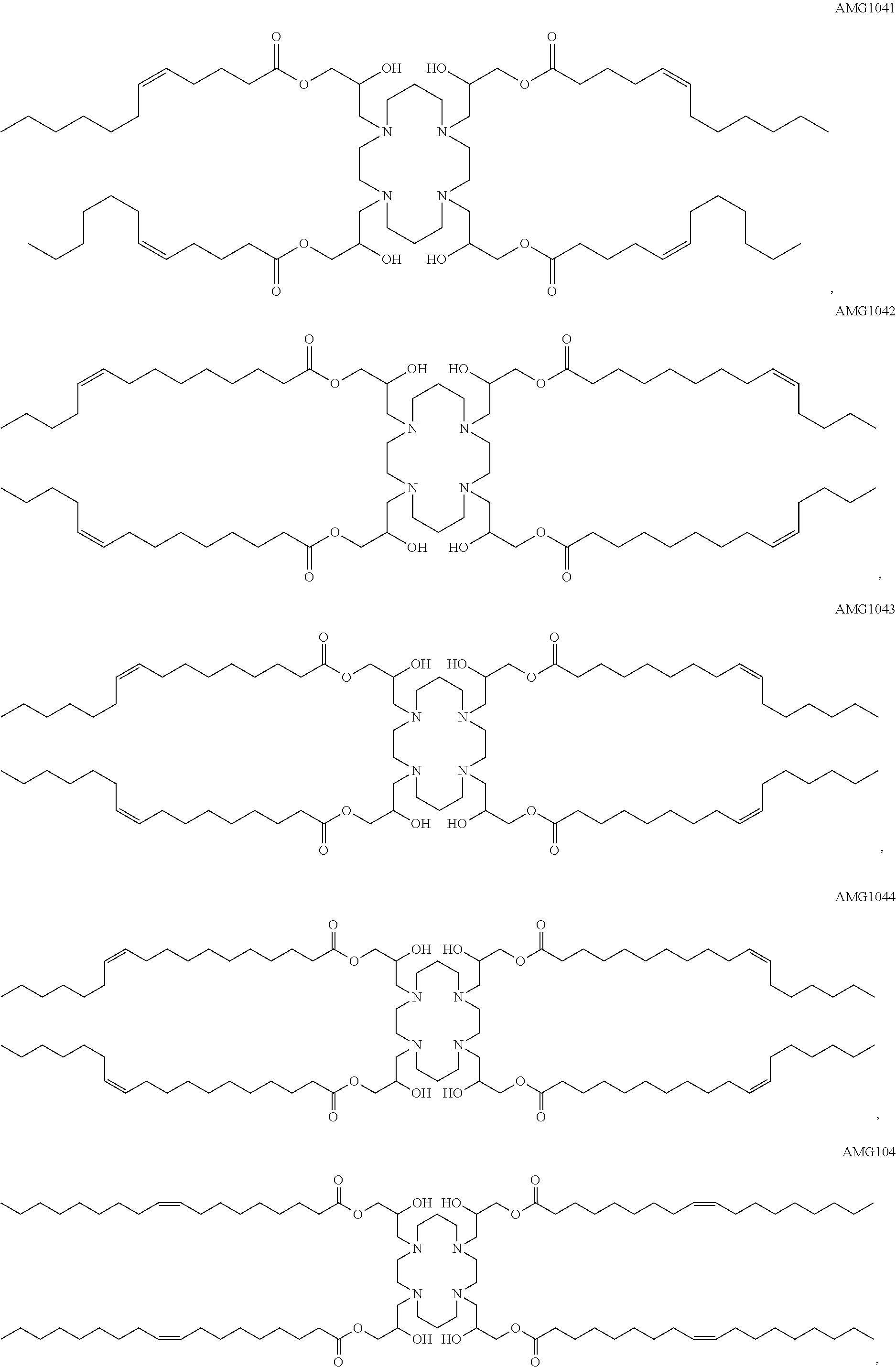 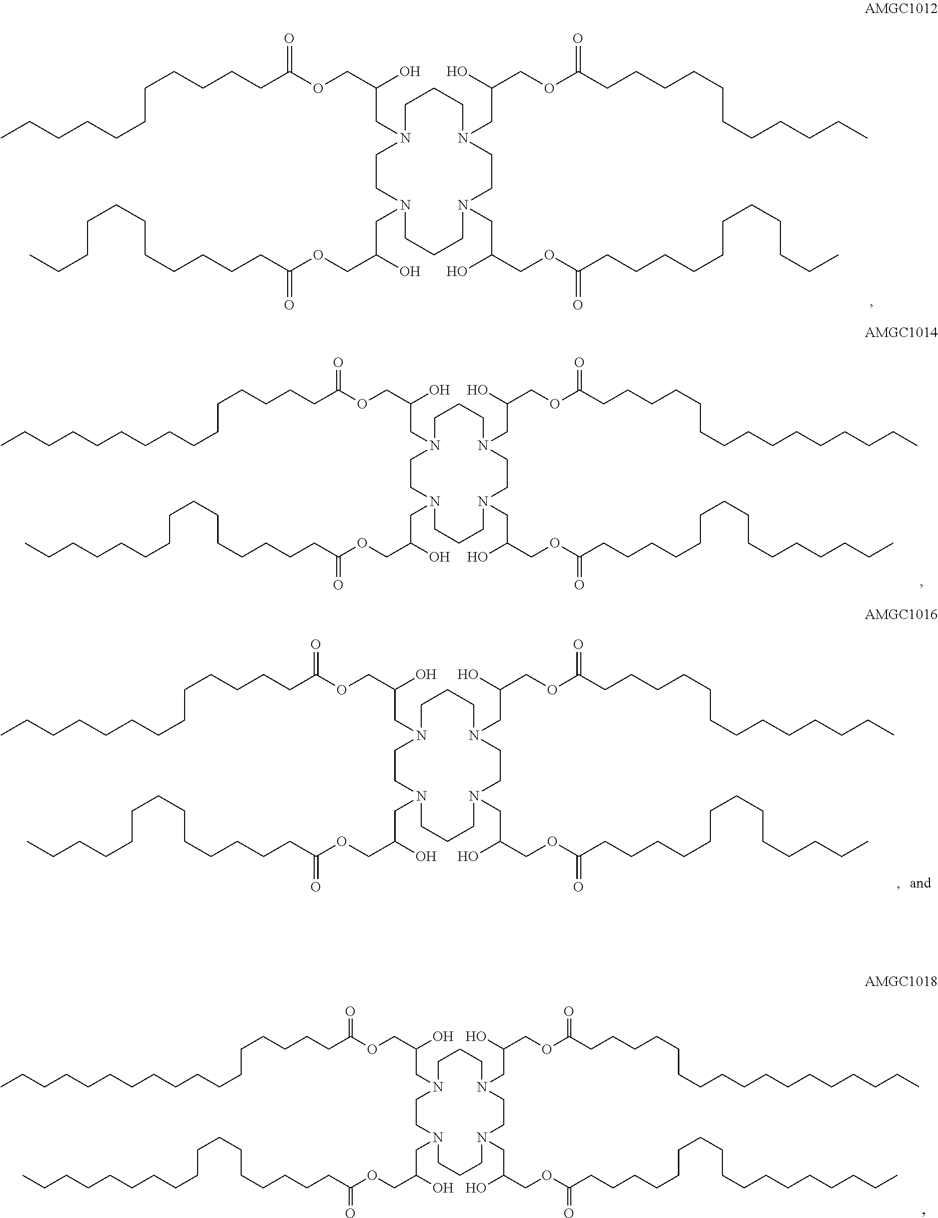
or a pharmaceutically acceptable salt thereof.

19-22. (canceled)

23. A composition comprising a compound of claim 1, or a pharmaceutically acceptable salt thereof, and a pharmaceutically acceptable excipient.

24. The composition of claim 23, wherein the composition further comprises an agent.

25. The composition of claim 24, wherein the agent is an organic molecule, inorganic molecule, polynucleotide, protein, peptide, polynucleotide, targeting agent, an isotopically labeled chemical compound, vaccine, an immunological agent, or an agent useful in bioprocessing.

26. The composition of claim 24, wherein the agent is a polynucleotide.

27. The composition of claim 26, wherein the polynucleotide is an RNA.

28. (canceled)

29. The composition of claim 27, wherein the RNA is messenger RNA (mRNA).

30. The composition of claim 27, wherein the RNA is precursor messenger RNA.

31. The composition of claim 27, wherein the RNA is double-stranded RNA, short hairpin RNA, microRNA, guide RNA, transfer RNA, antisense RNA, long non-coding RNA, signal recognition particle RNA, small cytoplasmic RNA, small nuclear RNA, ribosomal RNA, Piwi-interacting RNA, small nucleolar RNA, or spliced leader RNA.

32-34. (canceled)

35. The composition of claim 26, wherein the polynucleotide is a DNA.

36. (canceled)

37. The composition of claim 23, wherein the composition further comprises one or more of a PEG-lipid, sterol, or phospholipid.

38-45. (canceled)

46. A method of delivering an agent to a subject or a cell, comprising administering to the subject or contacting the cell with a composition comprising an agent and a compound of claim 1, or a pharmaceutically acceptable salt thereof.

47-60. (canceled)

61. A method of treating and/or preventing a disease, disorder, or condition in a subject, comprising administering to the subject a composition comprising an agent and a compound of claim 1, or a pharmaceutically acceptable salt thereof.

62-63. (canceled)

64. A method of preparing a compound of Formula (II): 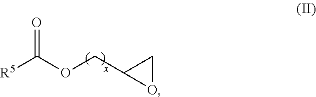
the method comprising reacting a compound of Formula (III): 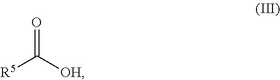
or a salt thereof, with a carbodiimide coupling agent, or a salt thereof, and a compound of Formula (IV): 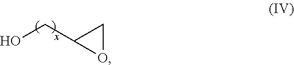
or a salt thereof, wherein:
x is an integer from 1 to 10, inclusive; and
R⁵ is optionally substituted C₄-C₂₅ saturated or unsaturated aliphatic or optionally substituted C₄-C₂₅ saturated or unsaturated heteroaliphatic.

65-74. (canceled)

75. A method of preparing a compound of Formula (I): 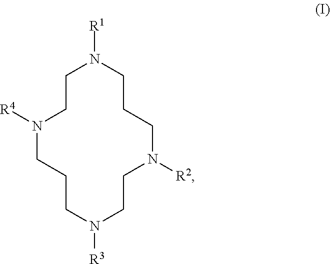
or a salt thereof, the method comprising reacting a compound of Formula (II): 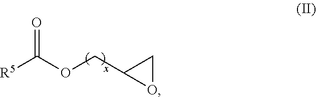
with a compound of Formula (V): 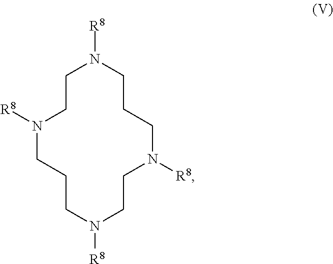
or a salt thereof, wherein:
each of R¹, R², R³, and R⁴ is independently hydrogen, a nitrogen protecting group, or 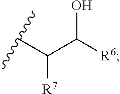
one of R⁶ and R⁷ is hydrogen, and the other is 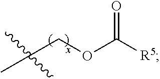
each instance of R⁵ is independently optionally substituted C₄-C₂₅ saturated or unsaturated aliphatic or optionally substituted C₄-C₂₅ saturated or unsaturated heteroaliphatic; and
each x independently is an integer from 1 to 10, inclusive;
provided that at least one of R¹, R², R³, and R⁴ is 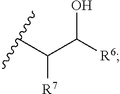
and
each instance of R⁸ is independently hydrogen or a nitrogen protecting group.

76-81. (canceled)
